(19) United States
(12) Patent Application Publication
(10) Pub. No.: US 2024/0307521 A1
(43) Pub. Date: Sep. 19, 2024

(54) RNA VACCINES AGAINST INFECTIOUS DISEASES

(71) Applicant: HDT Bio Corp., Seattle, WA (US)

(72) Inventors: Steven Gregory Reed, Bellevue, WA (US); Darrick Albert Carter, Seattle, WA (US); Amit Praful Khandhar, Issaquah, WA (US); Jacob Freeman Archer, Seattle, WA (US); Lars Peter Aksel Berglund, Seattle, WA (US); Jesse Erasmus, Port Orchard, WA (US); Bryan Berube, Issaquah, WA (US); Malcolm S. Duthie, Sammamish, WA (US)

(21) Appl. No.: 18/610,681

(22) Filed: Mar. 20, 2024

Related U.S. Application Data
(63) Continuation of application No. PCT/US2022/013516, filed on Jan. 24, 2022.
(60) Provisional application No. 63/247,175, filed on Sep. 22, 2021.
(60) Provisional application No. 63/297,498, filed on Jan. 7, 2022.

(51) Int. Cl. A61K 39/145; A61K 9/127; A61K 9/51; A61K 39/00; A61K 47/02; A61K 47/10

(52) U.S. Cl. CPC A61K 39/145; A61K 9/127; A61K 9/5123; A61K 47/02; A61K 47/10; A61K 2039/53; A61K 2039/55505; A61K 2039/55555; A61K 2039/55566; C12N 2760/16134; C12N 2760/16234

(57) ABSTRACT

The disclosure provides compositions, methods of treatment, and methods of making and using compositions to deliver a nucleic acid to a subject. Methods of using these compositions as a vaccine for treatment of an infectious disease are also provided.

### CROSS-REFERENCE

This application is a continuation of International Application No. PCT/US2022/013516, filed Jan. 24, 2022, which claims the benefit of priority to U.S. Provisional Patent Application No. 63/247,175, filed Sep. 22, 2021, and U.S. Provisional Patent Application No. 63/297,498, filed on Jan. 7, 2022, the contents of each of which are incorporated herein by reference in their entirety.

### SEQUENCE LISTING

The instant application contains a Sequence Listing which has been submitted electronically in xml format and is hereby incorporated by reference in its entirety. Said xml copy, created on Jun. 6, 2024, is named 201953-714301-SL.xml and is 245,760 bytes in size.

### BACKGROUND

Vaccinations can provide prophylactic protection against infectious diseases, including, but not limited to, viral, bacterial, and/or parasitic diseases. For example, influenza infections are the seventh leading cause of death in the United States with 200,000 hospitalizations and 40,000 deaths seen in the United States per year and cause about 3-5 million hospitalizations and about 300,000 to 500,000 deaths worldwide per year. Millions of people receive flu vaccines to protect them from seasonal flu each year. Vaccination can also rapidly prevent the spread of an emerging influenza pandemic. Given the ability for infectious disease agents to evolve resist vaccines, there is a need for enhanced efficiency for production of vaccines and the development of vaccines with reduced changes of being evolved around by the infectious disease agents.

### BRIEF SUMMARY

The following aspects and embodiments thereof described and illustrated below are meant to be exemplary and illustrative, not limiting in scope.

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; at least one nucleic acid encoding an antigen sequence, wherein the antigen sequence comprises a sequence encoding for a viral antigen sequence, a bacterial antigen sequence, a fungal antigen sequence, or a parasitic antigen sequence, or functional variant of any of the foregoing, and wherein the viral antigen sequence is not a SARS-CoV-2 antigen sequence or functional variant thereof.

Further provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises an antigen sequence encoding for an influenza hemagglutinin protein stem region or a functional variant thereof.

Further provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises an antigen sequence encoding for a VZV protein or a functional variant thereof.

Further provided herein are compositions, wherein the compositions comprise: a lipid carrier, wherein the lipid carrier is a nanoemulsion comprising: about 30 mg/mL DOTAP chloride about 37.5 mg/mL squalene; about 37 mg/ml sorbitan monostearate; about 37 mg/ml polysorbate 80; about 10 mM sodium citrate; and about 0.2 mg Fe/ml oleic acid-coated iron oxide nanoparticles, wherein the oleic acid-coated iron oxide nanoparticle range in size from about 5 nanometers up to 25 nm, optionally, wherein the oleic acid-coated iron oxide nanoparticles are 12 nm in size; and at least one nucleic acid, wherein the at least one nucleic acid comprises a sequence at least 85% identical to SEQ ID NOS: 1-6, 38-47.

Further provided herein are compositions, wherein the compositions comprise: a lipid carrier, wherein the lipid carrier is a nanoemulsion comprising: DOTAP chloride present in an amount of about 0.75 mg; squalene present in an amount of about 0.94 mg; sorbitan monostearate present in an amount of about 0.93 mg; polysorbate 80 present in an amount of about 0.93 mg; citric acid monohydrate present in an amount of about 1.05 mg; and oleic acid-coated iron oxide nanoparticles present in an amount of about 0.005 mg; and at least one nucleic acid, wherein the at least one nucleic acid comprises a sequence at least SEQ ID NOS: 1-6, 38-47.

Further provided herein are compositions, wherein the compositions comprise (a) a lipid carrier, wherein the lipid carrier is a nanoemulsion comprising a hydrophobic core, one or more inorganic nanoparticles and one or more oils or lipids; (b) at least one nucleic acid sequence, wherein the nucleic acid sequence encodes a sequence capable of expressing an antigen, wherein the antigen is an infectious pathogen protein.

Further provided herein are dried compositions, wherein the dried compositions comprise: a lipid carrier, wherein the lipid carrier is a nanoemulsion comprising a hydrophobic core, one or more inorganic nanoparticles and one or more lipids; at least one nucleic acid sequence, wherein the nucleic acid sequence encodes a sequence capable of expressing an antigen, wherein the antigen is an infectious disease protein; and at least one cryoprotectant.

Further provided herein are vaccines comprising a composition provided herein.

Further provided herein are methods of generating an immune response in a subject, wherein the methods comprise: administering to said subject a composition provided herein. Provided herein are compositions and methods for immunoprotecting a subject comprising administering to a subject a composition provided herein.

Further provided herein are methods of reducing the severity of an infection, wherein the methods comprise: administering to a subject, prior to infection, a composition comprising: a lipid carrier, wherein the lipid carrier is a nanoemulsion comprising a hydrophobic core, one or more inorganic nanoparticles and one or more lipids; and at least one nucleic acid sequence, wherein the nucleic acid sequence encodes a sequence capable of expressing an antigen, wherein the antigen is derived from an infectious agent.

Further provided herein are kits, wherein the kits comprise a composition provided herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show schematic representations of exemplary nanoparticle (NP) carriers. FIG. 1A shows an oil-in-water emulsion. FIG. 1B shows a nanostructured lipid carrier (NLC). FIG. 1C shows a nanoparticle having an inorganic nanoparticle in liquid oil.

FIG. 2 shows anti RSV-F IgG levels in C57Bl/6 and BALB/c mice injected IM with 2.5 μg RSV repRNA formulated with a lipid carrier. Blood was collected 28 days after immunization, serum prepared and assessed by ELISA. Replicon number is indicated in parentheses (645 encodes for RSV-G and 646 encodes for RSV-F). Each point represents data from an individual animal, with the whiskers representing minimum to maximum, the box representing the interquartile range and the horizontal bar depicting the median. X-axis: repRNA RSV antigen. Y-axis: Immunoglobulin G (IgG) concentration (micrograms/milliliter).

FIGS. 3A-3B show anti-G IgG levels in C57Bl/6 and BALB/c mice injected IM with 2.5 μg RSV repRNA formulated with lipid carrier. Blood was collected 28 days after immunization, serum prepared and assessed by ELISA. Replicon number is indicated in parentheses. Each point represents data from an individual animal, with the whiskers representing minimum to maximum, the box representing the interquartile range and the horizontal bar depicting the median. X-axis: repRNA RSV antigen. Y-axis: Immunoglobulin G (IgG) concentration (micrograms/milliliter).

FIGS. 4A-4B show the increased protein production induced by the Miglyol lipid carrier formulation, NP-3. FIG. 4A shows the first assay. FIG. 4B shows the second assay.

FIGS. 5A-5B show the decreased immune response induced by the Miglyol lipid carrier formulation, NP-3. FIG. 5A shows the first assay. FIG. 5B shows the second assay.

FIGS. 6A-6B show the correlation between enhanced protein production and low TNF (e.g., TNF alpha) stimulation observed with NP-3 as a result of the first and second assays. FIG. 6A shows the first assay. FIG. 6B shows the second assay.

FIGS. 7A-7C show the effect of VZV repRNAs on antibody titers and expression. FIG. 7A shows a schematic of VZV repRNA vaccine constructs—full length glycoprotein E containing the transmembrane sequence of gE (VZV-FL-gE; black), FL-gI, gE with a truncation of the N-terminus, full length gI followed by either Thosea asigna virus 2A (T2A) ribosomal skipping peptide, or an internal ribosomal entry site (IRES) to control for co-expression of the downstream gE, and a repRNA encoding a secreted gE without the transmembrane sequence. FIG. 7B shows a Western blot and probed with anti-VZV-gE antibody. FIG. 7C shows sera antibody titers measured via ELISA against soluble VZV gE 14 days post vaccination.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION

Provided herein are compositions, kits, methods, and uses thereof for inducing an immune response to an infectious microorganism. Briefly, further described herein are (1) nanoparticle carrier systems; (2) nucleic acids encoding for microbial antigens and RNA polymerases; (3) combination compositions; (4) thermally stable, dried, and lyophilized vaccines; (5) pharmaceutical compositions; (6) dosing; (7) administration; (8) therapeutic applications; and (9) kits.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to +20%, of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve or at least partially achieve the desired effect.

### Nanoparticle Carrier Systems

Provided herein are various compositions comprising a nanoparticle or a plurality of nanoparticles. Nanoparticles also referred to herein as carriers or abbreviated as NPs. Nanoparticles provided herein may be an organic, inorganic, or a combination of inorganic and organic materials that are less than about 1 micrometer (μm) in diameter. In some embodiments, nanoparticles provided herein are used as a delivery system for a bioactive agent provided herein.

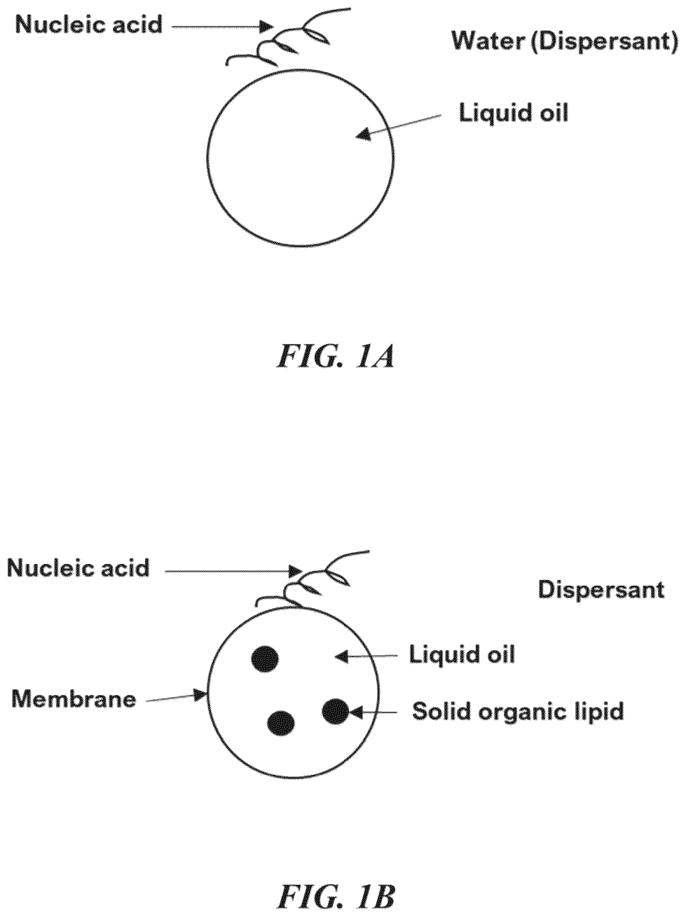

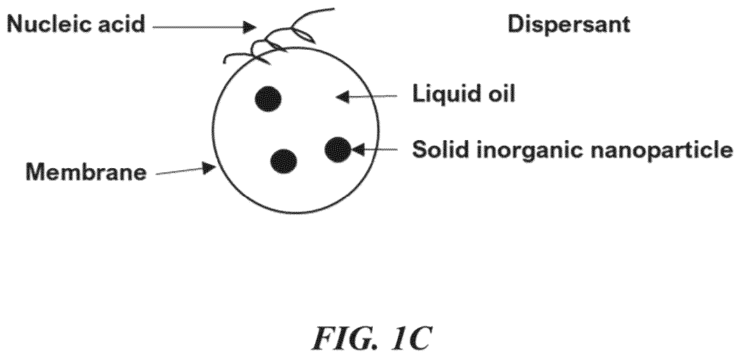

Various nanoparticles and formulations of nanoparticles (i.e., nanoemulsions) are employed. Exemplary nanoparticles are illustrated in FIGS. 1A-1C. Nanoparticles provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. The entire nanodroplet is then dispersed as a colloid in the aqueous (water) phase. In some embodiments, the nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, the nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane(DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticle provided herein comprises an oil. In some embodiments, the oil is in liquid phase. Non-limiting examples of oils that can be used include α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. In some embodiments, the nanoparticle provided herein comprises the inorganic particle within the hydrophobic core.

In some embodiments, the nanoparticle provided herein comprises a metal. In some embodiments, the nanoparticle provided herein comprises a metal within the hydrophobic core. The metal can be without limitation, a metal salt, a metal oxide, a metal hydroxide, or a metal phosphate. In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal). In some embodiments, the inorganic particle is a transition metal oxide. In some embodiments, the transition metal is magnetite (Fe₃O₄), maghemite (y-Fe₂O₃), wüstite (FeO), or hematite (alpha (α)-Fe₂O₃). In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core.

In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, the nanoparticle provided herein comprises a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant. Thus, in some embodiments, the nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85). Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5. In some embodiments, the nanoparticle provided herein comprises a hydrophilic surfactant, also called an emulsifier.

In some embodiments, the nanoparticle provided herein comprises polysorbate. Polysorbates are oily liquids derived from ethoxylated sorbitan (a derivative of sorbitol) esterified with fatty acids. In some embodiments, the nanoparticle or lipid carrier provided herein comprises a hydrophilic surfactant. Exemplary hydrophilic surfactants that can be employed include but are not limited to: polysorbates such as Tween, Kolliphor, Scattics, Alkest, or Canarcel; polyoxyethylene sorbitan ester (polysorbate); polysorbate 80 (polyoxyethylene sorbitan monooleate, or Tween 80); polysorbate 60 (polyoxyethylene sorbitan monostearate, or Tween 60); polysorbate 40 (polyoxyethylene sorbitan monopalmitate, or Tween 40); and polysorbate 20 (polyoxyethylene sorbitan monolaurate, or Tween 20). In one embodiment, the hydrophilic surfactant is polysorbate 80.

Nanoparticles provided herein comprises a hydrophobic core surrounded by a lipid membrane (e.g., a cationic lipid such as DOTAP). In some embodiments, the hydrophobic core comprises: one or more inorganic particles; a phosphate-terminated lipid; and a surfactant.

Inorganic solid nanoparticles described herein may be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises surfactant is a phosphorous-terminated surfactant, a carboxylate-terminated surfactant, a sulfate-terminated surfactant, or an amine-terminated surfactant. Typical carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate. In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

In some embodiments, a composition described herein comprises at least one nanoparticle formulations as described in Table 1. In some embodiments, a composition described herein comprises any one of NP-1 to NP-30. In some embodiments, a composition described herein comprises any one of NP-1 to NP-31. In some embodiments, the nanoparticles provided herein are admixed with a nucleic acid provided herein. In some embodiments, nanoparticles provided herein are made by homogenization and ultrasonication techniques.

TABLE 1
Nanoparticle Formulations.

| | | | | Additional |
|---|---|---|---|---|
| | | | | Ingredients |
| | Cationic Lipid(s) | Oil(s) | Surfactant(s) | %(w/v), mg/ml, |
| Name | %(w/v) or mg/ml | %(w/v) or mg/ml | %(w/v) or mg/ml | or mM |
| NP-1 | 30 mg/ml 1,2- | 37.5 mg/ml squalene | 37 mg/ml sorbitan | 0.2 mg Fe/ml 12 |
| [Fe-LC] | dioleoyl-3- | | monostearate, | nm oleic acid- |
| (LC = | trimethylammonium- | | (2R)-2- | coated iron oxide |
| lipid | propane (DOTAP) | | [(2R,3R,4S)-3,4- | nanoparticles |
| carrier) | chloride | | Dihydroxyoxolan- | 10 mM sodium |
| | | | 2-yl]-2- | citrate dihydrate. |
| | | | hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-2 | 30 mg/ml 1,2- | 37.5 mg/ml squalene | 37 mg/ml sorbitan | 1 mg Fe/ml 15 nm |
| [High Fe- | dioleoyl-3- | | monostearate, | oleic acid-coated |
| LC] | trimethylammonium- | | (2R)-2- | iron oxide |
| | propane (DOTAP) | | [(2R,3R,4S)-3,4- | nanoparticles |
| | chloride | | Dihydroxyoxolan- | 10 mM sodium |
| | | | 2-yl]-2- | citrate dihydrate. |
| | | | hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-3 | 30 mg/ml 1,2- | 37.5 mg/ml Miglyol | 37 mg/ml sorbitan | 0.2 mg Fe/ml 15 |
| [Fe-LC- | dioleoyl-3- | 812N | monostearate, | nm oleic acid- |
| Miglyol] | trimethylammonium- | (triglyceride ester of | (2R)-2- | coated iron oxide |
| | propane (DOTAP) | saturated | [(2R,3R,4S)-3,4- | nanoparticles |
| | chloride | coconut/palmkernel | Dihydroxyoxolan- | 10 mM sodium |
| | | oil derived caprylic | 2-yl]-2- | citrate dihydrate. |
| | | and capric fatty acids | hydroxyethyl | |
| | | and plant derived | octadecenoate, | |
| | | glycerol) | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-4 | 30 mg/ml 1,2- | 37.5 mg/ml Miglyol | 37 mg/ml sorbitan | 1 mg Fe/ml 15 nm |
| [High Fe- | dioleoyl-3- | 812N | monostearate, | oleic acid-coated |
| LC- | trimethylammonium- | (triglyceride ester of | (2R)-2- | iron oxide |
| Miglyol] | propane (DOTAP) | saturated | [(2R,3R,4S)-3,4- | nanoparticles |
| | chloride | coconut/palmkernel | Dihydroxyoxolan- | 10 mM sodium |
| | | oil derived caprylic | 2-yl]-2- | citrate dihydrate. |
| | | and capric fatty acids | hydroxyethyl | |
| | | and plant derived | octadecenoate, | |
| | | glycerol) | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-5 | 30 mg/ml DOTAP | 37.5 mg/ml squalene | 37 mg/ml sorbitan | 1 mg/ml |
| [Alum- | chloride | | monostearate | trioctylphosphine |
| LC] | | | (SPAN ® 60) | oxide (TOPO)- |
| | | | 37 mg/ml | coated aluminum |
| | | | polysorbate 80 | hydroxide |
| | | | (TWEEN ® 80) | (Alhydrogel ® 2%) |
| | | | | particles |
| | | | | 10 mM sodium |
| | | | | citrate dihydrate. |
| NP-6 | 30 mg/ml DOTAP | 37.5 mg/ml Solanesol | 37 mg/ml sorbitan | 0.2 mg Fe/ml oleic |
| [Fe-LC- | chloride | (Cayman chemicals), | monostearate | acid-coated iron |
| Solanesol] | | | (SPAN ® 60) | oxide |
| | | | 37 mg/ml | nanoparticles |
| | | | polysorbate 80 | 10 mM sodium |
| | | | (TWEEN ® 80) | citrate |
| NP-7 | 30 mg/ml DOTAP | 37.5 mg/ml squalene | 37 mg/ml sorbitan | 10 mM sodium |
| [NLC] | chloride | 2.4 mg/ml Dynasan | monostearate | citrate |
| | | 114 | (SPAN ® 60) | |
| | | | 37 mg/ml | |
| | | | polysorbate 80 | |
| | | | (TWEEN ® 80 | |
| NP-8 | 4 mg/ml DOTAP | 43 mg/ml squalene | 5 mg/ml Span ® | 10 mM sodium |
| [CNE] | chloride | | 85 | citrate |
| | | | 5 mg/ml Tween ® | |
| | | | 80 | |
| NP-9 | 7.5 mg/ml 1,2- | 9.4 mg/ml squalene | 9.3 mg/ml | 0.05 mg/ml 15 |
| | dioleoyl-3- | ((6E,10E,14E,18E)- | sorbitan | nanometer |
| | trimethylammonium- | 2,6,10,15,19,23- | monostearate, | superparamagnetic |
| | propane (DOTAP) | Hexamethyltetracosa- | (2R)-2- | iron oxide (Fe₃O₄) |
| | chloride | 2,6,10,14,18,22- | [(2R,3R,4S)-3,4- | 10 mM sodium |
| | | hexaene, C₃₀H₅₀) | Dihydroxyoxolan- | citrate dihydrate |
| | | 0.63 mg/ml glyceryl | 2-yl]-2- | |
| | | trimyristate-dynasan | hydroxyethyl | |
| | | (DYNASAN 114 ®) | octadecenoate, | |
| | | | C₂₄H₄₆O₆) | |
| | | | (SPAN ® 60) | |
| | | | 9.3 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-10 | 0.4% DOTAP | 0.25% glyceryl | 0.5% sorbitan | |
| | | trimyristate-dynasan | monostearate | |
| | | (DYNASAN 114 ®) | (SPAN ® 60) | |
| | | 4.75% Squalene | 0.5% polysorbate | |
| | | | 80 (TWEEN 80 ®) | |
| NP-11 | 3.0% DOTAP | 0.25% glyceryl | 3.7% | |
| | | trimyristate-dynasan | sorbitan | |
| | | (DYNASAN 114 ®) | monostearate | |
| | | 3.75% Squalene | (SPAN ® 60) | |
| | | | 3.7% polysorbate | |
| | | | 80 (TWEEN 80 ®) | |
| NP-12 | 0.4% DOTAP | 4.3% Squalene | 0.5% sorbitan | |
| | | | trioleate (SPAN ® | |
| | | | 85) | |
| | | | 0.5% polysorbate | |
| | | | 80 (TWEEN ® 80) | |
| NP-13 | 0.4% DOTAP | 0.25% glyceryl | 2.0% polysorbate | |
| | | trimyristate-dynasan | 80 (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate (SPAN ® | |
| | | (DYNASAN 114 ®) | 85) | |
| | | 4.08% squalene | 2.0% polysorbate | |
| | | | 80 (TWEEN ® 80) | |
| NP-15 | 0.4% DOTAP | 0.25% glyceryl | 0.25% sorbitan | |
| | | trimyristate-dynasan | trioleate (SPAN ® | |
| | | (DYNASAN 114 ®) | 85) | |
| | | 4.08% squalene | 2.0% polysorbate | |
| | | | 80 (TWEEN ® 80) | |
| NP-16 | 0.4% DOTAP | 5% squalene | 0.5% sorbitan | |
| | | | trioleate (SPAN ® | |
| | | | 85) | |
| | | | 2.0% polysorbate | |
| | | | 80 (TWEEN ® 80) | |
| NP-17 | 0.4% DOTAP | 5% squalene | 0.5% sorbitan | |
| | | | monostearate | |
| | | | (SPAN ® 60) | |
| | | | 2% polysorbate 80 | |
| | | | (TWEEN ® 80) | |
| NP-18 | 0.4% DOTAP | 0.25% glyceryl | 2% sorbitan | |
| | | trimyristate-dynasan | trioleate (SPAN ® | |
| | | (DYNASAN 114 ®) | 85) | |
| | | 4.08% squalene | 2% polysorbate 80 | |
| | | | (TWEEN ® 80) | |
| NP-19 | 0.4% DOTAP | 0.25% glyceryl | 0.5% sorbitan | 1% aluminum |
| | | trimyristate-dynasan | monostearate | hydroxide |
| | | (DYNASAN 114 ®) | (SPAN ® 60) | |
| | | 4.75% Squalene | 0.5% polysorbate | |
| | | | 80 (TWEEN 80 ®) | |
| NP-20 | 3.0% DOTAP | 0.25% glyceryl | 3.7% sorbitan | 1% aluminum |
| | | trimyristate-dynasan | monostearate | hydroxide |
| | | (DYNASAN 114 ®) | (SPAN ® 60) | |
| | | 3.75% Squalene | 3.7% polysorbate | |
| | | | 80 (TWEEN ® 80) | |
| NP-21 | 0.4% DOTAP | 4.3% Squalene | 0.5% sorbitan | 1% aluminum |
| | | | trioleate (SPAN ® | hydroxide |
| | | | 85) | |
| | | | 0.5% polysorbate | |
| | | | 80 (TWEEN ® 80 | |
| NP-22 | 0.4% DOTAP | 0.25% glyceryl | 2.0% polysorbate | 1% aluminum |
| | | trimyristate-dynasan | 80 (TWEEN ® 80) | hydroxide |
| | | (DYNASAN 114 ®) | | |
| | | 4.08% squalene | | |
| NP-23 | 0.4% DOTAP | 0.25% glyceryl | 0.5% sorbitan | 1% aluminum |
| | | trimyristate-dynasan | trioleate (SPAN ® | hydroxide |
| | | (DYNASAN 114 ®) | 85) | |
| | | 4.08% squalene | 2.0% polysorbate | |
| | | | 80 (TWEEN ® 80 | |
| NP-24 | 0.4% DOTAP | 0.25% glyceryl | 0.25% sorbitan | 1% aluminum |
| | | trimyristate-dynasan | trioleate (SPAN ® | hydroxide |
| | | (DYNASAN 114 ®) | 85) | |
| | | 4.08% squalene | 2.0% polysorbate | |
| | | | 80 (TWEEN ® 80 | |
| NP-25 | 0.4% DOTAP | 5% squalene | 0.5% sorbitan | 1% aluminum |
| | | | trioleate (SPAN ® | hydroxide |
| | | | 85) | |
| | | | 2.0% polysorbate | |
| | | | 80 (TWEEN ® 80 | |
| NP-26 | 0.4% DOTAP | 5% squalene | 0.5% sorbitan | 1% aluminum |
| | | | monostearate | hydroxide |
| | | | (SPAN ® 60) | |
| | | | 2% polysorbate 80 | |
| | | | (TWEEN ® 80) | |
| NP-27 | 0.4% DOTAP | 0.25% glyceryl | 2% sorbitan | 1% aluminum |
| | | trimyristate-dynasan | trioleate (SPAN ® | hydroxide |
| | | (DYNASAN 114 ®) | 85) | |
| | | 4.08% squalene | 2% polysorbate 80 | |
| | | | (TWEEN ® 80) | |
| NP-28 | 0.5-5.0 mg/ml | 0.2-10% (v/v) | 0.01-2.5% (v/v) | |
| | DOTAP | squalene | polysorbate 80 | |
| | | | (TWEEN ® 80) | |
| NP-29 | 0.4% (w/w) DOTAP | 4.3% (w/w) squalene | 0.5% (w/w) | |
| | | | sorbitan trioleate | |
| | | | (SPAN ® 85) | |
| | | | 0.5% (w/w) | |
| | | | polysorbate 80 | |
| | | | (TWEEN ® 80) | |
| NP-30 | 30 mg/ml DOTAP | 37.5 mg/ml squalene | 37 mg/ml sorbitan | 10 mM sodium |
| [LC | chloride | | monostearate | citrate |
| without | | | (SPAN ® 60) | |
| inorganic | | | 37 mg/ml | |
| core] | | | polysorbate 80 | |
| | | | (TWEEN ® 80) | |
| NP-31 | 30 mg/ml DOTAP | 37.5 mg/ml squalene | 37 mg/ml sorbitan | 0.4 mg Fe/ml 5 |
| | chloride | | monostearate | nm oleic acid- |
| | | | (SPAN ® 60) | coated iron oxide |
| | | | 37 mg/ml | nanoparticles |
| | | | polysorbate 80 | 10 mM sodium |
| | | | (TWEEN ® 80) | citrate dihydrate. |

In some embodiments, nanoparticles provided herein comprise: sorbitan monostearate (e.g., SPAN-60), polysorbate 80 (e.g., TWEEN-80), DOTAP, squalene, and no solid particles. In some embodiments, nanoparticles provided herein comprise: sorbitan monostearate (e.g., SPAN-60), polysorbate 80 (e.g., TWEEN-80), DOTAP, squalene, and iron oxide particles. In some embodiments, nanoparticles provided herein comprise an immune stimulant. In some embodiments, the immune stimulant is squalene. In some embodiments, the immune stimulant is a medium chain triglyceride. In some embodiments, the immune stimulant is Miglyol 810 or Miglyol 812. In some embodiments, the immune stimulant can decrease the total amount of protein produced, but can increase the immune response to a composition provided herein (e.g., when delivered as a vaccine). In some embodiments, the immune stimulant can increase the total amount of protein produced, but can decrease the immune response to a composition provided herein.

Nanoparticles provided herein can be of various average diameters in size. In some embodiments, nanoparticles provided herein have an average diameter (z-average hydrodynamic diameter, measured by dynamic light scattering) ranging from about 20 nm to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle) ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, the inorganic particle (e.g., iron oxide) within the hydrophobic core of the nanoparticle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm.

Nanoparticles provided herein may be characterized by the polydispersity index (PDI), which is an indication of their quality with respect to size distribution. In some embodiments, average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, the nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1. In some embodiments, the nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester. In some embodiments, nanoparticles provided herein comprise a ratio of the esters that yields a hydrophilic-lipophilic balance between 8 and 11. In some embodiments, nucleic acids provided herein are incorporated, associated with, or complexed a lipid carrier provided herein to form a lipid carrier-nucleic acid complex. In some embodiments, the lipid carrier-nucleic acid complex is formed via non-covalent interactions or via reversible covalent interactions.

### Nucleic Acids for Infectious Disease Antigen Expression

Provided herein is are compositions comprising a nanoparticle and a nucleic acid. In some embodiments, the nucleic acid is in complex with the nanoparticle. In some embodiments, the nucleic acid is in complex with the membrane of the nanoparticle. In some embodiments, the nucleic acid is in complex with the hydrophilic surface of the nanoparticle. In some embodiments, the nucleic acid is within the nanoparticle. In some embodiments, the nucleic acid is within the hydrophobic core.

In some embodiments, the nucleic acid is deoxyribonucleic acid (DNA) or ribonucleic acid (RNA). The nucleic acid may be linear or include a secondary structure (e.g., a hairpin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); ml1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); hoSU (5-hydroxyuridine); moSU (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Any one or any combination of these modified nucleobases may be included in the self-replicating RNA of the invention. Many of these modified nucleobases and their corresponding ribonucleosides are available from commercial suppliers. If desired, the nucleic acid can contain phosphoramidate, phosphorothioate, and/or methylphosphonate linkages. The RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap structure can provide stability and translational efficacy to the RNA molecule. The 5′ cap of the RNA molecule may be further modified by a 2′-O-Methyltransferase which results in the generation of a cap 1 structure (m7Gppp [m2′-O] N), which may further increase translation efficacy. A cap 1 structure may also increase in vivo potency.

In some embodiments, compositions provided herein comprise one or more nucleic acids. In some embodiments, compositions provided herein comprise two or more nucleic acids. In some embodiments, compositions provided herein comprise at least one DNA. In some embodiments, compositions provided herein comprise at least one RNA. In some embodiments, compositions provided herein comprise at least one DNA and at least one RNA. In some embodiments, nucleic acids provided herein are present in an amount of above 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of about 5 μg, about 10 μg, about 25 μg, about 50 μg, or about 100 μg. In some embodiments, nucleic acids provided herein are present in an amount of up to about 5 μg, about 10 μg, about 25 μg, about 50 μg, or 100 μg. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

### Infectious Disease Antigens

Provided herein are infectious disease antigens for recognition by hosts. In some embodiments, the infectious disease antigen is a nucleic acid encoding for an antigen protein sequence. In some embodiments, compositions provided herein comprise at least one nucleic acid sequence comprising a sequence which encodes an antigen derived from a microorganism. In some embodiments, the microorganism is an infectious microorganism. Non-limiting examples of infectious microorganisms and infectious agents include but are not limited to: viruses such as adenoviruses, herpes simplex type 1 virus, herpes simplex type 2 virus, encephalitis virus, papillomavirus, varicella-zoster virus (VZV), Epstein-Barr virus (EBV), human cytomegalovirus (CMV), Chikungunya virus, human herpes virus type 8, human papillomavirus (HPV), BK virus, JC virus, smallpox, polio virus, hepatitis B virus, human bocavirus, parvovirus B19, human astrovirus, Norwalk virus, coxsackievirus, hepatitis A virus, poliovirus, rhinovirus, Severe acute respiratory syndrome (SARS) virus, yellow fever virus, Dengue virus, West Nile virus, rubella virus, hepatitis E virus, human immunodeficiency virus (HIV), influenza virus (influenza A or influenza B), Guanarito virus, Junin virus, Lassa virus, Machupo virus, Sabii virus, Crimean-Congo hemorrhagic fever virus, Ebola virus, Marburg virus, measles virus, mumps virus, Parainfluenza virus, respiratory syncytial virus (RSV), human metapneumovirus, Hendra virus, Nipah virus, rabies virus, hepatitis D, rotavirus, orbivirus, coltivirus, banna virus, zika virus, hanta virus, West Nile virus, Middle East Respiratory Syndrome (MERS) coronavirus, Japanese encephalitis virus, and Eastern equine encephalitis; bacteria such as Acetobacter, Acinetobacter, Actinomyces, Agrobacterium, Anaplasma, Azorhizobia, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rothia, Salmonella, Shigella, Staphylococcus, Stenotrophomonas, Streptococcus, Streptococcus pneumoniae, Treponema, Vibrio, Walbachia, and Yersinia; fungi such as Aspergillus, Saccharomyces, Cryptococcus, Coccidioides, Neurospora, Histoplasma, Blastomyces; parasites such as Babesia sp., Cryptosporidium sp., Plasmodium sp., Toxoplasma sp. Plasmodium sp., Plasmodium falciparum, Plasmodium vivax, Cryptosporidium parvum, Cryptosporidium hominis, Eimeria sp., Eimeria tenella, Theileria sp., Theileria parva, Toxoplasma sp. Toxoplasma gondii, Trypanosoma brucei subspecies, Trypanosoma cruzi, Leishmania sp., and Leishmania major; and yeast such as Candida.

In some embodiments, the antigen is derived from a microorganism that causes a severe respiratory disease in mammalian populations. In some embodiments, the antigen is a surface protein or a transmembrane protein expressed on the surface of a microbial organism.

In some embodiments, the viral antigen is an influenza virus antigen. In some embodiments, the influenza virus antigen is a hemagglutinin antigen. Hemagglutinin (abbreviated HA) is a protein present on the surface of an influenza virus. On the viral surface, the hemagglutinin protein is present in homotrimers, each monomer of which is comprised of two subunits, HA1 and HA2, linked by a disulfide bond. Structurally, hemagglutinin proteins are comprised of several domains: a globular head domain, a stalk domain (also referred to as a stem or the stem protein), a transmembrane domain, and a cytoplasmic domain. Generally, during infection of a host cell (e.g., a eukaryotic cell such as a human cell) with an influenza virus, the hemagglutinin protein recognizes and binds to sialic acid of a receptor on the surface of a host cell facilitating attachment of the virus to the host cell. Following endocytosis of the virus and acidification of the endosome, the hemagglutinin protein undergoes a pH-dependent conformational change that allows for the hemagglutinin protein to facilitate fusion of the viral envelope with the endosome membrane of host cell and entry of the viral nucleic acid into the host cell. In general, influenza viruses are classified based on the amino acid sequence of the viral hemagglutinin protein and/or the amino acid sequence of the viral neuraminidase (NA). In some embodiments, nucleic acids provided herein comprise an antigen sequence encoding for an influenza hernagglutinin protein. In some embodiments, nucleic acids provided herein comprise an antigen sequence encoding for an influenza hemagglutinin protein stem region or a functional variant thereof. In some embodiments, the hemagglutinin antigen is of the subtype H1, H2, H3, H4, H5, H6, H7, H8, H9, H10, H11, H12, H13, H14, H15, H16, H17, or H18. The differences in amino acid sequences between hemagglutinin proteins of different subtypes are largely found within the sequence of the head domain of the protein. The amino acid sequence of the stem region is considered to be more conserved between hemagglutinin subtypes compared to sequence of the head domain. In some embodiments, the hemagglutinin antigen does not comprise a head domain (HA1). In some embodiments, the hemagglutinin antigen comprises a portion of the head domain (HA1). In some embodiments, the hemagglutinin antigen does not comprise a cytoplasmic domain. In some embodiments, the hemagglutinin antigen comprises a portion of the cytoplasmic domain. In some embodiments, the truncated hemagglutinin antigen. In some embodiments, the truncated hemagglutinin protein comprises a portion of the transmembrane domain. In some embodiments, the truncated hemagglutinin protein comprises a stem region or a functional fragment thereof.

In some embodiments, the viral antigen is a Varicella-Zoster Virus (VZV) antigen. In some embodiments, the VZV antigen is a glycoprotein E (gE) antigen, a glycoprotein B (gB) antigen, a glycoprotein H (gH) antigen, a glycoprotein L (gL) antigen, a glycoprotein N (gN) antigen, a glycoprotein I (gI) antigen. In some embodiments, the viral antigen is a coronavirus antigen. In some embodiments, the coronavirus is a SARS-CoV-1 coronavirus or a Middle East Respiratory Syndrome (MERS) coronavirus antigen. In some embodiments, the coronavirus antigen is a spike (S) protein antigen. In some embodiments, the viral antigen is an Epstein-Barr virus (EBV) antigen. In some embodiments, the viral antigen is a herpes simplex virus (HSV) antigen. In some embodiments, the herpes simplex virus antigen is HSV1 or HSV2 antigen. In some embodiments, the HSV antigen is a glycoprotein B, glycoprotein E, glycoprotein L, glycoprotein M, or a glycoprotein I. In some embodiments, the viral antigen is a rabies virus antigen. In some embodiments, the rabies virus antigen is a nucleoprotein (N), a phosphoprotein (P), a matrix protein (M), a glycoprotein (G) or a polymerase (L) antigen. In some embodiments, the viral antigen is a cytomegalovirus (CMV, human betaherpesvirus 5) antigen. In some embodiments, the CMV antigen is a glycoprotein B. Non-limiting examples of viral antigens for inclusion include: Zika virus envelope protein (ZIKV E), Zika virus precursor membrane and envelope proteins (prM-ENV), SARS-CoV2 spike (S) protein and envelope (E) proteins, HIV p24 antigen and Nef protein, influenza virus hemagglutinin (HA) antigen (H2, H3, H5, H6, H7, H8 and H9), influenza virus neuraminidase, rubella E1 and E2 antigens, rotavirus VP7sc antigen, RSV M2 protein, cytomegalovirus envelope glycoprotein B, the S, M, and L proteins of hepatitis B virus, rabies glycoprotein, rabies nucleoprotein, Crimean-Congo hemorrhagic fever glycoprotein Gc and or Gn, Nipah henipavirus glycoprotein, Hendra virus glycoprotein, human papillomavirus E6 protein, human papillomavirus E7 protein, human papillomavirus L1 protein, or human papillomavirus L2 protein.

In some embodiments, the antigen is a viral antigen. In some embodiments, the antigen is a respiratory syncytial virus (RSV) antigen. In some embodiments, the antigen is an RSV glycoprotein (G), RSV-G. In some embodiments, the antigen is an RSV fusion (F) glycoprotein RSV-F. In some embodiments, the antigen is a zika virus antigen. In some embodiments, the zika virus antigen is an envelope (E) protein.

In some embodiments, the antigen is a bacterial antigen. In some embodiments, the bacterial antigen is a Mycobacterium tuberculosis antigen. In some embodiments, the Mycobacterium tuberculosis antigen is H37Rv, malate synthase, or MPT51. In some embodiments, the bacterial antigen is a Chlamydia trachomatis antigen. In some embodiments, the Chlamydia trachomatis antigen is a major outer membrane protein antigen. In some embodiments, the bacterial antigen is a Staphylococcus aureus antigen.

In some embodiments, nucleic acids provided herein encodes for an antigen listed in Table 2 or a fragment thereof. In some embodiments, the nucleic acid comprises at least 80%, 85%, 90%, 95%, 96%, 97%, 98%, or 99% sequence identity to a sequence which specifically binds an antigen listed in Table 2. In some embodiments, the nucleic acid provided herein comprises at least 80%, 85%, 90%, 95%, 96%, 97%, 98%, or 99% sequence similarity to an RNA sequence listed in Table 2. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated. Exemplary nucleic acid sequences encoding for exemplary viral and bacterial antigens are listed in Table 2.

TABLE 2
Viral and Bacterial Antigens

| Infectious | | Nucleic Acid SEQ | Amino Acid SEQ ID |
|---|---|---|---|
| Microorganism | Antigen Protein | ID NO: | NOS: |
| Respiratory syncytial | RSV glycoprotein (RSV-G) | SEQ ID NO: 1 | SEQ ID NO: 7 |
| virus (RSV) (also referred | RSV fusion protein (RSV-F) | SEQ ID NO: 2 | SEQ ID NO: 8 |
| to as Human | | | (partial) |
| orthopneumovirus) | | | SEQ ID NO: 9 (full |
| | | | length) |
| Influenza virus | Haemagglutinin (HA) | SEQ ID NO: 3 | SEQ ID NO: 10 |
| Influenza A virus H3N2 | Influenza A Haemagglutinin | | SEQ ID NO: 11 |
| (New York) variant | (HA) | | |
| Influenza B | Influenza B virus HA | | SEQ ID NO: 12 |
| Haemagglutinin (HA) Lee | | | |
| 1940 Variant | | | |
| Varicella-Zoster Virus | gE | SEQ ID NO: 4 | SEQ ID NO: 13 |
| (VZV) (also referred to as | gB | | SEQ ID NO: 14 |
| Human alphaherpesvirus | gH | | SEQ ID NO: 15 |
| 3) | gL | | SEQ ID NO: 16 |
| | gN | | SEQ ID NO: 17 |
| | gI | SEQ ID NO: 5 | SEQ ID NO: 18 |
| severe acute respiratory | spike protein | | SEQ ID NO: 19 |
| syndrome coronavirus 1 | | | |
| (SARS-CoV-1) | | | |
| Epstein-Barr virus (EBV) | envelope glycoprotein B | | SEQ ID NO: 20 |
| (also referred to as human | | | |
| gammaherpesvirus 4) | | | |
| Human papilloma virus | E6 oncoprotein | | SEQ ID NO: 21 |
| (HPV) | E7 protein | | SEQ ID NO: 22 |
| Rabies virus | nucleoprotein N | | SEQ ID NO: 23 |
| | L protein | | SEQ ID NO: 24 |
| | transmembrane glycoprotein G | | SEQ ID NO: 25 |
| | phosphoprotein M1 | | SEQ ID NO: 26 |
| | M2 protein | | SEQ ID NO: 27 |
| Herpes simplex virus 1 | envelope glycoprotein B | | SEQ ID NO: 28 |
| (HSV1, also referred to as | | | |
| Human alphaherpesvirus | | | |
| 1) | | | |
| Herpes simplex virus 2 | envelope glycoprotein G | | SEQ ID NO: 29 |
| (HSV2, also referred to as | | | |
| Human alphaherpesvirus | | | |
| 2) | | | |
| Mycobacterium | Malate synthase | | SEQ ID NO: 30 |
| tuberculosis | MPT51 (H37Rv) | | SEQ ID NO: 31 |
| Zika virus | Envelope (E) protein | SEQ ID NO: 6 | SEQ ID NO: 32 |
| Chlamydia trachomatis | major outer membrane protein | | SEQ ID NO: 33 |

In some embodiments, compositions provided herein comprise a nucleic acid sequence comprising any one of SEQ ID NOS: 1-6. In some embodiments, compositions provided herein comprise a nucleic acid sequence encoding for the amino acid sequence of any one of SEQ ID NOS: 7-33.

In some embodiments, the antigen is a parasite antigen. In some embodiments the parasite antigen is a Giardia lamblia antigen, a Leishmaniasis antigen, a Plasmodium falciparum antigen, a Toxoplasma gondii antigen, a Trichomonas vaginalis antigen, a Trypanosoma brucei antigen, a Trypanosoma cruzi antigen, a Schistosoma antigen, a Toxocara antigen, a Trichinella antigen, or a Babesia antigen.

In some embodiments, the antigen is a fungal antigen. In some embodiments, the fungal antigen is a Cryptococcus antigen, an Aspergillus antigen, a Coccidioides immitis antigen, a Coccidioides posadasii antigen, a Histoplasma capsulatum antigen, a Blastomyces dermatitidis antigen, a Pneumocystis jirovecii antigen, a Trichophyton antigen, a Microsporum antigen, or a Epidermophyton antigen. In some embodiments, the antigen is a yeast antigen. In some embodiments, the yeast antigen is a Candida antigen.

### Self-Replicating Nucleic Acids

Provided herein are compositions comprising a self-replicating nucleic acid. The antigens provided herein or fragment thereof can be encoded as part of a self-replicating nucleic acid construct. In some embodiments, the self-replicating nucleic acid molecule comprises at least one or more genes selected from the group consisting of viral replicases, viral proteases, viral helicases and other nonstructural viral proteins, and also comprises 5′- and 3′-end cis-active replication sequences, and an antigenic sequence encoding an antigen protein. A subgenomic promoter that directs expression of the heterologous sequence(s) can be included in the self-replicating nucleotide sequence. If desired, a heterologous sequence may be fused in frame to other coding regions in the self-replicating RNA and/or may be under the control of an internal ribosome entry site (IRES).

In some embodiments, the self-replicating nucleotide sequence is a self-replicating RNA molecule. Self-replicating RNA molecules are designed so that the self-replicating RNA molecule cannot induce production of infectious viral particles. This can be achieved, for example, by omitting one or more viral genes encoding for structural proteins that are necessary for the production of viral particles in the self-replicating RNA. For example, when the self-replicating RNA molecule is based on an alpha virus, such as Sindbis virus (SIN), Semliki forest virus and Venezuelan equine encephalitis virus (VEE), one or more genes encoding for viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to a vertebrate cell even without any proteins, lead to the production of multiple daughter RNAs by transcription from itself (or from an antisense copy of itself). The self-replicating RNA can be directly translated after delivery to a cell, and this translation provides an RNA-dependent RNA polymerase which then produces transcripts from the delivered RNA. Thus, the delivered RNA leads to the production of multiple daughter RNAs. These transcripts are antisense relative to the delivered RNA and may be translated themselves to provide in situ expression of encoded antigens, or may be transcribed to provide further transcripts with the same sense as the delivered RNA which are translated to provide in situ expression of the encoded antigen(s).

The self-replicating RNA molecules provided herein can contain one or more modified nucleotides and therefore have improved stability and be resistant to degradation and clearance in vivo, and other advantages. In some embodiments, self-replicating RNA molecules that contain modified nucleotides avoid or reduce stimulation of endosomal and cytoplasmic immune receptors when the self-replicating RNA is delivered into a cell. This permits self-replication, amplification and expression of protein to occur. This also reduces safety concerns relative to self-replicating RNA that does not contain modified nucleotides, because the self-replicating RNA that contains modified nucleotides reduce activation of the innate immune system and subsequent undesired consequences (e.g., inflammation at injection site, irritation at injection site, pain, and the like). RNA molecules produced as a result of self-replication are recognized as foreign nucleic acids by the cytoplasmic immune receptors. Thus, self-replicating RNA molecules that contain modified nucleotides provide for efficient amplification of the RNA in a host cell and expression of antigens provided herein, as well as adjuvant effects.

In some embodiments, self-replicating RNA molecules provided herein contain at least one modified nucleotide. Modified nucleotides that are not part of the 5′ cap (e.g., in addition to the modification that are part of the 5″ cap) can be used. Accordingly, the self-replicating RNA molecule can contain a modified nucleotide at a single position, can contain a particular modified nucleotide (e.g., pseudouridine, N6-methyladenosine, 5-methylcytidine, 5-methyluridine) at two or more positions, or can contain two, three, four, five, six, seven, eight, nine, ten or more modified nucleotides (e.g., each at one or more positions). Preferably, the self-replicating RNA molecules comprise modified nucleotides that contain a modification on or in the nitrogenous base, but do not contain modified sugar or phosphate moieties. In some examples, between 0.001% and 99% or 100% of the nucleotides in a self-replicating RNA molecule are modified nucleotides. For example, 0.001%-25%, 0.01%-25%, 0.1%-25%, or 1%-25% of the nucleotides in a self-replicating RNA molecule are modified nucleotides. In other examples, between 0.001% and 99% or 100% of a particular unmodified nucleotide in a self-replicating RNA molecule is replaced with a modified nucleotide. For example, about 1% of the nucleotides in the self-replicating RNA molecule that contain uridine can be modified, such as by replacement of uridine with pseudouridine. In other examples, the desired amount (percentage) of two, three, or four particular nucleotides (nucleotides that contain uridine, cytidine, guanosine, or adenine) in a self-replicating RNA molecule are modified nucleotides. For example, 0.001%-25%, 0.01%-25%, 0.1%-25%, or 1%-25% of a particular nucleotide in a self-replicating RNA molecule are modified nucleotides. In other examples, 0.001%-20%, 0.001%-15%, 0.001%-10%, 0.01%-20%, 0.01%-15%, 0.1%-25, 0.01%-10%, 1%-20%, 1%-15%, 1%-10%, or about 5%, about 10%, about 15%, about 20% of a particular nucleotide in a self-replicating RNA molecule are modified nucleotides. It is preferred that less than 100% of the nucleotides in a self-replicating RNA molecule are modified nucleotides. It is also preferred that less than 100% of a particular nucleotide in a self-replicating RNA molecule are modified nucleotides. Thus, preferred self-replicating RNA molecules comprise at least some unmodified nucleotides.

Self-replicating RNA molecules that comprise at least one modified nucleotide can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses. Suitable synthetic methods can be used alone, or in combination with one or more other methods (e.g., recombinant DNA or RNA technology), to produce a self-replicating RNA molecule that contain one or more modified nucleotides.

Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like.

In some embodiments, nucleic acids provided herein code for an RNA polymerase. In some embodiments, nucleic acids provided herein code for a viral RNA polymerase. In some embodiments, nucleic acids provided herein code for: (1) a viral RNA polymerase; and (2) a protein or functional fragment thereof. In some embodiments, compositions provided herein comprise a first nucleic acid encoding for a viral RNA polymerase; and a second nucleic acid encoding for a protein or functional fragment thereof.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

An RNA polymerase provided herein can include but is not limited to: an alphavirus RNA polymerase, an Eastern equine encephalitis virus (EEEV) RNA polymerase, a Western equine encephalitis virus (WEEV), Venezuelan equine encephalitis virus (VEEV), Also, Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 34. In some embodiments, the nucleic acid encoding for the RNA polymerase comprises at least 90% identity to the nucleic acid sequence of SEQ ID NO: 34. In some embodiments, the nucleic acid encoding for the RNA polymerase comprises at least 95% identity to the nucleic acid sequence of SEQ ID NO: 34. In some embodiments, the nucleic acid encoding for the RNA polymerase comprises at least 99% identity to the nucleic acid sequence of SEQ ID NO: 34. In some embodiments, the nucleic acid encoding for the RNA polymerase is SEQ ID NO: 34.

In some embodiments, the amino acid sequence for VEEV RNA polymerase comprises at least 85% identity to RELPVLDSAAFNVECFKKYACNNEYWETFKENPIRLTEEN VVNYITKLKGP (SEQ ID NO: 35), TQMRELPVLDSAAFNVECFKKYACNNEYWE TFKENPIRLTE (SEQ ID NO: 36), or SEQ ID NO: 37. In some embodiments, the amino acid sequence for VEEV RNA polymerase comprises at least 90% identity to SEQ ID NO: 35, SEQ ID NO: 36, or SEQ ID NO: 37. In some embodiments, the amino acid sequence for VEEV RNA polymerase comprises at least 95% identity to SEQ ID NO: 35, SEQ ID NO: 36, or SEQ ID NO: 37. In some embodiments, the amino acid sequence for VEEV RNA polymerase comprises at least 99% identity to SEQ ID NO: 35, SEQ ID NO: 36, or SEQ ID NO: 37. In some embodiments, the amino acid sequence for VEEV RNA polymerase comprises to SEQ ID NO: 35, SEQ ID NO: 36, or SEQ ID NO: 37.

Provided herein are compositions and methods comprising replicon RNA (repRNA) encoding for one or more structural proteins from a non-enveloped virus. In some embodiments, the repRNA encodes a protease. In some embodiments, the repRNA encodes the 3CD protease. In some embodiments, the structural protein and the protease are co-expressed. In further embodiments, the repRNA comprises one or more open reading frames. In some embodiments, the open reading frames are separated by an internal ribosomal entry site (IRES). In some embodiments, the open reading frames are separated by a ribosomal skipping peptide sequence. In some embodiments the ribosomal skipping peptide sequence is from Thosea asigna virus (T2A).

### Combination Compositions

Provided herein are compositions comprising a nanoparticle described herein and a nucleic acid encoding for a coronavirus antigen. Further provided herein is a nanoemulsion comprising a plurality of nanoparticles provided herein. In some embodiments, the nucleic acid further encodes for a self-replicating RNA polymerase. In some embodiments, the nucleic acid encoding for the self-replicating RNA polymerase is on the same nucleic acid strand as the nucleic acid sequence encoding for the protein (e.g., cis). In some embodiments, the nucleic acid encoding the self-replicating RNA polymerase is on a different nucleic acid strand as the nucleic acid sequence encoding for the protein (e.g., trans). In some embodiments, the nucleic acid encoding the self-replicating RNA polymerase is a DNA molecule. In some embodiments, nucleic acid sequences encoding for an antigen provided herein are DNA or RNA molecules. In some embodiments, antigens provided herein are encoded by DNA. Nanoparticles for inclusion include, without limitation, any one of NP-1 to NP-30, or any one of NP-1 to NP-31. Nucleic acids for inclusion include, without limitation, comprise a region comprising any one of, or a plurality of, SEQ ID NOS: 1-6, 38-54 or encoding an amino acid sequence of any one of SEQ ID NOS: 7-33. In some instances, the nucleic acids further comprise a region encoding for an RNA polymerase, e.g., a region comprising a sequence of SEQ ID NO: 34.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to the antigen, increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1 In some embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In some embodiments, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene: cationic lipid, hydrophobic surfactant, and hydrophilic surfactant. In some embodiments, the oil-to-surfactant ratio is the molar ratio of squalene: DOTAP, hydrophobic surfactant, and hydrophilic surfactant. In some embodiments, the oil-to-surfactant ratio is the molar ratio of squalene: DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-cationic lipid ratio. In some embodiments, the hydrophilic surfactant-to-cationic lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1. In some embodiments, the cationic lipid is DOTAP.

Further provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN-60), polysorbate 80 (e.g., TWEEN-80), DOTAP, an immune stimulant, and an RNA.

### Thermally Stable, Dried, and Lyophilized Infectious Diseases Vaccines

Provided herein are dried or lyophilized compositions and vaccines. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

In some embodiments, a lyophilized composition is generated by a low temperature dehydration process involving the freezing of the composition, followed by a lowering of pressure, and removal of ice by sublimation. In certain cases, lyophilisation also involves the removal of bound water molecules through a desorption process. In some embodiments, compositions and vaccines provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions and vaccines provided herein at higher temperatures (e.g., greater than 4° C.) as compared to the sub-zero temperatures needed for the storage of existing mRNA vaccines. In some embodiments, dried compositions and lyophilized compositions provided herein comprise (a) a lipid carrier, wherein the lipid carrier is a nanoemulsion comprising: (i) a hydrophobic core; (ii) one or more inorganic nanoparticles; (iii) and one or more lipids; (b) one or more nucleic acids; and (c) at least one cryoprotectant. In some embodiments, the cryoprotectant is selected from the group consisting of sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions and vaccines provided herein are thermally stable. A composition is considered thermally stable when the composition resists the action of heat or cold and maintains its properties, such as the ability to protect a nucleic acid molecule from degradation at given temperature. In some embodiments, compositions and vaccines provided herein are thermally stable at about 25° C. or standard room temperature. In some embodiments, compositions and vaccines provided herein are thermally stable at about 45° C. In some embodiments, compositions and vaccines provided herein are thermally stable at about −20° C. In some embodiments, compositions and vaccines provided herein are thermally stable at about 2° C. to about 8° C. In some embodiments, compositions and vaccines provided herein are thermally stable at a temperature of at least about −80° C., at least about −20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions and vaccines provided herein are thermally stable for at least about 5 day, at least about 1 week, at least about 2 weeks, at least about 1 month, up to 3 months. In some embodiments, compositions and vaccines provided herein are stored at a temperature of at least about 4° C. up to 37° C. for at least about 5 day, at least about 1 week, at least about 2 weeks, at least about 1 month, up to 3 months. In some embodiments, compositions and vaccines provided herein are stored at a temperature of at least about 20° C. up to 25° C. for at least about 5 day, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### Pharmaceutical Compositions

Provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use. Exemplary amounts of total nucleic acid for incorporation in a composition described herein includes about 1, 2, 2.5, 5, 7.5, 10, 12.5, 15, 20, 25, 30, 35, 40, 45, 50 micrograms (μg) or more.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via a subcutaneous, intradermal, intramuscular, inhalation, intravenous, intraperitoneal, intracranial, sublingual, oral, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days.

In some embodiments, a single dose of a composition provided herein is administered to a subject. In some embodiments, a composition or pharmaceutical composition provided herein is administered to the subject by two doses. In some embodiments, a second dose of a composition or pharmaceutical composition provided herein is administered about 28 days or 56 days after the first dose. In some embodiments, a first dose is administered, and a second dose is administered about 14 days later, or about 21 days later, or about 28 days later, or about 35 days later, or about 42 days later, or about 49 days later, or about 56 days later, or about 63 days later, or about 70 days later, or about 77 days later, or about 84 days later. In some embodiments, the second dose is administered about 10-90 days following administration of the first dose, or about 15-85 days following administration of the first dose, or about 20-80 days following administration of the first dose, or about 25-75 days following administration of the first dose, or about 30-70 days following administration of the first dose, or about 35-65 days following administration of the first dose, or about 40-60 days following administration of the first dose.

In some embodiments, a third dose of a composition or pharmaceutical composition provided herein is administered to a subject. In some embodiments, the third dose is administered about 1 month following administration of the second dose, about 2 months following administration of the second dose, about 3 months following administration of the second dose, about 4 months following administration of the second dose, about 5 months following administration of the second dose, about 6 months following administration of the second dose, about 7 months following administration of the second dose, about 8 months following administration of the second dose, about 9 months following administration of the second dose, about 10 months following administration of the second dose, about 11 months following administration of the second dose, about 12 months following administration of the second dose, about 13 months following administration of the second dose, about 14 months following administration of the second dose, about 15 months following administration of the second dose, about 16 months following administration of the second dose, about 17 months following administration of the second dose, or about 18 months following administration of the second dose.

### Therapeutic Applications

Provided herein are methods of treating a disease or a disorder in a subject. Provided herein are methods of generating an immune response in a subject to an infectious microorganism. In some embodiments, the disease or disorder is an infection. In some embodiments, the infection is a viral infection, a bacterial infection, a parasitic infection, a fungal infection, or a yeast infection.

In some embodiments, the subject has, is suspected of having, or is at risk of developing a viral infection. In some embodiments, the viral infection is an RSV infection, a CMV infection, SARS, rabies, a HPV infection, chickenpox, shingles, a Herpes simplex 1 infection, a Herpes simplex 2 infection, or influenza. In some embodiments, the subject has, is suspected of having, or is at risk of developing a bacterial infection. In some embodiments, the bacterial infection is tuberculosis, chlamydia, gonorrhea, strep throat, or a Staphylococcus aureus infection. In some embodiments, the subject has or is suspected of having a fungal infection. In some embodiments, the subject has or is suspected of having a parasitic infection. In some embodiments, the parasitic infection is malaria. In some embodiments, the subject is at risk of developing an infectious disease or disorder. In some embodiments, the subject has contracted an infectious disease by way of contact with another infected subject. In some embodiments, the subject has contracted an infectious disease from contaminated drinking water. In some embodiments, the subject has contracted the infectious disease from a different species carrying the microorganism. In some embodiments, the subject is a mammal. In some embodiments, the mammal is a human.

### Kits

Provided herein is a kit comprising a composition provided herein, a pharmaceutical composition provided herein; and optionally, a delivery system for administration to a subject. In some embodiments, the kit further comprises one or more surfactants. In some embodiments, a formulation of a composition described herein is prepared in a single container for administration. In some embodiments, a formulation of a composition described herein is prepared two containers for administration, separating the nucleic acid from the nanoparticle carrier. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free. In some embodiments, the kit comprises: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises a sequence encoding for a viral antigen sequence, a bacterial antigen sequence, a fungal antigen sequence, or a parasitic antigen sequence, or functional variant thereof, wherein the viral antigen sequence is not derived from SARS-CoV-2.

### EXEMPLARY EMBODIMENTS

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; at least one nucleic acid encoding for an antigen sequence, wherein the antigen sequence comprises a sequence encoding for a viral antigen sequence, a bacterial antigen sequence, a fungal antigen sequence, or a parasitic antigen sequence, or functional variant of any of the foregoing, and wherein the viral antigen sequence is not a SARS-CoV-2 antigen sequence or functional variant thereof. Further provided herein are compositions, wherein the viral antigen sequences are derived from an influenza virus, a varicella-zoster virus (VZV), a severe acute respiratory syndrome coronavirus 1 (SARS-CoV-1), a human gammaherpesvirus 4, a human papilloma virus (HPV), a rabies virus, a human alphaherpesvirus 1, a human immunodeficiency virus (HIV), or a human alphaherpesvirus 2. Further provided herein are compositions, wherein the bacterial antigen sequences are derived from a Mycobacterium bacterium, a Streptococcus bacterium, a Pseudomonas bacterium, a Salmonella bacterium, a Staphylococcus bacterium, a Neisseria bacterium, a Clostridium bacterium, or a Chlamydia bacterium. Further provided herein are compositions, wherein the fungi antigen sequences are derived from a Aspergillus fungi, a Saccharomyces fungi, a Cryptococcus fungi, a Coccidioides fungi, a Neurospora fungi, a Histoplasma fungi, or a Blastomyces fungi. Further provided herein are compositions, wherein the parasitic antigen sequences are a derived from a Plasmodium parasite. Further provided herein are compositions, wherein the at least one nucleic acid comprises a sequence at least 85% identical to SEQ ID NOS: 1-6, 43-47. Further provided herein are compositions, wherein the at least one nucleic acid comprises a sequence encoding for an amino acid sequence of any one of SEQ ID NOS: 7-33. Further provided herein are compositions, wherein the nucleic acid is in complex with the lipid carrier. Further provided herein are compositions, wherein the nucleic acid further encodes for an RNA polymerase. Further provided herein are compositions, wherein the RNA polymerase is a Venezuelan equine encephalitis virus (VEEV) RNA polymerase. Further provided herein are compositions, wherein the nucleic acid encoding for the RNA polymerase comprises the nucleic acid sequence of SEQ ID NO: 34. Further provided herein are compositions, wherein the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier comprises a hydrophobic core. Further provided herein are compositions, wherein the lipid carrier comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the hydrophilic surfactant is a polysorbate. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the composition is lyophilized.

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises an antigen sequence encoding for an influenza hemagglutinin protein stem region or a functional variant thereof. Further provided herein are compositions, wherein the nucleic acid comprises a sequence at least 85% identical to SEQ ID NO: 3 or a functional fragment thereof. Further provided herein are compositions, wherein the nucleic acid encodes an amino acid sequence at least 85% identical to any one of SEQ ID NOS: 10-12. Further provided herein are compositions, wherein the nucleic acid is in complex with the lipid carrier. Further provided herein are compositions, wherein the nucleic acid further encodes for an RNA polymerase. Further provided herein are compositions, wherein the RNA polymerase is a Venezuelan equine encephalitis virus (VEEV) RNA polymerase. Further provided herein are compositions, wherein the nucleic acid encoding for the RNA polymerase comprises the nucleic acid sequence of SEQ ID NO: 34. Further provided herein are compositions, wherein the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier comprises a hydrophobic core. Further provided herein are compositions, wherein the lipid carrier comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the hydrophilic surfactant is a polysorbate. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are lyophilized.

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises an antigen sequence encoding for a VZV protein or a functional variant thereof. Further provided herein are compositions, wherein the VZV protein or functional variant thereof is a glycoprotein E (gE), gI, gB, gH, μL, a gN or a functional fragment thereof. Further provided herein are compositions, wherein the antigen sequence encoding for a VZV protein is at least 85% identical to any one of SEQ ID NOS: 4, 5, 38-43. Further provided herein are compositions, wherein the nucleic acid is in complex with the lipid carrier. Further provided herein are compositions, wherein the nucleic acid further encodes for an RNA polymerase. Further provided herein are compositions, wherein the RNA polymerase is a Venezuelan equine encephalitis virus (VEEV) RNA polymerase. Further provided herein are compositions, wherein the nucleic acid encoding for the RNA polymerase comprises the nucleic acid sequence of SEQ ID NO: 34. Further provided herein are compositions, wherein the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier comprises a hydrophobic core. Further provided herein are compositions, wherein the lipid carrier comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the hydrophilic surfactant is a polysorbate. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the compositions are lyophilized.

Provided herein are compositions, wherein the compositions comprise: a lipid carrier, wherein the lipid carrier is a nanoemulsion comprising: about 30 mg/mL DOTAP chloride about 37.5 mg/mL squalene; about 37 mg/ml sorbitan monostearate; about 37 mg/ml polysorbate 80; about 10 mM sodium citrate; and about 0.2 mg Fe/ml oleic acid-coated iron oxide nanoparticles, wherein the oleic acid-coated iron oxide nanoparticle range in size from about 5 nanometers up to 25 nm; and at least one nucleic acid, wherein the at least one nucleic acid comprises a sequence at least 85% identical to SEQ ID NOS: 1-6, 38-47. Further provided herein are compositions, wherein the oleic acid-coated iron oxide nanoparticles range in size from about 5 to about 25 nanometers in size. Further provided herein are compositions, wherein the oleic acid-coated iron oxide nanoparticles are 12 nanometers in size. Further provided herein are compositions, wherein the composition comprises sucrose. Further provided herein are compositions, wherein the sucrose is present in an about of about 50 mg. Further provided herein are compositions, wherein the nucleic acid is in complex with the lipid carrier. Further provided herein are compositions, wherein the nucleic acid further encodes for an RNA polymerase. Further provided herein are compositions, wherein the RNA polymerase is a Venezuelan equine encephalitis virus (VEEV) RNA polymerase. Further provided herein are compositions, wherein the nucleic acid encoding for the RNA polymerase comprises the nucleic acid sequence of SEQ ID NO: 34. Further provided herein are compositions, wherein the lipid carrier comprises a hydrophobic core. Further provided herein are compositions, wherein the oleic acid-coated iron oxide nanoparticles are within the hydrophobic core. Further provided herein are compositions, wherein the compositions are lyophilized.

Provided herein are compositions, wherein the compositions comprise: a lipid carrier, wherein the lipid carrier is a nanoemulsion comprising: DOTAP chloride present in an amount of about 0.75 mg; squalene present in an amount of about 0.94 mg; sorbitan monostearate present in an amount of about 0.93 mg; polysorbate 80 present in an amount of about 0.93 mg; citric acid monohydrate present in an amount of about 1.05 mg; and oleic acid-coated iron oxide nanoparticles present in an amount of about 0.005 mg; and at least one nucleic acid, wherein the at least one nucleic acid comprises a sequence at least SEQ ID NOS: 1-6, 38-47. Further provided herein are compositions, wherein the at least one nucleic acid sequence is present in an amount of up to about 100 micrograms (μg). Further provided herein are compositions, wherein the at least one nucleic acid sequence is present in an amount of up to about 5, about 10, about 25, about 50, or about 100 micrograms (μg). Further provided herein are compositions, wherein the at least one nucleic acid sequence is present in an amount of up to about 25 μg. Further provided herein are compositions, wherein the composition comprises sucrose. Further provided herein are compositions, wherein the sucrose is present in an about of about 50 milligrams (mg). Further provided herein are compositions, wherein the nucleic acid is in complex with the lipid carrier. Further provided herein are compositions, wherein the nucleic acid further encodes for an RNA polymerase. Further provided herein are compositions, wherein the RNA polymerase is a Venezuelan equine encephalitis virus (VEEV) RNA polymerase. Further provided herein are compositions, wherein the nucleic acid encoding for the RNA polymerase comprises the nucleic acid sequence of SEQ ID NO: 34. Further provided herein are compositions, wherein the lipid carrier comprises a hydrophobic core. Further provided herein are compositions, wherein the oleic acid-coated iron oxide nanoparticles are within the hydrophobic core. Further provided herein are compositions, wherein the compositions are lyophilized.

Provided herein are compositions, wherein the compositions comprise: a lipid carrier, wherein the lipid carrier is a nanoemulsion comprising a hydrophobic core, one or more inorganic nanoparticles and one or more oils or lipids; at least one nucleic acid sequence, wherein the nucleic acid sequence encodes a sequence capable of expressing an antigen, wherein the antigen is an infectious pathogen protein. Further provided herein are compositions, wherein the composition comprises a nucleic acid polymerase or a nucleic acid encoding for a sequence capable of expressing a nucleic acid polymerase. Further provided herein are compositions, wherein the nucleic acid sequence is an RNA nucleic acid sequence. Further provided herein are compositions, wherein the RNA polymerase or a nucleic acid sequence capable of expressing an RNA polymerase. Further provided herein are compositions, wherein the composition comprises a nucleic acid sequence comprising any one of SEQ ID NOS: 1-6, 34-54. Further provided herein are compositions, wherein the host cell is capable of expressing an antigen from a nucleic acid sequence, wherein the antigen is derived from an infectious agent. Further provided herein are compositions, wherein infectious agent is respiratory syncytial virus (RSV). Further provided herein are compositions, wherein the antigen comprises an RSV attachment (G) glycoprotein (RSV-G). Further provided herein are compositions, wherein the antigen comprises an RSV fusion (F) glycoprotein (RSV-F). Further provided herein are compositions, wherein the infectious agent is not SARS-COV-2. Further provided herein are compositions, wherein the infectious agent is a Zika virus. Further provided herein are compositions, wherein the infectious agent is an influenza virus. Further provided herein are compositions, wherein the hydrophobic core comprises an oil. Further provided herein are compositions, wherein the oil comprises at least one of α-tocopherol, lauroyl polyoxylglyceride, monoacylglycerol, propolis, squalene, squalene, miglyol, dehydroisosqualene (DHIS), mineral oil, grapeseed oil, olive oil, paraffin oil, peanut oil, soybean oil, sunflower oil, soy lecithin, triglyceride, vitamin E, and a medium chain triglyceride. Further provided herein are compositions, wherein the one or more inorganic nanoparticles is selected from the group consisting of a metal salt, metal oxide, metal hydroxide, metal phosphate, and any combinations thereof. Further provided herein are compositions, wherein the one or more lipids is selected from the group consisting of cationic lipids, anionic lipids, neutral lipids, and any combinations thereof. Further provided herein are compositions, wherein the one or more lipids is a cationic lipid. Further provided herein are compositions, wherein the cationic lipid is selected from the group consisting of 1,2-dioleoyloxy-3-(trimethylammonium)propane (DOTAP); 30-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:O)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); l,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein are compositions, wherein the lipid carrier optionally comprises one or more surfactants. Further provided herein are compositions, wherein the one or more surfactants is selected from the group consisting of hydrophobic surfactant, hydrophilic surfactant, and any combinations thereof. Further provided herein are compositions, wherein the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein are compositions, wherein the lipid carrier have a z-average hydrodynamic diameter ranging from about 40 nm to about 150 nm, with an average polydispersity index ranging from about 0.1 to about 0.4. Further provided herein are compositions, wherein the one or more nucleic acids is incorporated or complexed with the lipid carrier to form a lipid carrier-nucleic acid complex. Further provided herein are compositions, wherein the lipid carrier-RNA complex is formed via non-covalent interactions or via reversible covalent interactions. Further provided herein are compositions, wherein the molar ratio of the lipid carrier to the one or more nucleic acids, characterized by the nitrogen-to-phosphate (N:P) molar ratio, ranges from about 1:1 to about 150:1. Further provided herein are compositions, wherein the compositions are lyophilized. Further provided herein are compositions, wherein the compositions are stable at 2 to 8 degrees Celsius. Further provided herein are compositions, wherein the compositions are in the form of a suspension. Further provided herein are compositions, wherein the compositions are lyophilized. Further provided herein are vaccines comprising a composition provided herein.

Further provided herein are dried compositions, wherein the dried compositions comprise: a lipid carrier, wherein the lipid carrier is a nanoemulsion comprising a hydrophobic core, one or more inorganic nanoparticles and one or more lipids; at least one nucleic acid sequence, wherein the nucleic acid sequence encodes a sequence capable of expressing an antigen, wherein the antigen is an infectious disease protein; and at least one cryoprotectant.

Further provided herein are pharmaceutical compositions, wherein the pharmaceutical compositions comprise a composition provided herein; and a pharmaceutically acceptable excipient.

Provided herein are methods of generating an immune response in a subject, the methods comprise administering to said subject a composition provided herein. Provided herein are methods of generating an immune response in a subject, the methods comprise administering to said subject a composition provided herein, thereby generating an immune response to an antigen. Provided herein are compositions and methods for immunoprotecting a subject comprising administering to a subject a composition provided herein. Provided herein are methods of augmenting an immune response in a subject, the method comprising: administering to a subject the composition provided herein, thereby augmenting an immune response to an antigen. Provided herein are methods of treating an influenza infection, the method comprising: administering to a subject the composition provided herein thereby treating the influenza infection. Provided herein are methods of treating a VZV infection in a subject, the method comprising: administering to a subject the composition provided herein, thereby treating the VZV infection. Further provided herein are methods, wherein the compositions are administered to the subject by two doses. Further provided herein are methods, wherein the second dose is administered at about 14 days after the first dose. Further provided herein are methods, wherein the second dose is administered at about 28 days after the first dose. Further provided herein are methods, wherein the second dose is administered at about 42 days after the first dose. Further provided herein are methods, wherein the second dose is administered at about 56 days after the first dose. Further provided herein are methods, wherein the second dose is administered at about 70 days after the first dose. Further provided herein are methods, wherein the methods further comprise administering a third dose of said composition to said subject. Further provided herein are methods, wherein 5 micrograms of said composition is administered to said subject. Further provided herein are methods, wherein 10 micrograms of said composition is administered to said subject. Further provided herein are methods, wherein 25 micrograms of said composition is administered to said subject. Further provided herein are methods, wherein 30 micrograms of the compositions are administered to said subject. Further provided herein are methods, wherein 35 micrograms of the compositions are administered to said subject. Further provided herein are methods, wherein 40 micrograms of the compositions are administered to said subject. Further provided herein are methods, wherein 45 micrograms of the compositions are administered to said subject. Further provided herein are methods, wherein 50 micrograms of the compositions are administered to said subject. Further provided herein are methods, wherein 55 micrograms of the compositions are administered to said subject. Further provided herein are methods, wherein 60 micrograms of the compositions are administered to said subject. Further provided herein are methods, wherein 65 micrograms of the compositions are administered to said subject. Further provided herein are methods, wherein 70 micrograms of the compositions are administered to said subject. Further provided herein are methods, wherein 75 micrograms of the compositions are administered to said subject. Further provided herein are methods, wherein 80 micrograms of the compositions are administered to said subject. Further provided herein are methods, wherein 85 micrograms of the compositions are administered to said subject. Further provided herein are methods, wherein 90 micrograms of the compositions are administered to said subject. Further provided herein are methods, wherein 95 micrograms of the compositions are administered to said subject. Further provided herein are methods, wherein 100 micrograms of the compositions are administered to said subject. Further provided herein are methods, wherein the subject is a mammal. Further provided herein are methods, wherein the mammal is a human. Further provided herein are methods, wherein the compositions are administered intramuscularly. Further provided herein are methods, wherein the compositions are administered intranasally. the subject has, is suspected of having, or is at risk of developing a viral infection. Further provided herein are methods, wherein the viral infection is an influenza infection, a VZV infection, a severe acute respiratory syndrome infection, a human gammaherpesvirus 4 infection, a human papilloma virus (HPV) infection, a rabies virus infection, a human alphaherpesvirus 1 infection, or a human alphaherpesvirus 2 infection. Further provided herein are methods, wherein the subject has, is suspected of having, or is at risk of developing a bacterial infection. Further provided herein are methods, wherein the bacterial infection is a Mycobacterium infection, a Streptococcus infection, a Pseudomonas infection, a Salmonella infection, a Staphylococcus infection, a Neisseria infection, a Clostridium infection, or a Chlamydia infection. Further provided herein are methods, wherein the subject has, is suspected of having, or is at risk of developing a parasitic infection, a fungal infection, or a yeast infection. Further provided herein are methods, wherein the immune response comprises increasing the titer of neutralizing antibodies to the antigen as compared to a subject that has not been administered the composition. Further provided herein are methods, wherein the immune response comprises increasing the amount of CD4+ and/or CD8+ positive T cells as compared to a subject that has not been administered the composition. Further provided herein are methods, wherein the subject is immunocompromised or immunosuppressed.

Provided here are dried compositions comprising: a lipid carrier, wherein the lipid carrier is a nanoemulsion comprising a hydrophobic core, and one or more lipids; one or more nucleic acid; and at least one sugar present in amount of (i) at least about 50% by weight of the dried composition, or (ii) present in an amount of least 50 mg. Further provided herein are compositions wherein the composition is lyophilized. Further provided herein are compositions wherein the composition is thermally stable at about 25 degrees Celsius. Further provided herein are compositions wherein the composition is thermally stable at about 45 degrees Celsius. Further provided herein are compositions wherein the composition is thermally stable at about −20 degrees Celsius. Further provided herein are compositions wherein the composition is thermally stable at about 2 degrees Celsius to about 8 degrees Celsius. Further provided herein are compositions wherein the composition is thermally stable for at least 1 week, at least 2 weeks, and/or at least 1 month. Further provided herein are compositions wherein the hydrophobic core comprises an oil. Further provided herein are compositions wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, a caprylic/capric triglyceride, a triglyceride ester of saturated coconut/palmkernel oil derived caprylic and capric fatty acids and plant derived glycerol, dihydroisosqualene (DHIS), famesene and squalane. Further provided herein are compositions wherein the one or more lipids is selected from the group consisting of cationic lipids, anionic lipids, neutral lipids, and any combinations thereof. Further provided herein are compositions wherein the one or more lipids comprises a cationic lipid. Further provided herein are compositions wherein the cationic lipid is selected from the group consisting of 1,2-dioleoyloxy-3-(trimethylammonium)propane (DOTAP); 30-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein are compositions wherein the lipid carrier comprises at least one surfactant. Further provided herein are compositions wherein the at least one surfactant is selected from the group consisting of a hydrophobic surfactant, a hydrophilic surfactant, and any combinations thereof. Further provided herein are compositions wherein the hydrophobic surfactant comprises a sorbitan ester selected from the group consisting of sorbitan monolaurate, sorbitan monopalmitate, sorbitan monostearate, sorbitan tristearate, sorbitan monooleate, and sorbitan trioleate; and the hydrophilic surfactant comprises a polysorbate. Further provided herein are compositions wherein the lipid carrier has a z-average hydrodynamic diameter ranging from about 40 nm to about 150 nm, with an average polydispersity index ranging from about 0.1 to about 0.4. Further provided herein are compositions wherein the one or more nucleic acid is a DNA. Further provided herein are compositions wherein the one or more nucleic acid is a RNA. Further provided herein are compositions wherein the RNA is a self-replicating RNA. Further provided herein, are compositions wherein the hydrophobic core comprises one or more inorganic nanoparticles. Further provided herein are compositions, wherein the one or more inorganic nanoparticles is selected from the group consisting of a metal salt, metal oxide, metal hydroxide, metal phosphate, and any combinations thereof. Further provided herein are compositions wherein the one or more nucleic acid is incorporated or complexed with the lipid carrier to form a lipid carrier-nucleic acid complex. Further provided herein are compositions wherein the lipid carrier-nucleic acid complex is formed via non-covalent interactions or via reversible covalent interactions. Further provided herein are compositions wherein a molar ratio of the lipid carrier to the one or more nucleic acids, characterized by the nitrogen-to-phosphate (N:P) molar ratio, ranges from about 1:1 to about 150:1. Further provided herein are compositions wherein the at least one sugar is selected from the group consisting of sucrose, maltose, trehalose, mannitol, glucose, and any combinations thereof. Further provided herein are compositions wherein the at least one sugar is present in an amount of at least about 50, 60, 70, 80, 90, 100, 110, 120, 130, 140, 150, 160, 170, 180, 190, 200, 210, 220, 230, 240, 250, 260, 270, 280, 290, 300 or more mg. Further provided herein are compositions wherein the at least one sugar is present in an amount of 50 mg to 250 mg. Further provided herein are compositions wherein the at least one sugar is present in an amount of at least about 250 mg. Further provided herein are compositions wherein the sugar is present in amount of the composition by weight of at least 50, 55, 60, 65, 70, 75, 80, 85, 90, 95% or more. Further provided herein are compositions wherein the sugar is present in amount of the composition by weight of 80 to 98%, optionally 94 to 96%. Further provided herein are compositions wherein the sugar is present in amount of the composition by weight of about 95%. Further provided herein are compositions wherein the at least one sugar comprises sucrose. Further provided herein are pharmaceutical compositions, comprising a dried composition disclosed herein reconstituted in a suitable diluent and a pharmaceutically acceptable carrier. Further provided herein are pharmaceutical compositions wherein the diluent is aqueous. Further provided herein are pharmaceutical compositions wherein the diluent is water.

Further provided herein are kits, wherein the kits comprise a composition provided herein. Further provided herein are kits, wherein the kits comprise one or more surfactants.

### EXAMPLES

### Example 1. Immunogenicity of RSV Replicons

In order to determine the bioactivity of lipid carrier complexed with RSV repRNA and measure the in vivo generation of anti-RSV IgG, an assay was designed to first immunize 6-8 week old C57BL/6 and BALB/c female mice with the RSV repRNA and then analyze the anti-RSV target IgG levels in serum.

Initially, the formulation was prepared by combining the repRNA encoding RSV-F and G proteins (approximately 10 ng/μL, −80 degrees Celsius) and lipid carrier formulation (30 mg DOTAP/mL, 4° C.). 144 microliters of formulation was then mixed with 180 μL 40% sucrose and 36 μL 100 mM citrate and split into 2 mL tubes and inverted ten times to mix thoroughly.

Fifty microliters of the solution was injected intramuscularly into each mouse strain to yield an RNA dose of 2.5 μg on day 0, and blood was collected by retro-orbital eye bleed at day 14, 28, and 75. After collection, the blood was allowed to clot and the serum was collected and stored at −80 degrees Celsius until evaluation.

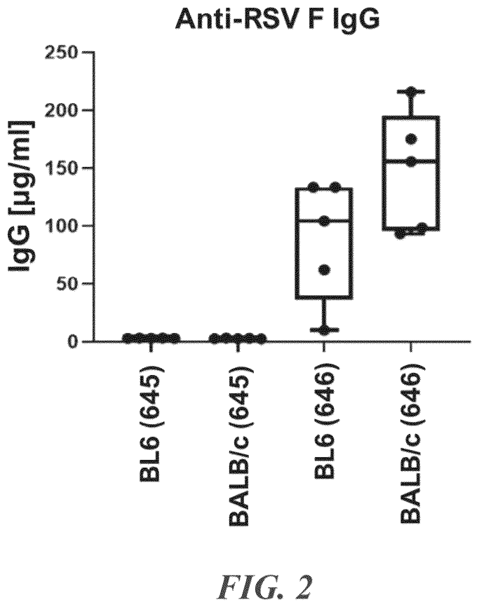

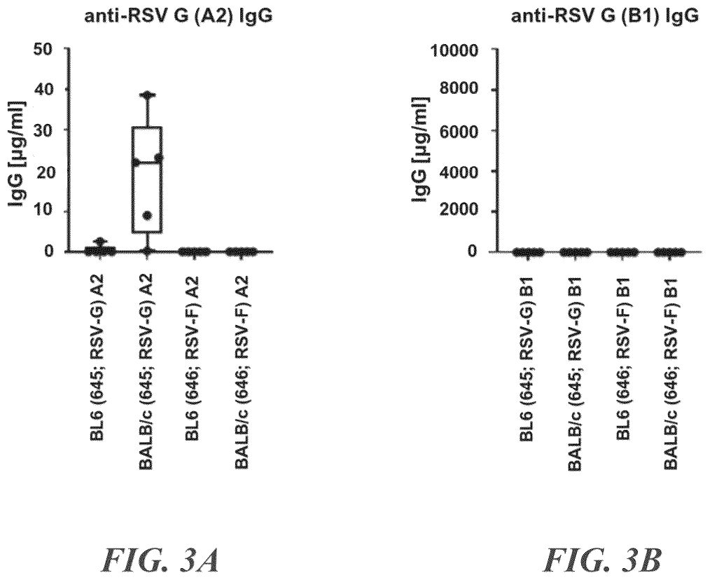

The blood collections were assessed with ELISA, using three recombinant proteins (1 μg/mL)—F Protein and G Protein with C-term His-Tag from Respiratory Syncytial Virus, B1, recombinant from RSV, and G Protein from RSV purified from HEp-2 cells. Data indicated that RSV-G protein-specific responses were induced in both C57BL/6 and BALB/c mice, while RSV-G A2 strain protein-specific responses were induced in only BALB/c mice (FIGS. 1 and 2A-2B). RSV-G B1 strain protein-specific responses were not observed, though this could have been due to an issue related to ELISA coating of RSV-G B1. RSV-G A2 protein responses were detected on the same ELISA plate, so a lack of response was not due to ELISA reagent failure.

### Example 2: Lipid Carrier Innate Immune Response in Macrophages

Various repRNA/lipid carrier formulations were prepared and analyzed in order to assess lipid carrier's innate immune response in macrophages. repRNA/lipid carrier formulations studied include Fe-lipid carrier, High Fe-lipid carrier, Fe-lipid carrier miglyol, High Fe-lipid carrier miglyol, Alum-Lipid Carrier, Fe-lipid carrier solanesol (SLN), NLC and CNE. Protein expression and stimulation of TNF production in THP-1 macrophages were studied. ELISA assay was performed to evaluate the TNF-alpha protein level in the media using the Human TNF-alpha DuoSet ELISA by R&D Systems according to the manufacturer's protocol. The 96-well microplate was coated with anti-TNF capture antibody. The plate was blocked and then media samples were added directly without dilution. After addition of the biotinylated detection antibody, SA-HRP, and substrate, the absorbance was read at 450 nm on a Spectramax i3 plate reader.

Initially, the THP-1 monocytes were differentiated into macrophages using phorbol 12-myristate 13-acetate (PMA). The cells were then transfected with various formulations with Nano Luciferase encoding replicon RNA or a PAMP. The cell culture media was then assessed for NanoLuc expression via luciferase assay and TNF expression via ELISA. The formulations used in the study are described in Table 3.

TABLE 3
Materials for Lipid Carrier Innate Immune Response Study

| Description | Concentration | Location |
|---|---|---|
| repRNA encoding NanoLuc | 909 ng/ul | −80 degrees C. |
| NP-1: Fe-Lipid carrier | 30 mg DOTAP/ml | +4 degrees C. |
| NP-2: High Fe-Lipid carrier | 30 mg DOTAP/ml | +4 degrees C. |
| NP-3: Fe-Lipid carrier miglyol | 30 mg DOTAP/ml | +4 degrees C. |
| NP-4: High Fe-Lipid carrier | 30 mg DOTAP/ml | +4 degrees C. |
| miglyol | | |
| NP-5: Alum-Lipid carrier | 30 mg DOTAP/ml | +4 degrees C. |
| NP-6: Fe-Lipid carrier | 30 mg DOTAP/ml | +4 degrees C. |
| solanesol (SLN) | | |
| NP-7: NLC | 30 mg DOTAP/ml | +4 degrees C. |
| NP-8: CNE | 4 mg DOTAP/ml | +4 degrees C. |

Eleven treatment groups were prepared. Eight of those groups were NanoLuc repRNA groups, with 600 ng repRNA dose per well was prepared using the Fe-Lipid carrier, High Fe-Lipid carrier, Fe-Lipid carrier miglyol, High Fe-Lipid carrier miglyol, Alum-Lipid carrier, Fe-Lipid carrier solanesol (SLN), NLC, and CNE formulations. Two of those groups were PAMP groups, with 200 ng dose per well prepared using the Fe-Lipid carrier and Fe-Lipid carrier miglyol formulations. The untreated group did not have NanoLuc repRNA or the PAMP.

The various formulations were prepared by diluting NanoLuc repRNA to 8 ng/μL in 2.2 mL of RNAse-free water and diluting PAMP RNA to 2.67 ng/μL in 600 μL of water. The Lipid Carrier and RNA master mix was complexed by adding 250 μL of each diluted formulation with 250 μL of diluted RNA and mixed by pipetting up and down.

Cell transfections were carried out by seeding 7×10⁵ THP-1s per well in a 24 well plate. 80 μM of PMA per well was added and incubated at 37 degrees Celsius. The next day, the PMA-containing media was removed and replaced with cRPMI for an hour before transfection. The samples were then serially diluted in Opti-MEM to make a 10-point 1.5-fold dilution series starting at 0.45 ng/μL. The culture media was then removed from the plates by pipetting. 450 μL of Opti-MEM and 150 μL of the complexed formulation was added to the plate in duplicate. The empty wells were given 450 μL of Opti-MEM only. After four hours, the samples were removed from the plate by pipetting and replaced with 500 μL of growth media. The plate was then incubated overnight at 37 degrees Celsius. The growth media was harvested the next day and stored at −80 degrees Celsius. Downstream assays were conducted.

The luciferase assay was performed by first diluting the Nano-Glo luciferase assay reagent 1:50 in buffer. 25 μL of supernatant was removed and mixed with 25 μL of Nano-Glo reagent in a 96-well plate and incubated at room temperature for 3 minutes. The luminescence was read.

ELISA assay was performed to evaluate the TNF-alpha protein level in the media using the Human TNF-alpha DuoSet ELISA by R&D Systems according to the manufacturer's protocol. The 96-well microplate was coated with anti-TNF capture antibody. The plate was blocked and then media samples were added directly without dilution. After addition of the biotinylated detection antibody, SA-HRP, and substrate, the absorbance was read at 450 nm on a Spectramax i3 plate reader.

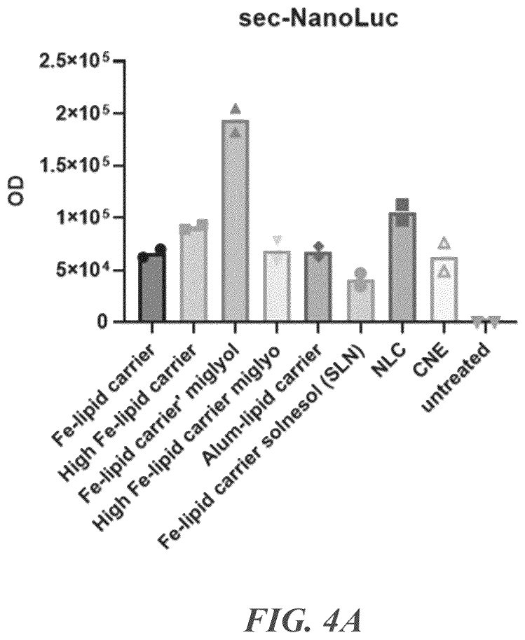

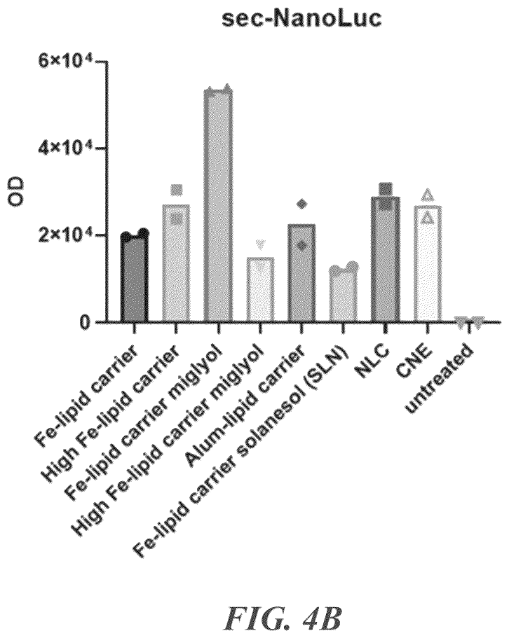

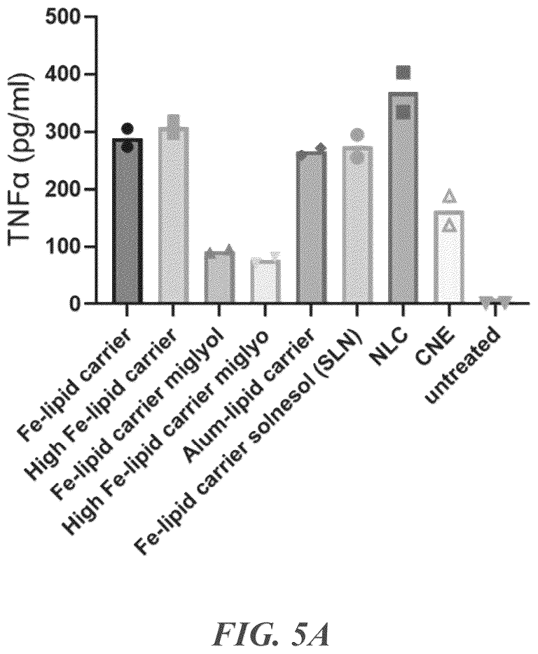

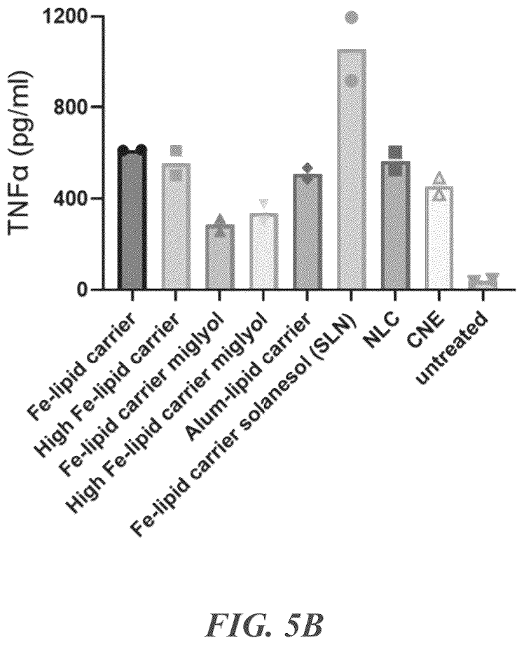

The studies described in herein demonstrate that Miglyol lipid carrier formulation induced higher protein production off the replicon, as shown in the first assay in FIG. 4A and in the second assay in FIG. 4B. A reduced innate immune response was detected, as measured by TNF-alpha secretion and is shown in the first assay in FIG. 5A and in the second assay in FIG. 5B.

Protein expression levels from the first assay in FIG. 4A and the second assay in FIG. 4B were performed using a luciferase assay by first diluting the Nano-Glo luciferase assay reagent 1:50 in buffer. 25 μL of supernatant was removed and mixed with 25 μL of Nano-Glo reagent in a 96-well plate and incubated at room temperature for 3 minutes. The luminescence was read using a luminometer.

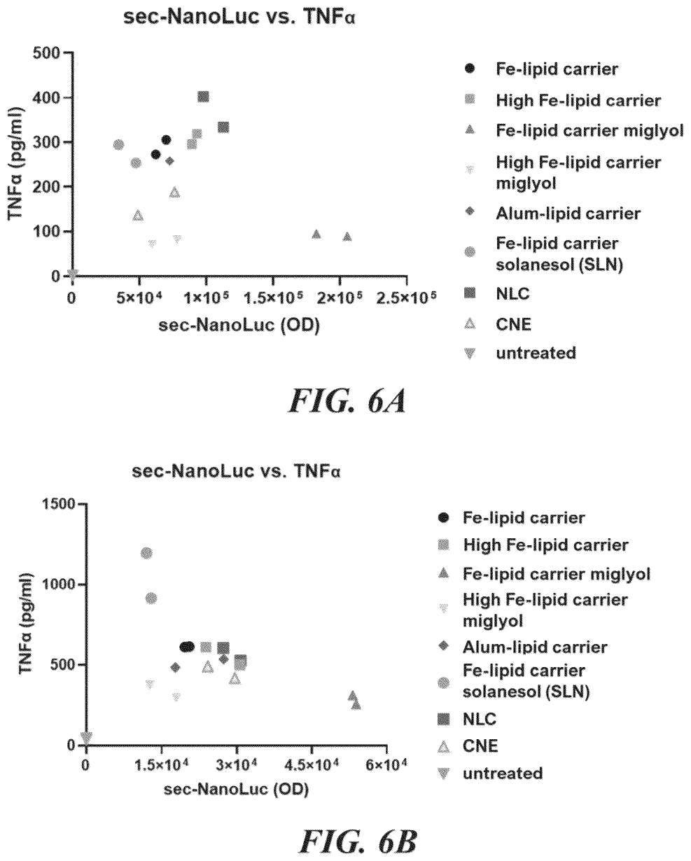

TNF-alpha protein levels in the media from the first assay in FIG. 5A and the second assay in FIG. 5B were determined using the Human TNF-alpha DuoSet ELISA by R&D Systems according to the manufacturer's protocol. The 96-well microplate was coated with anti-TNF capture antibody. The plate was blocked and then media samples were added directly without dilution. After addition of the biotinylated detection antibody, SA-HRP, and substrate, the absorbance was read at 450 nm on a Spectramax i3 plate reader. The correlation between enhanced protein production and low TNF-alpha stimulation was observed with Miglyol lipid carrier formulation, as shown in the first assay in FIG. 6A and in the second assay in FIG. 6B. The solanesol induced slightly lower protein production, but potentially higher TNF production, shown in the first assay in FIG. 6A and in the second assay in FIG. 6B. The correlation data from Assay 1 shown in FIG. 6A are derived from the data represented in FIG. 4A and FIG. 5A, and the correlation data from Assay 2 shown in FIG. 6B are derived from the data represented in FIG. 4B and FIG. 5B.

### Example 3: Manufacture and Stability of NP-1

Manufacture of NP-1. NP-1 particles comprise 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID), 0.2 mg Fe/ml 12 nm oleic acid-coated iron oxide nanoparticles (ImagionBio) and 10 mM sodium citrate dihydrate (Fisher Chemical). 1 ml of 20 mg Fe/ml 12 nm diameter oleic acid-coated iron oxide nanoparticles in chloroform (ImagionBio, lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.25 polydispersity index (PDI). The microfluidized NP-1 was terminally filtered with a 200 nm pore-size polyethersulfone (PES) filter and stored at 2-8 degrees C. Iron concentration was determined by ICP-OES. DOTAP and Squalene concentration were measured by RP-HPLC.

Manufacture of NP-3. NP-3 particles comprise 37.5 mg/ml Miglyol 812 N (IOI Oleo GmbH), 37 mg/ml Span® 60 (Millipore Sigma), 37 mg/ml Tween® 80 (Fisher Chemical), 30 mg/ml DOTAP chloride (LIPOID), 0.2 mg Fe/ml 15 nm oleic acid-coated iron oxide nanoparticles (ImagionBio) and 10 mM sodium citrate dihydrate (Fisher Chemical). 1 ml of 20 mg Fe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65 degree C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized NP-1 was terminally filtered with a 200 nm pore-size polyethersulfone (PES) filter and stored at 2-8° C. Iron concentration was determined by ICP-OES. DOTAP concentration was measured by RP-HPLC.

iii. Manufacture of NP-30. A lipid carrier without providing inorganic core particles in the core was generated having 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID) and 10 mM sodium citrate. To a 200 ml beaker 3.75 grams squalene, 3.7 grams span 60, and 3.0 grams DOTAP were added to produce the oil phase. The oil phase was sonicated for 45 minutes in a 65 degrees Celsius water bath. Separately, the aqueous phase was prepared by dissolving 19.5 grams Tween 80 in 500 ml of 10 mM sodium citrate buffer prepared in nuclease free water. 96 ml of the aqueous phase was transferred to a separate glass bottle and heated to 65 degrees Celsius for 30 minutes. The oil phase was mixed with the 96 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized NP-30 without inorganic core formulation was terminally filtered with a 200 nm pore-size polyethersulfone (PES) filter and stored at 2-8 degrees C. DOTAP and Squalene concentration were measured by RP-HPLC.

Stability. A nanoparticle according to NP-1 was placed into a stability chamber at the indicated temperatures. The stability was determined by particle size measurement using dynamic light scattering. The results show that the NP-1 formulation formed a stable colloid when stored at 4, 25 and 42 degrees Celsius. Time measurements were taken over 4 weeks. As shown in FIG. 2, the range of nanoparticle size was about 50-100 nm in diameter, and closer to 40-60 nm in diameter for the 4 and 25 degrees Celsius conditions over time.

### Example 4: Additional Nanoparticle Formulations

Additional nanoparticle formulations are produced according to the following tables (Table 4 and Table 5). The mRNA comprises a sequence encoding for an influenza viral antigen or a VZV viral antigen (SEQ ID NOS: 3-5, and 38-43, 47).

TABLE 4
mRNA Vaccine Formulation

| | Dosage form: | | | |
|---|---|---|---|---|
| | Solution for Injection for IM route of administration | | | |
| | Each 0.5 ml Vial | | | Concentration |
| | Contains: | Quantity | | (mg/ml) |
| Composition: | mRNA | 25 | mcg | 0.05 |
| | DOTAP | 0.75 | mg | 1.5 |
| | Iron Oxide Nanoparticles | 0.005 | mg | 0.01 |
| | Squalene | 0.94 | mg | 1.88 |
| | Sorbitan Monostearate | 0.93 | mg | 1.86 |
| | Polysorbate 80 | 0.93 | mg | 1.86 |
| | Sucrose IP | 50 | mg | 100 |
| | Citric Acid Monohydrate | 1.05 | mg | 2.1 |

| | |
|---|---|
| Water for Injection | q.s. to 0.5 ml |

TABLE 5
Lyophilized mRNA Vaccine Formulation

| | Dosage form: | | | | |
|---|---|---|---|---|---|
| | Lyophilized powder | | | | |
| | Each 5 dose vial | | | Concentration | Approximate dry |
| | contains: | Quantity | | (mg/ml) | weight % |
| Composition: | mRNA | 50 | mcg | 0.02 | 0.02 |
| | DOTAP | 1.5 | mg | 0.6 | 0.57 |
| | Squalene | 1.88 | mg | 0.752 | 0.72 |
| | Sorbitan Monostearate | 1.86 | mg | 0.744 | 0.71 |
| | Polysorbate 80 | 1.86 | mg | 0.744 | 0.71 |
| | Sucrose IP | 250 | mg | 100 | 95.3 |
| | Citric Acid | 5.25 | mg | 2.1 | 2 |

| Monohydrate | |
|---|---|
| Water for Injection (for | 2.5 ml |
| reconstitution) | |

### Example 5: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 34.

### Example 6: Varicella-Zoster Virus (VZV) RNA Vaccine Compositions

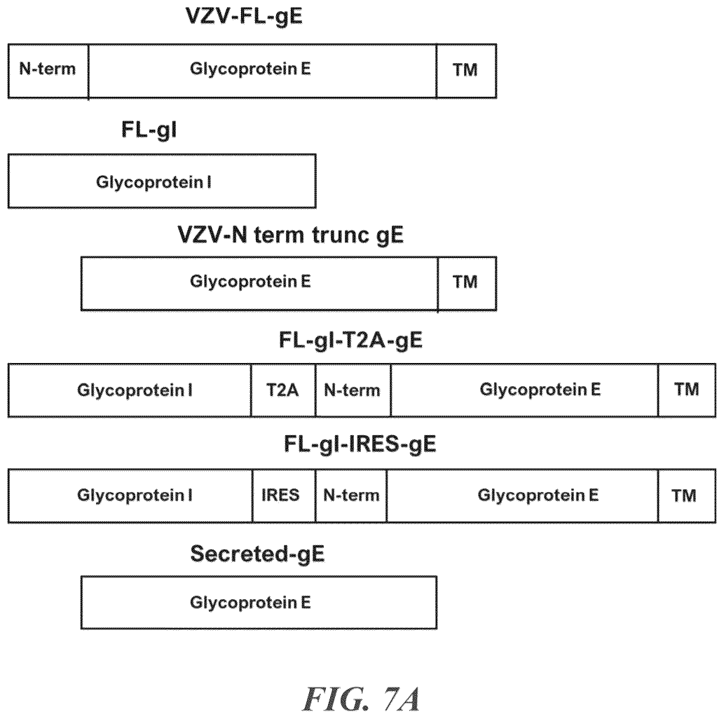

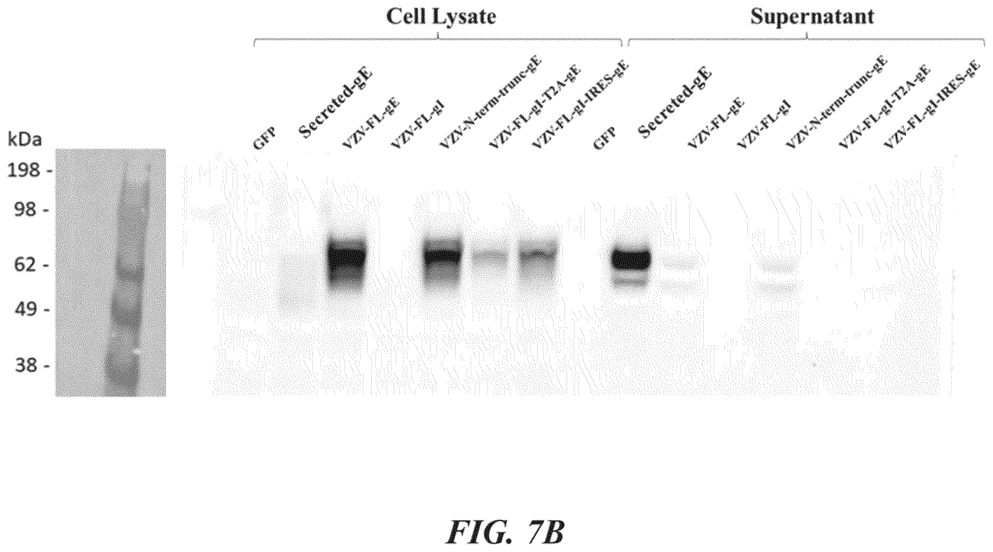

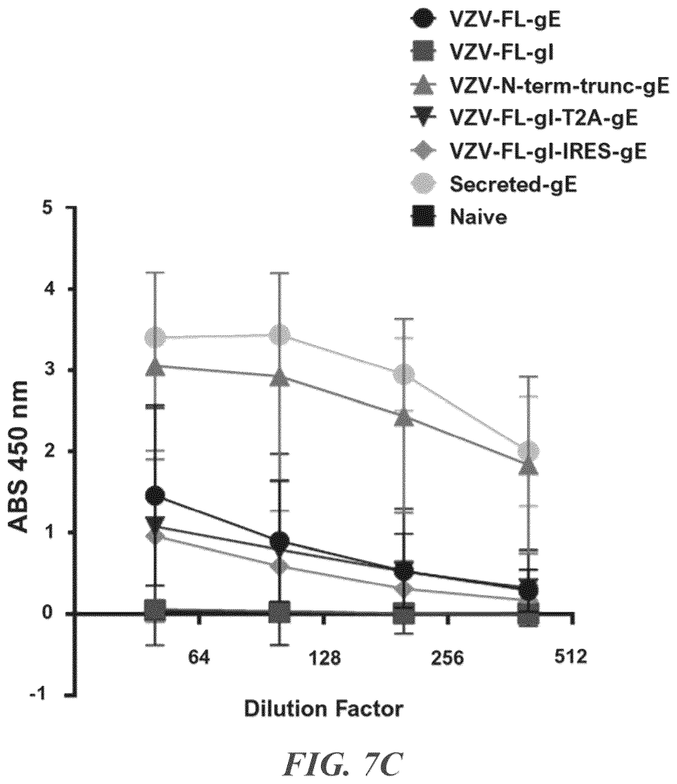

Six constructs were designed for self-replicating (repRNA) vaccines against Varicella-Zoster virus (VZV) encoding various combinations of glycoprotein E (gE) and glycoprotein I (gI). VZV gI was designedto include eitherThosea asigna virus 2A (T2A) ribosomal skipping peptide or an internal ribosomal entry site (IRES) to control for co-expression of the downstream gE. Each self-replicating RNA construct is summarized in Table 6 and illustrated in FIG. 7A.

TABLE 6
VZV- repRNA constructs

| repRNA | | RNA Sequence |
|---|---|---|
| Construct Name | Description | (SEQ ID NO:) |
| VZV-FL-gE | full length glycoprotein E containing the transmembrane | SEQ ID NO: 38 |
| | sequence of gE | |
| FL-gI | Full length glycoprotein I | SEQ ID NO: 39 |
| VZV-N term trunc | gE with a truncation of the N-terminus | SEQ ID NO: 40 |
| gE | | |
| FL-gI-T2A-gE | full length gI -Thosea asigna virus 2A (T2A)- gE | SEQ ID NO: 41 |
| FL-gI-IRES-gE | full length gI -ribosomal skipping peptide or an internal | SEQ ID NO: 42 |
| | ribosomal entry site (IRES)-gE | |
| Secreted- gE | gE without the transmembrane sequence | SEQ ID NO: 43 |

Baby hamster kidney epithelial cells (BHKs) were transfected with 4 micrograms (μg) of each corresponding RNA using lipofectamine 2000 as a delivery vehicle. Cell lysates and cell supernatants were tested via Western blot and probed with anti-VZV-gE antibody at a 1:2000 dilution (FIG. 7B). Supernatants from BHKs transfected with Secreted-gE, VZV-FL-gE, and VZV-N term trunc gE expressed anti-VZV-gE antibodies.

Six-week-old female C57BL/6 mice (n=5 mice per group) were immunized intramuscularly in the hind leg with 10 μg of repRNA encoding for a Varicella-Zoster virus (VZV) antigen (Table 6, FIG. 7A). Sera was collected 14 days post vaccination. Additionally, mice were immunized with a positive control containing repRNA encoding the gE without the transmembrane sequence, resulting in only soluble gE. Sera antibody titers were measured via ELISA against soluble VZV gE post vaccination (FIG. 7C). Mice treated with RNA encoding gE with a truncation of the N-terminus (VZV-N term trunc gE) produced increased antibody titers as compared with control mice or those immunized with the full length gE.

SEQUENCES

SEQ ID NO: 1 RSV-G Antigen Encoding RNA Sequence
auguccaaaaauaaagaccagcgaacggccaagacucuggagaggacuugggauacccugaaucaucuguuguuuau
uucaaguugccuuuauaaauugaaucucaaauccgucgcgcaaaucacgcugagcauacucgcaaugauuaucucaa
cuuccuugauaauugccgccauaaucuucauugcaagugcaaaucauaaagugacuccuacaaccgcaauaauacag
gacgcgaccagccaaauuaagaacacgacucccacguaucucacccaaaauccccaacucggaauuagcccaaguaa
uccgucagagauuacuucacaaaucaccaccauacuggcgagcacaacuccaggcguaaaauccacccuccaaucua
cuaccgucaagacuaaaaauaccacuacuacucagacacaaccuaguaaaccuacgacuaagcagcgccagaauaaa
cccccaagcaaaccaaacaacgacuuccauuuugaaguuuucaacuucgugccuuguucuaucugcucuaauaaucc
aacaugcugggccaucugcaagcgcauuccgaauaagaaacccgguaagaaaacuacaacgaaaccuaccaagaagc
cuacccugaaaacgacaaagaaagacccgaaaccgcaaacaaccaaaaguaaagaagugcccacaacuaaacccaca
gaagaaccaacgauaaacacgaccaaaaccaacauaauaacuacgcugcuuaccagcaacacgacaggcaacccgga
auugaccagucagauggaaacuuuucauucaaccagcagcgaagguaauccaaguccuagucaggugucuacgacau
cugaauauccaucucagccuaguuccccuccgaacacgccgcgccagugauaa
SEQ ID NO: 2 RSV-F Antigen Encoding RNA Sequence
auggagcugcucauccuuaaagcgaaugcuauaacuacuauacuuacagcuguaaccuuuugcuucgccucuggcca
aaacauuacugaggaguuuuaucaauccacuugcagcgcagucucaaaaggauaucugucagcauugcgcaccgggu
gguacaccagcguuaucacuaucgagcuuuccaacauuaaagagaauaaguguaacggcacagacgcgaaagucaaa
cucauuaagcaagaacuugauaaauacaaaaaugcaguuacugaacuucaguugcucaugcaaucaaccccagcaac
gaacaauagggccaggagagaauugccgagguuuaugaacuacacacuuaauaacgcuaaaaagacuaacquuacgc
ucucuaagaagcggaagcgcagguuucucggguuccuucuggggguuggcagugcaauagcauccggcgucgcggua
ucaaaggugcuucaucuugaaggagaaguuaacaaaaucaaaagcgccuugcuuucaacuaauaaggcaguaguauc
auugucuaacggugucagcgucuugacuuccaagguuuuggauuugaaaaacuauaucgacaaacaacuucucccga
ucguuaacaagcagucaugcaguaucuccaacaucgaaaccgucaucgaauuccaacagaaaaacaauagacugcuu
gaaauuacuagagaguuuucagugaacgcuggcguuaccacuccgguauccacuuauaugcucacuaauagcgaacu
gcugucucugauaaaugacaugccaauaacuaacgaccagaagaaacuuaugaguaacaacguccagaucgugagac
agcaaucauauagcauuaugagcauaauuaaggaggagguccuugcauacquaguccagcucccacuguacgggguu
aucgacacgccauguuggaagcuucauacuucccccuugugcaccacgaacacgaaggaagggucuaacauuugucu
cacgcgcacugaucgggggugguacugugacaacgccgggucagugucauuuuucccucaggccgagaccugcaagg
uccaaucaaaccggguauucugugauacuaugaacucccugacucugccuucugaaguuaaccuguguaauguagau
auauucaauccuaaauacgacugcaagauaaugaccagcaaaaccgacguguccucaucugucaucacuucccuugg
ugcuauaguaagcugcuauggcaaaacgaaaugcaccgcgaguaauaaaaaucgcgguaucaucaagacauuuagua
acggcugcgauuauguuuccaacaaagguguugacaccguaucuguggggaauacccuguauuacquaaauaagcag
gaagggaaaucucucuacgugaagggggaaccgauaaucaacuuuuaugacccgcugguuuuuccguccgacgaauu
ugacgcgaguaucucccaagucaacgagaaaauuaaccaaagccucgcguucauaagaaaauccgaugagcugcugc
auaauguaaacgcgggcaaaucaacuaccaacaucaugauuacaacaauaaucaucgugauaaucgugauccugcuu
ucacuuaucgcugucgggcuucuucucuauuguaaggcccggaguacucccgugaccuugucuaaggaucagcucuc
agguaucaacaauauugcauucagcaauuga
SEQ ID NO: 3 Influenza H3 Antigen Encoding RNA Sequence
Augaaaacaauuaucgcucucaguuauauuuucugccuuccuuuggggcaagacuugccgggcaaugauaauaguac
agcgacucucugucucggacaccacgcaguaccgaacggcacacuugugaagacaaucacagacgaucaaaucgaag
ugacgaaugcaacagagcuuguucaaucaucaucuaccggcaaaaucugcaauaauccgcauagaauccuugaugga
aucgacugcacccugauugacgcacuguugggagauccucauugcgauguguuucagaaugagacaugggaccuuuu
ugucgagaggagcaaagcguuuuccaacugcuacccguaugacgugccugauuacgcgucacuccgcucacuuguag
caucaagcgguacucuggaguucaucaccgaaggauucaccuggacgggcquaacucagaacggcgguucuaacgca
uguaagagggggccaggguccggcuucuucucacggcucaacugguugacuaagucugguucaacauacccgguccu
uaacguuacaaugccgaauaaugacaacuuugauaaacucuacaucuggggcauucaccauccauccacaaaucaag
aacagacaaguuuguacguucaggcgucagggcgcguuaccgugaguacaagaagaucacagcaaacaauuauaccc
aauauugggucccgacccuggguaagaggacuguccucucgcaucuccauauacuggaccauugucaaaccgggcga
cguccugguuaucaauaguaauggaaaccuuauugcuccgcgcggcuauuucaaaaugcgaacuggaaagucaagua
uaaugcgcucagacgcaccgaucgauacuuguaucagugaaugcaucaccccuaacggguccauaccgaacgauaag
cccuuccagaaugugaauaaaaucacguauggagcaugccccaaauacgugaagcaaaacacccucaaguuggcuac
ggguaugcgcaacgucccagaaaaacaaacgcgaggcuuguuuggggcgauagcagguuuuaucgagaacggcuggg
aaggaaugaucgaugggugguacggcuuucgccaucaaaacucagaaggaacugggcaggccgcagaucuuaagucu
acgcaagcggcgauagaucaaauuaauggcaaguugaauagggugauagagaagacgaacgagaaguuccaucaaau
agagaaagaauucagugaaguagaggggcgaauucaggauuuggaaaaauaugucgaggauacuaagaucgaccugu
ggagcuauaacgcagagcuucugguagcacuugagaaccagcauacuauugaucucaccgauuccgagaugaacaag
cuuuuugagaagaccaggagacaguugcgcgagaaugccgaagaaauggguaacgguuguuuuaagauuuaucacaa
augugacaacgcguguauugaguccauuaggaaugguacauaugaucacgauguguaucgcgaugaagcacuuaaca
aucgguuccaaauaaagggcguggagcucaagaguggguauaaagauuggauccucuggaucucauucgcgauuucc
ugcuuccuccugugcguugucuugcugggcuuuauuaugugggcaugucagagagguaacauccggugcaacauaug
uaucugauaa
SEQ ID NO: 4 Varicella-Zoster Virus (VZV) Ig E Antigen Encoding RNA Sequence
cgccaccauguucuaugaagcuuugaaggccgaacuuguauacacccgagccgugcaugguuuccgcccgagagcgaac
ugcgucguacugucagauuacauccccagagucgcgugcaacauggguacgguuaauaagcccguggugggggugcuga
uggguuuugggauaaucaccggaacucugcgcaucacaaauccuguaagagcgucagugcugagguaugacgauuucca
cauagacgaggacaagcuggauaccaauuccguuuacgaaccauauuaucacuccgaucacgccgaauccagcugggua
aaucgaggagaguccucacggaaggcauacgaccauaacuccccauauauauggccucgcaaugauuaugacggauucc
uggaaaacgcccaugagcaccacggcguuuauaaccagggucgcgguaucgauucuggggaaagguugaugcaaccaac
ucagauguccgcucaggaagauuugggggaugacaccggaauccaugugauaccuacacucaauggugaugaucgacac
aaaauuguaaacguggaccagaggcaauacggggacguuuuuaagggagaccugaauccaaaaccacaaggacaacgac
ucaucgagguuucuguagaagaaaaccauccguuuacgcuccgcgcccccauacagcgcauauauggcgugcgcuauac
ggagaccuggucuuuucuccccagucugacaugcacuggugaugcagcuccagcaauucagcacauaugucucaagcau
accaccuguuuccaagauguaguaguggauguggauugcgccgaaaacacgaaagaagaccagcuugccgagauuucau
accgguuucaaggaaaaaaagaagccgaccagccguggauaguagugaauacguccacgcucuucgaugaacuggaauu
ggacccuccugaaauugaacccggaguccugaaaguuuugcggacugagaaacaguaucucgguguguacaucuggaau
augaggggcagugauggaacaagcacauacgcgaccuuucuggugacauggaaaggugacgaaaagacuagaaacccaa
ccccagcagugaccccccaaccuagaggugcugaguuccacauguggaauuaccacucccauguauucucugucggaga
caccuucagcuuggcgaugcaccuccaauacaagauccaugaagccccguucgaccucuuguuggaguggcucuacqua
ccaauagacccuaccugucaaccaaugaggcuuuacucuacaugccuuuaccacccaaacgcaccacaaugucuuucuc
auaugaauagcgguuguacauuuacaucuccccaucucgcgcaaagagucgcgaguacaguuuaccaaaauugugagca
ugcggacaauuacacagcauauugucuugggaucucccacauggagcccucuuuugggcuuauacuucacgacgggggu
acaacgcuuaaguucgucgauacgccagaaucccucucuggcuuguauguauucgugguuuauuuuaacggucacguug
aggcuguggccuauacuguuguuagcacuguagaccacuuuguuaacgccauagaagagaggggguucccgcccaccgc
uggccagccaccggccacaacgaagccuaaagagauuacgccggugaaucccggcacaaguccccuuauccgguacgcc
gcauggacaggaggacuggcggccgucguccuccuuugucugguuauauuccuuauaugcacggcaaagcgaaugcggg
uuaaggcuuaccgaguggauaaaucuccauauaaucagagcauguauuacgcagggcucccuguggacgacuuugagga
uagcgaguccacggauacagaagaggaauuuggaaacgccaucggugggagccauggagggucaucuuacaccguuuac
auugacaaaacuagauga
SEQ ID NO: 5 Varicella-Zoster Virus (VZV) Ig I Antigen Encoding RNA Sequence
cgccaccauguuccuuauucaaugccucauauccgcagucaucuucuauauucaaguuacuaaugcacuuaucuucaag
ggcgaucaugucagccugcaagugaauucaagucuuacguccauuuugauuccaaugcagaaugauaacuauaccgaaa
uuaagggacagcugguuuuuauuggagaacaacugccgaccgguacaaacuacagcgggacccucgagcugcuuuacgc
agacacuguggcauucugcuuucggucaguacaaguuaucagauacgacggaugcccacgcauuaggacaucugccuuc
auuucuugccgauacaagcauaguuggcauuacggaaacucuaccgauagaauuucaacugaaccagaugccgguguga
ugcucaagauaaccaaaccugggaucaacgacgcagggguguauguccucuuggugagauuggaccauucaaggaguac
ggacggguuuauacugggcgugaacgucuauaccgcaggaagucaucauaacauucacggugucauuuauaccagcccc
agucuccagaauggguacagcacucgagcccuguuccagcaagcaagauugugugaccuuccagccacuccuaagggau
caggcacaagucuuuuucaacauauguuggaucucagagcagggaaaagucuugaggacaacccguggcuccaugaaga
cgugguuacuacugaaacaaagucaguggucaaggagggaaucgagaaccauguguacccaacugacaugagcacgcug
ccugaaaaaucacugaacgacccaccagagaaucugcugauaauaauaccuauuguagcgaguguuaugauuuugaccg
caauggucauaguuauuguaauaagcgugaaaaggagacgaaucaaaaaacauccgauauacaggccgaauacgaagac
aagaagagggauucagaacgcgacuccggagagugauguaaugcucgaagcagccaucgcucaacuugccaccauucgc
gaagaaagcccuccgcauuccgucguaaauccuuuugucaaguga
SEQ ID NO: 6 Zika Envelope Protein Antigen Encoding RNA Sequence
augcggagaggagcagacacauccgugggaaucgugggccugcugcugaccacagcaauggcagccgaggugaccagga
gaggcagcgccuacuauauguaccuggacagaaaugaugccggcgaggccaucuccuuucccaccacacugggcaugaa
caagugcuacauccagaucauggaccugggccacaugugcgaugccaccaugagcuaugaguguccaaugcuggacgag
ggcguggagcccgacgauguggauugcugguguaauaccacaucuacaugggugguguacggcaccugucaccacaaga
agggagaggcacggcgcagcaggagagcagugacacugccuucccacucuaccaggaagcugcagacaagaagccagac
cuggcuggaguccagggaguauacaaagcaccugaucaggguggagaacuggaucuuuagaaauccaggauucgcacug
gcugccgccgccaucgcauggcugcugggcagcuccaccagccagaaagugaucuaccuggucaugauccugcugaucg
ccccugccuauucuauccggugcaucggcgugagcaauagggacuucguggagggaauguccggaggcaccugggugga
uguggugcuggagcacggcggcugcgugacagugauggcccaggacaagccaaccguggauaucgagcuggugaccaca
accguguccaacauggccgaggugaggucuuacugcuaugaggccagcaucuccgacauggccucugauagcagauguc
ccacccagggcgaggccuaccuggacaagcaguccgauacacaguacgugugcaagcggacccugguggacaggggaug
gggaaauggauguggccuguuuggcaagggcucucuggugacaugcgccaaguucgccuguagcaagaagaugaccggc
aaguccauccagccagagaaccuggaguaccggaucaugcugucugugcacggcucccagcacucuggcaugaucguga
acgacacaggccacgagacagaugagaaucgggccaagguggagaucacaccuaacuccccacgcgccgaggccacccu
gggaggauuuggcucucugggccuggacugcgagccuaggacaggccuggacuucuccgaucuguacuaucugaccaug
aacaauaagcacuggcuggugcacaaggagugguuucacgacaucccacugccauggcacgcaggagcagauacaggca
ccccacacuggaacaauaaggaggcccugguggaguucaaggacgcccacgccaagcggcagacagugguggugcuggg
cagccaggagggagcagugcacaccgcccuggcaggcgcccuggaggccgagauggacggagcaaagggccgccugucu
agcggccaccugaagugcaggcugaagauggauaagcugagacugaagggcguguccuacucucugugcacagccgccu
ucaccuucaccaagaucccugccgagacacugcacggcacagugaccguggaggugcaguaugccggcacagacggccc
cuguaaggugccugcccagauggccguggauaugcagacacugaccccugugggcaggcugaucaccgccaauccagug
aucacagagucuaccgagaacagcaagaugaugcuggagcuggaccccccuuucggcgauagcuauaucgugaucggcg
ugggcgagaagaagaucacacaccacuggcacagaagcggcuccacaaucggcaaggccuuugaggcaaccgugcgggg
agcaaagaggauggccgugcugggcgacaccgcaugggauuucggaucugugggaggcgcccugaacagccugggcaag
ggcauccaccagaucuucggcgccgccuuuaagucccuguucggcggcaugagcugguuuucccagauccugaucggca
cacugcugauguggcugggccugaacaccaagaauggcucuaucagccugaugugccuggcccugggaggcgugcugau
cuuccuguccaccgccgugucugcc
SEQ ID NO: 7 RSV-G Amino Acid Sequence
Human respiratory syncytial virus A (strain A2) attachment glycoprotein [Human
orthopneumovirus] NCBI Reference Sequence: YP_009518856.1
MSKNKDQRTAKTLERTWDTLNHLLFISSCLYKLNLKSVAQITLSILAMIISTSLIIAAIIFIASANHKVTPTTAIIQD
ATSQIKNTTPTYLTQNPQLGISPSNPSEITSQITTILASTTPGVKSTLQSTTVKTKNTTTTQTQPSKPTTKQRQNKPP
SKPNNDFHFEVFNFVPCSICSNNPTCWAICKRIPNKKPGKKTTTKPTKKPTLKTTKKDPKPQTTKSKEVPTTKPTEEP
TINTTKTNIITTLLTSNTTGNPELTSQMETFHSTSSEGNPSPSQVSTTSEYPSQPSSPPNTPRQ
SEQ ID NO: 8 RSV-F Partial Amino Acid Sequence-F protein, partial
[Human respiratory syncytial virus B] NCBI GenBank: CBW45391.1
MELLIHRSSAIFLTLAINALYLTSSQNITEEFYQSTCSAVSRGYLSALRTGWYTSVITIELSNIKETKCNGTDTKVKL
IKQELDKYKNAVTELQLLMQNTPAANNRARREAPQYMNYTINTTKNLNVSISKKRKRRFLGFLLGVGSAIASGIAVSK
VLHLEGEVNK
SEQ ID NO: 9 RSV-F Full Length Amino Acid Sequence
[Human respiratory syncytial virus B] NCBI GenBank: QED08852.1

| | |
|---|---|
| 1 | mellihrssa ifltlainal yltssqnite efyqstcsav srgylsalrt gwytsvitie |
| 61 | lsniketkcn gtdtkvklik qeldkyknav telqllmqnt pavnnrarre apqymnytin |
| 121 | ttknlnvsis kkrkrrflgf llgvgsaias giavskvlhl egevnkikna lqstnkavvs |
| 181 | lsngvsvlts kvldlknyin nqllpivnkq scrisnietv iefqqknsrl leitrefsvn |
| 241 | agvttplsty mltnsellsl indmpitndq kklmssnvqi vrqqsysims iikeevlayv |
| 301 | vqlpiygvid tpcwklhtsp lcttnikegs nicltrtdrg wycdnagsvs ffpqadtckv |
| 361 | qsnrvfcdtm nsltlpsevs lcntdifnsk ydckimtskt disssvitsl gaivscygkt |
| 421 | kctasnknrg iiktfsngcd yvsnkgvdtv svgntlyyvn klegknlyvk gepiinyydp |
| 481 | lvfpsdefda sisqvnekin qslafirrsd ellhnvntgk sttnimitai iiviivvlls |
| 541 | liaiglllyc kakntpvtls kdqlsginni afsk |

SEQ ID NO: 10-Influenza Virus Haemagglutinin (HA) Amino Acid Sequence
hemagglutinin [Influenza A virus (A/Aichi/2/1968(H3N2))] GenBank: AAA43239.1
MKTIIALSYIFCLPLGQDLPGNDNSTATLCLGHHAVPNGTLVKTITDDQIEVTNATELVQSSSTGKICNNPHRILDGI
DCTLIDALLGDPHCDVFQNETWDLFVERSKAFSNCYPYDVPDYASLRSLVASSGTLEFITEGFTWTGVTQNGGSNACK
RGPGSGFFSRLNWLTKSGSTYPVLNVTMPNNDNFDKLYIWGIHHPSTNQEQTSLYVQASGRVTVSTRRSQQTIIPNIG
SRPWVRGLSSRISIYWTIVKPGDVLVINSNGNLIAPRGYFKMRTGKSSIMRSDAPIDTCISECITPNGSIPNDKPFQN
VNKITYGACPKYVKQNTLKLATGMRNVPEKQTRGLFGAIAGFIENGWEGMIDGWYGFRHQNSEGTGQAADLKSTQAAI
DQINGKLNRVIEKTNEKFHQIEKEFSEVEGRIQDLEKYVEDTKIDLWSYNAELLVALENQHTIDLTDSEMNKLFEKTR
RQLRENAEEMGNGCFKIYHKCDNACIESIRNGTYDHDVYRDEALNNRFQIKGVELKSGYKDWILWISFAISCFLLCVV
LLGFIMWACQRGNIRCNICI
SEQ ID NO: 11 Influenza A Haemagglutinin Amino Acid Sequence
hemagglutinin [Influenza A virus (A/New York/392/2004(H3N2))] NCBI Reference Sequence:
YP_308839.1
MKTIIALSYILCLVFAQKLPGNDNSTATLCLGHHAVPNGTIVKTITNDQIEVTNATELVQSSSTGGICDSPHQILDGE
NCTLIDALLGDPQCDGFQNKKWDLFVERSKAYSNCYPYDVPDYASLRSLVASSGTLEFNNESFNWTGVTQNGTSSACK
RRSNNSFFSRLNWLTHLKFKYPALNVTMPNNEKFDKLYIWGVHHPGTDNDQISLYAQASGRITVSTKRSQQTVIPSIG
SRPRIRDVPSRISIYWTIVKPGDILLINSTGNLIAPRGYFKIRSGKSSIMRSDAPIGKCNSECITPNGSIPNDKPFQN
VNRITYGACPRYVKQNTLKLATGMRNVPEKQTRGIFGAIAGFIENGWEGMVDGWYGFRHQNSEGTGQAADLKSTQAAI
NQINGKLNRLIGKTNEKFHQIEKEFSEVEGRIQDLEKYVEDTKIDLWSYNAELLVALENQHTIDLTDSEMNKLFERTK
KQLRENAEDMGNGCFKIYHKCDNACIGSIRNGTYDHDVYRDEALNNRFQIKGVELKSGYKDWILWISFAISCFLLCVA
LLGFIMWACQKGNIRCNICI
SEQ ID NO: 12 Influenza B Haemagglutinin Amino Acid Sequence
hemagglutinin [Influenza B virus (B/Lee/1940)] NCBI Reference Sequence: NP_056660.1
MKAIIVLLMVVTSNADRICTGITSSNSPHVVKTATQGEVNVTGVIPLTTTPTKSHFANLKGTQTRGKLCPNCFNCTDL
DVALGRPKCMGNTPSAKVSILHEVKPATSGCFPIMHDRTKIRQLPNLLRGYENIRLSTSNVINTETAPGGPYKVGTSG
SCPNVANGNGFFNTMAWVIPKDNNKTAINPVTVEVPYICSEGEDQITVWGFHSDDKTQMERLYGDSNPQKFTSSANGV
TTHYVSQIGGFPNQTEDEGLKQSGRIVVDYMVQKPGKTGTIVYQRGILLPQKVWCASGRSKVIKGSLPLIGEADCLHE
KYGGLNKSKPYYTGEHAKAIGNCPIWVKTPLKLANGTKYRPPAKLLKERGFFGAIAGFLEGGWEGMIAGWHGYTSHGA
HGVAVAADLKSTQEAINKITKNLNYLSELEVKNLQRLSGAMNELHDEILELDEKVDDLRADTISSQIELAVLLSNEGI
INSEDEHLLALERKLKKMLGPSAVEIGNGCFETKHKCNQTCLDRIAAGTFNAGDFSLPTFDSLNITAASLNDDGLDNH
TILLYYSTAASSLAVTLMIAIFIVYMVSRDNVSCSICL
SEQ ID NO: 13 VZV envelope glycoprotein E [Human alphaherpesvirus 3] GenBank: AQT34120.1
MFYEALKAELVYTRAVHGFRPRANCVVLSDYIPRVACNMGTVNKPVVGVLMGFGIITGTLRITNPVRASVLRYDDFHI
DEDKLDTNSVYEPYYHSDHAESSWVNRGESSRKAYDHNSPYIWPRNDYDGFLENAHEHHGVYNQGRGIDSGERLMQPT
QMSAQEDLGDDTGIHVIPTLNGDDRHKIVNVDQRQYGDVFKGDLNPKPQGQRLIEVSVEENHPFTLRAPIQRIYGVRY
TETWSFLPSLTCTGDAAPAIQHICLKHTTCFQDVVVDVDCAENTKEDQLAEISYRFQGKKEADQPWIVVNTSTLFDEL
ELDPPEIEPGVLKVLRTEKQYLGVYIWNMRGSDGTSTYATFLVTWKGDEKTRNPTPAVTPQPRGAEFHMWNYHSHVFS
VGDTFSLAMHLQYKIHEAPFDLLLEWLYVPIDPTCQPMRLYSTCLYHPNAPQCLSHMNSGCTFTSPHLAQRVASTVYQ
NCEHADNYTAYCLGISHMEPSFGLILHDGGTTLKFVDTPESLSGLYVFVVYFNGHVEAVAYTVVSTVDHFVNAIEERG
FPPTAGQPPATTKPKEITPVNPGTSPLIRYAAWTGGLAAVVLLCLVIFLICTAKRMRVKAYRVDKSPYNQSMYYAGLP
VDDFEDSESTDTEEEFGNAIGGSHGGSSYTVYIDKTR
SEQ ID NO: 14 VZV envelope glycoprotein B [Human alphaherpesvirus 3] NCBI Reference
Sequence: NP_040154.2
MSPCGYYSKWRNRDRPEYRRNLRFRRFFSSIHPNAAAGSGFNGPGVFITSVTGVWLCFLCIFSMFVTAVVSVSPSSFY
ESLQVEPTQSEDITRSAHLGDGDEIREAIHKSQDAETKPTFYVCPPPTGSTIVRLEPTRTCPDYHLGKNFTEGIAVVY
KENIAAYKFKATVYYKDVIVSTAWAGSSYTQITNRYADRVPIPVSEITDTIDKFGKCSSKATYVRNNHKVEAFNEDKN
PQDMPLIASKYNSVGSKAWHTTNDTYMVAGTPGTYRTGTSVNCIIEEVEARSIFPYDSFGLSTGDIIYMSPFFGLRDG
AYREHSNYAMDRFHQFEGYRQRDLDTRALLEPAARNFLVTPHLTVGWNWKPKRTEVCSLVKWREVEDVVRDEYAHNFR
FTMKTLSTTFISETNEFNLNQIHLSQCVKEEARAIINRIYTTRYNSSHVRTGDIQTYLARGGFVVVFQPLLSNSLARL
YLQELVRENTNHSPQKHPTRNTRSRRSVPVELRANRTITTTSSVEFAMLQFTYDHIQEHVNEMLARISSSWCQLQNRE
RALWSGLFPINPSALASTILDQRVKARILGDVISVSNCPELGSDTRIILQNSMRVSGSTTRCYSRPLISIVSLNGSGT
VEGQLGTDNELIMSRDLLEPCVANHKRYFLFGHHYVYYEDYRYVREIAVHDVGMISTYVDLNLTLLKDREFMPLQVYT
RDELRDTGLLDYSEIQRRNQMHSLRFYDIDKVVQYDSGTAIMQGMAQFFQGLGTAGQAVGHVVLGATGALLSTVHGFT
TFLSNPFGALAVGLLVLAGLVAAFFAYRYVLKLKTSPMKALYPLTTKGLKQLPEGMDPFAEKPNATDTPIEEIGDSQN
TEPSVNSGFDPDKFREAQEMIKYMTLVSAAERQESKARKKNKTSALLTSRLTGLALRNRRGYSRVRTENVTGV
SEQ ID NO: 15 VZV envelope glycoprotein H [Human alphaherpesvirus 3] GenBank: QXN54893.1
MFALVLAVVILPLWTTANKSYVTPTPATRSIGHMSALLREYSDRNMSLKLEAFYPTGFDEELIKSLHWGNDRKHVFLV
IVKVNPTTHEGDVGLVIFPTYLLSPYHFKAEHRAPFPAGRFGFLSHPVTPDVSFFDSSFAPYLTTQHLVAFTTFPPNP
LVWHLERAETAATAERPFGVSLLPARPTVPKNTILEHKAHFATWDALARHTFFSAEAIITNSTLRIHVPLFGSVWPIR
YWATGSVLLTSDSGRVEVNIGVGFMSSLISLSSGLPIELIVVPHTVKLNAVTSDTTWFQLNPPGPDPGPSYRVYLLGR
GLDMNFSKHATVDICAYPEESLDYRYHLSMAHTEALRMTTKADQHDINEESYYHIAARIATSIMGRTTEYFLLDEIVD
VQYQLKFLNYILMRIGAGAHPNTISGTSDLIFADPSQLHDELSLLFGQVKPANVDYFISYDEARDQLKTAYALSRGQD
HVNALSLARRVIMSIYKGLLVKQNLNATERQALFFASMILLNFREGLENSSRVLDGRTTLLLMTSMCTAAHATQAALN
IQEGLAYLNPSKHMFTIPNVYSPCMGSLRTDLTEEIHVMNLLSAIPTRPGLNEVLHTQLDESEIFDAAFKTMMIFTTW
TAKDLHILHTHVPEVFTCQDAAARNGEYVLILPAVQGHSYVITRNKPQRGLVYSLADVDVYNPISVVYLSKDTCVSEH
GVIETVALPHPDNLKECLYCGSVFLRYLTTGAIMDIIIIDSKDTERQLAAMGNSTIPPFNPDMHGDDSKAVLLFPNGT
VVTLLGFERRQAIRMSGQYLGASLGGAFLAVVGFGIIGWMLCGNSRLREYNKIPLT
SEQ ID NO: 16 VZV glycoprotein gL [Human alphaherpesvirus 3] GenBank: ABE03078.1
MASHKWLLQMIVFLKTITIAYCLHLQDDTPLFFGAKPLSDVSLIITEPCVSSVYEAWDYAAPPVSNLSEALSGIVVKT
KCPVPEVILWFKDKQMAYWTNPYVTLKGLAQSVGEEHKSGDIRDALLDALSGVWVDSTPSSTNIPENGCVWGADRLFQ
RVCQ
SEQ ID NO: 17 VZV envelope glycoprotein N [Human alphaherpesvirus 3] GenBank: QXN54863.1
MGSITASFILITMQILFFCEDSSGEPNFAERNFWHASCSARGVYIDGSMITTLFFYASLLGVCVALISLAYHACFRLF
TRSVLRSTW
SEQ ID NO: 18 VZV envelope glycoprotein I [Human alphaherpesvirus 3] Genbank: QXN54922.1
MFLIQCLISAVIFYIQVTNALIFKGDHVSLQVNSSLTSILIPMQNDNYTEIKGQLVFIGEQLPTGTNYSGTLELLYAD
TVAFCFRSVQVIRYDGCPRIRTSAFISCRYKHSWHYGNSTDRISTEPDAGVMLKITKPGINDAGVYVLLVRLDHSRST
DGFILGVNVYTAGSHHNIHGVIYTSPSLQNGYSTRALFQQARLCDLPATPKGSGTSLFQHMLDLRAGKSLEDNPWLHE
DVVTTETKSVVKEGIENHVYPTDMSTLPEKSINDPPENLLIIIPIVASVMILTAMVIVIVISVKRRRIKKHPIYRPNT
KTRRGIQNATPESDVMLEAAIAQLATIREESPPHSVVNPFVK
SEQ ID NO: 19 spike glycoprotein [Severe acute respiratory syndrome-related
coronavirus-1] GenBank: ABD73002.1
MFIFLLFLTLTSGSDLDRCTTFDDVQAPNYTQHTSSMRGVYYPDEIFRSDTLYLTQDLFLPFYSNVTGFHTINHTFGN
PVIPFKDGIYFAATEKSNVVRGWVFGSTMNNKSQSVIIINNSTNVVIRACNFELCDNPFFAVSKPMGTQTHTMIFDNA
FNCTFEYISDAFSLDVSEKSGNFKHLREFVFKNKDGFLYVYKGYQPIDVVRDLPSGFNTLKPIFKLPLGINITNFRAI
LTAFSPAQDIWGTSAAAYFVGYLKPTTFMLKYDENGTITDAVDCSQNPLAELKCSVKSFEIDKGIYQTSNFRVVPSGD
VVRFPNITNLCPFGEVFNATKFPSVYAWERKKISNCVADYSVLYNSTFFSTFKCYGVSATKLNDLCFSNVYADSFVVK
GDDVRQIAPGQTGVIADYNYKLPDDFMGCVLAWNTRNIDATSTGNYNYKYRYLRHGKLRPFERDISNVPFSPDGKPCT
PPALNCYWPLNDYGFYTTTGIGYQPYRVVVLSFELLNAPATVCGPKLSTDLIKNQCVNFNFNGLTGTGVLTPSSKRFQ
PFQQFGRDVSDFTDSVRDPKTSEILDISPCSFGGVSVITPGTNASSEVAVLYQDVNCTDVSTAIHADQLTPAWRIYST
GNNVFQTQAGCLIGAEHVDTSYECDIPIGAGICASYHTVSLLRSTSQKSIVAYTMSLGADSSIAYSNNTIAIPTNFSI
SITTEVMPVSMAKTSVDCNMYICGDSTECANLLLQYGSFCTQLNRALSGIAAEQDRNTREVFAQVKQMYKTPTLKYFG
GFNFSQILPDPLKPTKRSFIEDLLFNKVTLADAGFMKQYGECLGDINARDLICAQKFNGLTVLPPLLTDDMIAAYTAA
LVSGTATAGWTFGAGAALQIPFAMQMAYRFNGIGVTQNVLYENQKQIANQFNKAISQIQESLTTTSTALGKLQDVVNQ
NAQALNTLVKQLSSNFGAISSVLNDILSRLDKVEAEVQIDRLITGRLQSLQTYVTQQLIRAAEIRASANLAATKMSEC
VLGQSKRVDFCGKGYHLMSFPQAAPHGVVFLHVTYVPSQERNFTTAPAICHEGKAYFPREGVFVFNGTSWFITQRNFF
SPQIITTDNTFVSGNCDVVIGIINNTVYDPLQPELDSFKEELDKYFKNHTSPDVDLGDISGINASVVNIQKEIDRLNE
VAKNLNESLIDLQELGKYEQYIKWPWYVWLGFIAGLIVIVMVTILLCCMTSCCSCLKGACSCGSCCKFDEDDSEPVLK
GVKLHYT
SEQ ID NO: 20 envelope glycoprotein B [Human gammaherpesvirus 4]
GenBank: QXN54824.1
MTRRRVLSVVVLLAALACRLGAQTPEQPAPPATTVQPTATRQQTSFPFRVCELSSHGDLFRFSSDIQCPSFGTRENHT
EGLLMVFKDNIIPYSFKVRSYTKIVTNILIYNGWYADSVTNRHEEKFSVDSYETDQMDTIYQCYNAVKMTKDGLTRVY
VDRDGVNITVNLKPTGGLANGVRRYASQTELYDAPGWLIWTYRTRTTVNCLITDMMAKSNSPFDFFVTTTGQTVEMSP
FYDGKNKETFHERADSFHVRTNYKIVDYDNRGTNPQGERRAFLDKGTYTLSWKLENRTAYCPLQHWQTFDSTIATETG
KSIHFVTDEGTSSFVTNTTVGIELPDAFKCIEEQVNKTMHEKYEAVQDRYTKGQEAITYFITSGGLLLAWLPLTPRSL
ATVKNLTELTTPTSSPPSSPSPPAPSAARGSTPAAVLRRRRRDAGNATTPVPPTAPGKSLGTLNNPATVQIQFAYDSL
RRQINRMLGDLARAWCLEQKRQNMVLRELTKINPTTVMSSIYGKAVAAKRLGDVISVSQCVPVNQATVTLRKSMRVPG
SETMCYSRPLVSFSFINDTKTYEGQLGTDNEIFLTKKMTEVCQATSQYYFQSGNEIHVYNDYHHFKTIELDGIATLQT
FISLNTSLIENIDFASLELYSRDEQRASNVFDLEGIFREYNFQAQNIAGLRKDLDNAVSNGRNQFVDGLGELMDSLGS
VGQSITNLVSTVGGLFSSLVSGFISFFKNPFGGMLILVLVAGVVILVISLTRRTRQMSQQPVQMLYPGIDELAQQHAS
GEGPGINPISKTELQAIMLALHEQNQEQKRAAQRAAGPSVASRALQAARDRFPGLRRRRYHDPETAAALLGEAETEF
SEQ ID NO: 21 E6 oncoprotein [Human papillomavirus] GenBank: QNJ45009.1
MHQKRTAMFQDPQERPRKLPQLCTELQTTIHDIILECVYCKQQLLRREVYDFAFRDLCIVYRDGNPYAVCDKCLKFYS
KISEYRHYCYSVYGTTLEQQYNKPLCDLLIRCINCQKPLCPEEKQRHLDKKQRFHNIRGRWTGRCMSCCRSSRTRRET
QL
SEQ ID NO: 22 E7, partial [Human papillomavirus] GenBank: QIQ28214.1
SQPLKQHYQIVTCCCGCDSNVRLVVQCTETDIREVQQLLLGTLNIVCPICA
SEQ ID NO: 23 nucleo protein N [Rabies lyssavirus] GenBank: AFR56624.1
MDADKIVFKVNNQVVSLKPEIIVDQYEYKYPAIKDLKKPCITLGKAPDLNKAYKSVLSCMSAAKLDPDDVCSYLAAAM
QFFEGTCPEDWTSYGILIARKGDKITPGSLVEIKRTDVEGNWALTGGMELTRDPTVPEHASLVGLLLSLYRLSKISGQ
NTGNYKTNIADRIEQIFETAPFVKIVEHHTLMTTHKMCANWSTIPNFRFLAGTYDMFFSRIEHLYSAIRVGTVVTAYE
DCSGLVSFTGFIKQINLTAREAILYFFHKNFEEEIRRMFEPGQETAVPHSYFIHFRSLGLSGKSPYSSNAVGHVENLI
HFVGCYMGQVRSLNATVIAACAPHEMSVLGGYLGEEFFGKGTFERRFFRDEKELQEYEAAELTKTDVALADDGTVNSD
DEDYFSGETRSPEAVYTRIIMNGGRLKRSHIRRYVSVSSNHQARPNSFAEFLNKTYSSDS
SEQ ID NO: 24 L protein, partial [Rabies lyssavirus] NCBI Ref: ABH03115.1
MIDPGEVYDDP
SEQ ID NO: 25 transmembrane glycoprotein G [Rabies lyssavirus] NCBI Ref: AFR56627.1
MVPQALLFVPLLVFPLCFGKFPIYTIPDKLGPWSPIDIHHLSCPNNLVVEDEGCTNLSGFSYMELKVGYISAIKINGF
TCTGVVTEAETYTNFVGYVTTTFKRKHFRPTPDACRAAYNWKMAGDPRYEESLHNPYPDYHWLRTVKTTKESLVIISP
SVADLDPYDRSLHSRVFPGGNCSGVAVSSTYCSTNHDYTIWMPENPRLGMSCDIFTNSRGKRASKGSETCGFVDERGL
YKSLKGACKLKLCGVLGLRLMDGTWVAMQTSNETKWCPPGQLVNLHDFRSDEIEHLVVEELVKKREECLDALESIMTT
KSVSFRRLSHLRKLVPGFGKAYTIFNKTLMEADAHYKSVRTWNEIIPSKGCLRVGGRCHPHVNGVFFNGIILGPDGNV
LIPEMQSSLLQQHMELLVSSVIPLMHPMADPSTVFKNGDEAEDFVEVHLPDVHERISGVDLGLPNWGKYVLLSAGALT
ALMLIIFLMTCWRRVNRSEPTQHNLRGTGREVSVTPQSGKIISSWESYKSGGETGL
SEQ ID NO: 26 phosphoprotein M1 [Rabies lyssavirus] NCBI Ref: AFR56625.1
MSKIFVNPSAIRAGLADLEMAEETVDLINRNIEDNQAHLQGEPIEVDNLPEDMGRLHLDDGKSPNPGEMAKVGEGKYR
EDFQMDEGEDPSLLFQSYLDNVGVQIVRQIRSGERFFKIWSQTVEEIISYVAVNFPNPPGKSSEDKSTQTTGRELKKE
TTPTPSQRESQSSKARMAAQTASGPPALEWSATNEEDDLSVEAEIAHQIAESFSKKYKFPSRSSGILLYNFEQLKMNL
DDIVKEAKNVPGVTRLARDGSKLPLRCVLGWVALANSKKFQLLVESNKLSKIMQDDLNRYTSC
SEQ ID NO: 27 M2 protein [Rabies lyssavirus] NCBI Ref: AFR56626.1
MNFLRKIVKNCRDEDTQKPSPVSAPLDDDDLWLPPPEYVPLKELTSKKNRRNFCINGGVKVCSPNGYSFGILRHILRS
FDEIYSGNHRMIGLVKVVIGLALSGAPVPEGMNWVYKLRRTLIFQWADSRGPLEGEELEYSQEITWDDDTEFVGLQIR
VSAKQCHIRGRIWCINMNSRAGQLWSDMSLQTQRSEEDKDSSLLLE
SEQ ID NO: 28 envelope glycoprotein B [Human alphaherpesvirus 1] NCBI Ref: YP_009137102.1
MRQGAPARGRRWFVVWALLGLTLGVLVASAAPSSPGTPGVAAATQAANGGPATPAPPAPGAPPTGDPKPKKNKKPKPP
KPPRPAGDNATVAAGHATLREHLRDIKAENTDANFYVCPPPTGATVVQFEQPRRCPTRPEGQNYTEGIAVVFKENIAP
YKFKATMYYKDVTVSQVWFGHRYSQFMGIFEDRAPVPFEEVIDKINAKGVCRSTAKYVRNNLETTAFHRDDHETDMEL
KPANAATRTSRGWHTTDLKYNPSRVEAFHRYGTTVNCIVEEVDARSVYPYDEFVLATGDFVYMSPFYGYREGSHTEHT
SYAADRFKQVDGFYARDLTTKARATAPTTRNLLTTPKFTVAWDWVPKRPSVCTMTKWQEVDEMLRSEYGGSFRFSSDA
ISTTFTTNLTEYPLSRVDLGDCIGKDARDAMDRIFARRYNATHIKVGQPQYYLANGGFLIAYQPLLSNTLAELYVREH
LREQSRKPPNPTPPPPGASANASVERIKTTSSIEFARLQFTYNHIQRHVNDMLGRVAIAWCELQNHELTLWNEARKLN
PNAIASATVGRRVSARMLGDVMAVSTCVPVAADNVIVQNSMRISSRPGACYSRPLVSFRYEDQGPLVEGQLGENNELR
LTRDAIEPCTVGHRRYFTFGGGYVYFEEYAYSHQLSRADITTVSTFIDLNITMLEDHEFVPLEVYTRHEIKDSGLLDY
TEVQRRNQLHDLRFADIDTVIHADANAAMFAGLGAFFEGMGDLGRAVGKVVMGIVGGVVSAVSGVSSFMSNPFGALAV
GLLVLAGLAAAFFAFRYVMRLQSNPMKALYPLTTKELKNPTNPDASGEGEEGGDFDEAKLAEAREMIRYMALVSAMER
TEHKAKKKGTSALLSAKVTDMVMRKRRNTNYTQVPNKDGDADEDDL
SEQ ID NO: 29 glycoprotein G [Human alphaherpesvirus 2] NCBI Ref: ABU45441.1
MHAIAPRLLLLFVLSGLPGTRGGSGVPGPINPPNNDVVFPGGSPVAQYCYAYPRLDDPGPLGSADAGRQDLPRRVVRH
EPLGRSFLTGGLVLLAPPVRGFGAPNATYAARVTYYRLTRACRQPILLRQYGGCRGGEPPSPKTCGSYTYTYQGGGPP
TRYALVNASLLVPIWDRAAETFEYQIELGGELHVGLLWVEVGGEGPGPTAPPQAARAEGGPCVPPVPAGRPWRSVPPV
WYSAPNPGFRGLRFRERCLPPQTPAAPSDLPRVAFAPQSLLVGITGRTFIRMARPTEDVGVLPPHWAPGALDDGPYAP
FPPRPRFRRALRTDPEGVDPDVRAPLTGRRLMALTEDASSDSPTSAPEKTPLPVSATAMAPSVDPSAEPTAPATTTPP
DEMATQAATVAVTPEETAVASPPATASVESSPLPAAAATPGAGHTNTSSAPAAKTPPTTPAPTTPPPTSTHATPRPTT
PGPQTTPPGPATPGPVGASAAPTADSPLTASPPATAPGPSAANVSVAATTATPGTRGTARTPPTDPKTHPHGPADAPP
GSPAPPPPEHRGGPEEFEGAGDGEPPDDDDSATGLAFRTPNPNKPPPARPGPIRPTLPPGILGPLAPNTPRPPAQAPA
KDMPSGPTPQHIPLFWFLTASPALDILFIISTTIHTAAFVCLVALAAQLWRGRAGRRRYAHPSVRYVCLPPERD
SEQ ID NO: 30 Malate synthase [Mycobacterium tuberculosis] NCBI Ref: QJF21868.1
MTDRVSVGNLRIARVLYDFVNNEALPGTDIDPDSFWAGVDKVVADLTPQNQALLNARDELQAQIDKWHRRRVIEPIDM
DAYRQFLTEIGYLLPEPDDFTITTSGVDAEITTTAGPQLVVPVLNARFALNAANARWGSLYDALYGTDVIPETDGAEK
GPTYNKVRGDKVIAYARKFLDDSVPLSSGSFGDATGFTVQDGQLVVALPDKSTGLANPGQFAGYTGAAESPTSVLLIN
HGLHIEILIDPESQVGTTDRAGVKDVILESAITTIMDFEDSVAAVDAADKVLGYRNWLGLNKGDLAAAVDKDGTAFLR
VLNRDRNYTAPGGGQFTLPGRSLMFVRNVGHLMTNDAIVDTDGSEVFEGIMDALFTGLIAIHGLKASDVNGPLINSRT
GSIYIVKPKMHGPAEVAFTCELFSRVEDVLGLPQNTMKIGIMDEERRTTVNLKACIKAAADRVVFINTGFLDRTGDEI
HTSMEAGPMVRKGTMKSQPWILAYEDHNVDAGLAAGFSGRAQVGKGMWTMTELMADMVETKIAQPRAGASTAWVPSPT
AATLHALHYHQVDVAAVQQGLAGKRRATIEQLLTIPLAKELAWAPDEIREEVDNNCQSILGYVVRWVDQGVGCSKVPD
IHDVALMEDRATLRISSQLLANWLRHGVITSADVRASLERMAPLVDRQNAGDVAYRPMAPNFDDSIAFLAAQELILSG
AQQPNGYTEPILHRRRREFKARAAEKPAPSDRAGDDAAR
SEQ ID NO: 31 MPT51 [Mycobacterium tuberculosis H37Rv] NCBI Ref: CAA05211.1
MKGRSALLRALWIAALSFGLGGVAVAAEPTAKAAPYENLMVPSPSMGRDIPVAFLAGGPHAVYLLDAFNAGPDVSNWV
TAGNAMNTLAGKGISVVAPAGGAYSMYTNWEQDGSKQWDTFLSAELPDWLAANRGLAPGGHAAVGAAQGGYGAMALAA
FHPDRFGFAGSMSGFLYPSNTTTNGAIAAGMQQFGGVDTNGMWGAPQLGRWKWHDPWVHASLLAQNNTRVWVWSPTNP
GASDPAAMIGQTAEAMGNSRMFYNQYRSVGGHNGHFDFPASGDNGWGSWAPQLGAMSGDIVGAIR
SEQ ID NO: 32 Zika Virus E Antigen Amino Acid Sequence
polyprotein, partial [Zika virus] GenBank: ART29827.1
MKNPKKKSGGFRIVNMLKRGVARVSPFGGLKRLPAGLLLGHGPIRMVLAILAFLRFTAIKPSLGLINRWGSVGKKEAM
EIIKKFKKDLAAMLRIINARKEKKRRGADTSVGIVGLLLTTAMAAEVTRRGSAYYMYLDRNDAGEAISFPTTLGMNKC
YIQIMDLGHMCDATMSYECPMLDEGVEPDDVDCWCNTTSTWVVYGTCHHKKGEARRSRRAVTLPSHSTRKLQTRSQTW
LESREYTKHLIRVENWIFRNPGFALAAAAIAWLLGSSTSQKVIYLVMILLIAPAYSIRCIGVSNRDFVEGMSGGTWVD
VVLEHGGCVTVMAQDKPTVDIELVTTTVSNMAEVRSYCYEASISDMASDSRCPTQGEAYLDKQSDTQYVCKRTLVDRG
WGNGCGLFGKGSLVTCAKFACSKKMTGKSIQPENLEYRIMLSVHGSQHSGMIVNDTGHETDENRAKVEITPNSPRAEA
TLGGFGSLGLDCEPRTGLDFSDLYYLTMNNKHWLVHKEWFHDIPLPWHAGADTGTPHWNNKEALVEFKDAHAKRQTVV
VLGSQEGAVHTALAGALEAEMDGAKGRLSSGHLKCRLKMDKLRLKGVSYSLCTAAFTFTKIPAETLHGTVTVEVQYAG
TDGPCKVPAQMAVDMQTLTPVGRLITANPVITESTENSKMMLELDPPFGDSYIVIGVGEKKITHHWHRSGSTIGKAFE
ATVRGAKRMAVLGDTAWDFGSVGGALNSLGKGIHQIFGAAFKSLFGGMSWFSQILIGTLLMWLGLNTKNGSISLMCLA
LGGVLIFLSTAVSADVGCSVDFSKKETRCGTGVFVYNDVEAWRDRYKYHPDSPRRLAAAVKQAWEDGICGISSVSRME
NIMWRSVEGELNAILEENGVQLTVVVGSVKNPMWRAPQRLPVPVNELPHGWKAWGKSYFVRAAKTNNSFVVDGDTLKE
CPLKHRAWNSFLVEDHGFGVFHTSVWLKVREDYSLECDPAVIGTAVKGKEAVHSDLGYWIESEKNDTWRLKRAHLIEM
KTCEWPKSHTLWTDGIEESDLIIPTSLAGPLSHHNTREGYRTQMKGPWHSEELEIRFEECPGTKVHVEETCGTRGPSL
RSTTASGRVIEEWCCRECTMPPLSFRAKDGCWYGMEIRPRKEPESNLVRSMVTAGSTDHMDHFSLGVLVILLMVQEGL
KKRMTTKIIISTSMAVLVAMILGGFSMSDLAKLAILMGATFAEMNTGGDVAHLALIAAFKVRPALLVSFIFRANWTPR
ESMLLALASCLLQTAISALEGDLMVLINGFALAWLAIRAMVVPRTDNITLAILAALTPLARGTLLVAWRAGLATCGGF
MLLSLKGKGSVKKNLPFVMALGLTAVRLVDPINVVGLLLLTRSGKRSWPPSEVLTAVGLICALAGGFAKADIEMAGPM
AAVGLLIVSYVVSGKSVDMYIERAGDITWEKDAEVTGNSPRLDVALDESGDFSLVEDDGPPMREIILKVVLMTICGMN
PIAIPFAAGAWYVYVKTWKRSGALWDVPASKEVKKGETTDGVYRVMTRRLLGSTQVGVGIMQEGVFHTMWHVTKGSAL
RSGEGRLDPYWGDVKQDLVSYCGPWKLDAAWDGHSEVQLLAVPPGERARNIQTLPGIFKTKDGDIGAVALDYPAGTSG
SPILDKCGRVIGLYGNGVVIKNGSYVSAITQGRREEETPVECFEPSMLKKKQLTVLDLHPGAGKTRRVLPEIVREAIK
TRLRTVILAPTRVVAAEMEEALRGLPVRYMTTAVNVTHSGTEIVDLMCHATFTSRLLQPIRVPNYNLYIMDEAHFTDP
SSIAARGYISTRVEMGEAAAIFMTATPPGTRDAFPDSNSPIMDTEVEVPERAWSSGFDWVTDHSGKTVWFVPSVRNGN
EIAACLTKAGKRVIQLSRKTFETEFQKTKHQEWDFVVTTDISEMGANFKADRVIDSRRCLKPVILDGERVILAGPMPV
THASAAQRRGRIGRNPNKPGDEYLYGGGCAETDEDHAHWLEARMLLDNIYLQDGLIASLYRPEADKVAAIEGEFKLRT
EQRKTFVELMKRGDLPVWLAYQVASAGITYTDRRWCFDGTTNNTILEDSVPAEVWTRHGEKRVLKPRWMDARVCSDHA
ALKSFKEFAAGKRGAAFGVMEALGTLPGHMTERFQEAIDNLAVLMRAEIGSRPYKAAAAQLPETLETIMLLGLLGTVS
LGIFFVLMRNKGIGKMGFGMVTLGASAWLMWLSEIEPARIACVLIVVFLLLVVLIPEPEKQRSPQDNQMAIIIMVAVG
LLGLITANELGWLERTKSDLSHLMGRREEGATIGFSMDIDLRPASAWAIYAALTTFITPAVQHAVTTSYNNYSLMAMA
TQAGVLFGMGKGMPFYAWDFGVPLLMIGCYSQLTPLTLIVAIILLVAHYMYLIPGLQAAAARAAQKRTAAGIMKNPVV
DGIVVTDIDTMTIDPQVEKKMGQVLLIAVAVSSAILSRTAWGWGEAGALITAATSTLWEGSPNKYWNSSTATSLCNIF
RGSYLAGASLIYTVTRNAGLVKRRGGGTGETLGEKWKARLNQMSALEFYSYKKSGITEVCREEARRALKDGVATGGHA
VSRGSAKLRWLVERGYLQPYGKVIDLGCGRGGWSYYAATIRKVQEVKGYTKGGPGHEEPVLVQSYGWNIVRLKSGVDV
FHMAAEPCDTLLCDIGESSSSPEVEEARTLRVLSMVGDWLEKRPGAFCIKVLCPYTSTMMETLERLQRRYGGGLVRVP
LSRNSTHEMYWVSGAKSNTIKSVSTTSQLLLGRMDGPRRPVKYEEDVNLGSGTRAVVSCAEAPNMKIIGNRIERIRSE
HAETWFFDENHPYRTWAYHGSYEAPTQGSASSLINGVVRLLSKPWDVVTGVTGIAMTDTTPYGQQRVFKEKVDTRVPD
PQEGTRQVMSMVSSWLWKELGKHKRPRVCTKEEFINKVRSNAALGAIFEEEKEWKTAVEAVNDPRFWALVDKEREHHL
RGECQSCVYNMMGKREKKQGEFGKAKGSRAIWYMWLGARFLEFEALGFLNEDHWMGRENSGGGVEGLGLQRLGYVLEE
MSCIPGGRMYADDTAGWDTRISRFDLENEALITNQMEKGHRALALAIIKYTYQNKVVKVLRPAEKGKTVMDIISRQDQ
RGSGQVVTYALNTFTNLVVQLIRNMEAEEVLEMQDLWLLRRSEKVTNWLQSNGWDRLKRMA
SEQ ID NO: 33 major outer membrane protein precursor [Chlamydia trachomatis] NCBI Ref:
AAA23156.1
MKKLLKSVLVFAALSSASSLQALPVGNPAEPSLMIDGILWEGFGGDPCDPCTTWCDAISMRVGYYGDFVFDRVLKTDV
NKEFQMGAAPTTSDVAGLQNDPTINVARPNPAYGKHMQDAEMFTNAAYMALNIWDRFDVFCTLGATTGYLKGNSASFN
LVGLFGTKTQSSSFNTAKLIPNTALNEAVVELYINTTFAWSVGARAALWECGCATLGASFQYAQSKPKVEELNVLCNA
SEFTINKPKGYVGAEFPLNITAGTEAATGTKDASIDYHEWQASLALSYRLNMFTPYIGVKWSRVSFDADTIRIAQPKL
AEAILDVTTLNRTTAGKGSVVSAGTDNELADTMQIVSLQLNKMKSRKSCGIAVGTTIVDADKYAVTVEARLIDERAAH
VNAQFRF
SEQ ID NO: 34 VEEV RNA Sequence
AUAGGCGGCGCAUGAGAGAAGCCCAGACCAAUUACCUACCCAAAAUGGAGAAAGUUCACGUUGACAUCGAGGAAGACA
GCCCAUUCCUCAGAGCUUUGCAGCGGAGCUUCCCGCAGUUUGAGGUAGAAGCCAAGCAGGUCACUGAUAAUGACCAUG
CUAAUGCCAGAGCGUUUUCGCAUCUGGCUUCAAAACUGAUCGAAACGGAGGUGGACCCAUCCGACACGAUCCUUGACA
UUGGAAGUGCGCCCGCCCGCAGAAUGUAUUCUAAGCACAAGUAUCAUUGUAUCUGUCCGAUGAGAUGUGCGGAAGAUC
CGGACAGAUUGUAUAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGACGACGAGUCGUGUC
GCUACGAAGGGCAAGUCGCUGUUUACCAGGAUGUAUACGCGGUUGACGGACCGACAAGUCUCUAUCACCAAGCCAAUA
AGGGAGUUAGAGUCGCCUACUGGAUAGGCUUUGACACCACCCCUUUUAUGUUUAAGAACUUGGCUGGAGCAUAUCCAU
CAUACUCUACCAACUGGGCCGACGAAACCGUGUUAACGGCUCGUAACAUAGGCCUAUGCAGCUCUGACGUUAUGGAGC
GGUCACGUAGAGGGAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGUGGCAAGCAAAAUU
ACACAUGUCGGUGUGAGACUAUAGUUAGUUGCGACGGGUACGUCGUUAAAAGAAUAGCUAUCAGUCCAGGCCUGUAUG
GGAAGCCUUCAGGCUAUGCUGCUACGAUGCACCGCGAGGGAUUCUUGUGCUGCAAAGUGACAGACACAUUGAACGGGG
AGAGGGUCUCUUUUCCCGUGUGCACGUAUGUGCCAGCUACAUUGUGUGACCAAAUGACUGGCAUACUGGCAACAGAUG
UCAGUGCGGACGACGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAAUAUAAGGAAGAUC
AAGAAGAUGAAAGGCCACUAGGACUACGAGAUAGACAGUUAGUCAUGGGGUGUUGUUGGGCUUUUAGAAGGCACAAGA
UAACAUCUAUUUAUAAGCGCCCGGAUACCCAAACCAUCAUCAAAGUGAACAGCGAUUUCCACUCAUUCGUGCUGCCCA
GGAUAGGCAGUAACACAUUGGAGAUCGGGCUGAGAACAAGAAUCAGGAAAAUGUUAGAGGAGCACAAGGAGCCGUCAC
CUCUCAUUACCGCCGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGACUUGAUGUUACAAG
AGGCUGGGGCCGGCUCAGUGGAGACACCUCGUGGCUUGAUAAAGGUUACCAGCUACGAUGGCGAGGACAAGAUCGGCU
CUUACGCUGUGCUUUCUCCGCAGGCUGUACUCAAGAGUGAAAAAUUAUCUUGCAUCCACCCUCUCGCUGAACAAGUCA
UAGUGAUAACACACUCUGGCCGAAAAGGGCGUUAUGCCGUGGAACCAUACCAUGGUAAAGUAGUGGUGCCAGAGGGAC
AUGCAAUACCCGUCCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACUGUCAAGCCCAGCG
AGCACGACGGCGAAUACCUGUACGACAUCGACAGGAAACAGUGCGUCAAGAAAGAACUAGUCACUGGGCUAGGGCUCA
CAGGCGAGCUGGUGGAUCCUCCCUUCCAUGAAUUCGCCUACGAGAGUCUGAGAACACGACCAGCCGCUCCUUACCAAG
UACCAACCAUAGGGGUGUAUGGCGUGCCAGGAUCAGGCAAGUCUGGCAUCAUUAAAAGCGCAGUCACCAAAAAAGAUC
UAGUGGUGAGCGCCAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGACGAAGCUUUUGCUU
GUCAUGCAGGUACUCUCAGAGCGCUCAUAGCCAUUAUAAGACCUAAAAAGGCAGUGCUCUGCGGGGAUCCCAAACAGU
GCGGUUUUUUUAACAUGAUGUGCCUGAAAGUGCAUUUUAACCACGAGAUUUGCACACAAGUCUUCCACAAAAGCAUCU
CUCGCCGUUGCACUAAAUCUGUGACUUCGGUCGUCUCAACCUUGUUUUACGACAAAAAAAUGAGAACGACGAAUCCGA
AAGAGACUAAGAUUGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAAGGGCUGACCCGUA
AAGGUGUGUAUGCCGUUCGGUACAAGGUGAAUGAAAAUCCUCUGUACGCACCCACCUCAGAACAUGUGAACGUCCUAC
UGACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACCGG
ACCCUACCGACGUCUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCAGAGAUAGUAUUGA
ACCAACUAUGCGUGAGGUUCUUUGGACUCGAUCUGGACUCCGGUCUAUUUUCUGCACCCACUGUUCCGUUAUCCAUUA
GGAAUAAUCACUGGGAUAACUCCCCGUCGCCUAACAUGUACGGGCUGAAUAAAGAAGUGGUCCGUCAGCUCUCUCGCA
GGUACCCACAACUGCCUCGGGCAGUUGCCACUGGAAGAGUCUAUGACAUGAACACUGGUACACUGCGCAAUUAUGAUC
CGCGCAUAAACCUAGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCCGUCCCAGGCAAAA
UGGUUGACUGGUUGUCAGACCGGCCUGAGGCUACCUUCAGAGCUCGGCUGGAUUUAGGCAUCCCAGGUGAUGUGCCCA
AAUAUGACAUAAUAUUUGUUAAUGUGAGGACCCCAUAUAAAUACCAUCACUAUCAGCAGUGUGAAGACCAUGCCAUUA
AGCUUAGCAUGUUGACCAAGAAAGCUUGUCUGCAUCUGAAUCCCGGCGGAACCUGUGUCAGCAUAGGUUAUGGUUACG
CUGACAGGGCCAGCGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAUCCUUACAAGCUUU
CAUCAACCUUGACCAACAUUUAUACAGGUUCCAGACUCCACGAAGCCGGAUGUGCACCCUCAUAUCAUGUGGUGCGAG
GGGAUAUUGCCACGGCCACCGAAGGAGUGAUUAUAAAUGCUGCUAACAGCAAAGGACAACCUGGCGGAGGGGUGUGCG
GAGCGCUGUAUAAGAAAUUCCCGGAAAGCUUCGAUUUACAGCCGAUCGAAGUAGGAAAAGCGCGACUGGUCAAAGGUG
CAGCUAAACAUAUCAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUGUUGUCCACCGGCA
UCUUUUCCGGGAACAAAGAUCGACUAACCCAAUCAUUGAACCAUUUGCUGACAGCUUUAGACACCACUGAUGCAGAUG
UAGCCAUAUACUGCAGGGACAAGAAAUGGGAAAUGACUCUCAAGGAAGCAGUGGCUAGGAGAGAAGCAGUGGAGGAGA
UAUGCAUAUCCGACGACUCUUCAGUGACAGAACCUGAUGCAGAGCUGGUGAGGGUGCAUCCGAAGAGUUCUUUGGCUG
GAAGGAAGGGCUACAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAUAUCCUCGGAGAAA
GCAUGAGCAGUAUUAGGUCGAAAUGCCCCGUCGAAGAGUCGGAAGCCUCCACACCACCUAGCACGCUGCCUUGCUUGU
GCAUCCAUGCCAUGACUCCAGAAAGAGUACAGCGCCUAAAAGCCUCACGUCCAGAACAAAUUACUGUGUGCUCAUCCU
UUCCAUUGCCGAAGUAUAGAAUCACUGGUGUGCAGAAGAUCCAAUGCUCCCAGCCUAUAUUGUUCUCACCGAAAGUGC
CUGCGUAUAUUCAUCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACGCCUGAGCCGAUCA
UCAUCGAAGAGGAAGAAGAGGAUAGCAUAAGUUUGCUGUCAGAUGGCCCGACCCACCAGGUGCUGCAAGUCGAGGCAG
ACAUUCACGGGCCGCCCUCUGUAUCUAGCUCAUCCUGGUCCAUUCCUCAUGCAUCCGACUUUGAUGUGGACAGUUUAU
CCAUACUUGACACCCUGGAGGGAGCUAGCGUGACCAGCGGGGCAACGUCAGCCGAGACUAACUCUUACUUCGCAAAGA
GUAUGGAGUUUCUGGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGCGUGAAUAGGGUGA
UCACUAGAGAGGAGCUCGAGGCGCUUACCCCGUCACGCACUCCUAGCAGGUCGGUCUCGAGAACCAGCCUGGUCUCCA
ACCCGCCAGGCGUAAAUAGGGUGAUUACAAGAGAGGAGUUUGAGGCGUUCGUAGCACAACAACAAUGACGGUUUGAUG
CGGGUGCAUACAUCUUUUCCUCCGACACCGGUCAAGGGCAUUUACAACAAAAAUCAGUAAGGCAAACGGUGCUAUCCG
AAGUGGUGUUGGAGAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCAUC
CUGUUCCUUUGUAUUCAUCUAGUGUGAACCGUGCCUUUUCAAGCCCCAAGGUCGCAGUGGAAGCCUGUAACGCCAUGU
UGAAAGAGAACUUUCCGACUGUGGCUUCUUACUGUAUUAUUCCAGAGUACGAUGCCUAUUUGGACAUGGUUGACGGAG
CUUCAUGCUGCUUAGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCCACAAAAAGAAAUU
GCAAUGUCACGCAAAUGAGAGAAUUGCCCGUAUUGGAUUCGGCGGCCUUUAAUGUGGAAUGCUUCAAGAAAUAUGCGU
GUAAUAAUGAAUAUUGGGAAACGUUUAAAGAAAACCCCAUCAGGCUUACUGAAGAAAACGUGGUAAAUUACAUUACCA
AAUUAAAAGGACCAAAAGCUGCUGCUCUUUUUGCGAAGACACAUAAUUUGAAUAUGUUGCAGGACAUACCAAUGGACA
GGUUUGUAAUGGACUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGGAGAUUAAAUGCGG
UCCUGCUUCCGAACAUUCAUACACUGUUUGAUAUGUCGGCUGAAGACUUUGACGCUAUUAUAGCCGAGCACUUCCAGC
CUGGGGAUUGUGUUCUGGAAACUGACAUCGCGUCGUUUGAUAAAAGUGAGGACGACGCCAUGGCUCUGACCGCGUUAA
UGAUUCUGGAAGACUUAGGUGUGGACGCAGAGCUGUUGACGCUGAUUGAGGCGGCUUUCGGCGAAAUUUCAUCAAUAC
AUUUGCCCACUAAAACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCAUUCAUUGGAGAUG
ACAAUAUCGUGAAAGGAGUCAAAUCGGACAAAUUAAUGGCAGACAGGUGCGCCACCUGGUUGAAUAUGGAAGUCAAGA
UUAUAGAUGCUGUGGUGGGCGAGAAAGCGCCUUAUUUCUGUGGAGGGUUUAUUUUGUGUGACUCCGUGACCGGCACAG
CGUGCCGUGUGGCAGACCCCCUAAAAAGGCUGUUUAAGCUUGGCAAACCUCUGGCAGCAGACGAUGAACAUGAUGAUG
ACAGGAGAAGGGCAUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGUGUUAAAUCAUUCA
GCUACCUGAGAGGGGCCCCUAUAACUCUCUACGGCUAACCUGAAUGGACUACGACAUAGUCUAGUCCGCCAAG
SEQ ID NO: 35 VEEV RNA polymerase Amino Acid Sequence (NCBI Accession: AXP98866.1)
RELPVLDSAAFNVECFKKYACNNEYWETFKENPIRLTEENVVNYITKLKGP
SEQ ID NO: 36 VEEV RNA polymerase Amino Acid Sequence (NCBI Accession: AXP98867.1)
TQMRELPVLDSAAFNVECFKKYACNNEYWETFKENPIRLTE
SEQ ID NO: 37 Polyprotein Amino Acid Sequence [Venezuelan equine encephalitis virus]
(GenBank: ALE15116.1)
MKAITARRILQGLGHYLKAEGKVECYRTLHPVPLYSSSVNRAFSSPKVAVEACNAMLKENFPTVASYCIIPEYDAYLD
MIDGASCCLDTASFCPAKLRSFPKKHSYLEPTIRSAVPSAIQNTLQNVLAAATKRNCNVTQMRELPVLDSAAFNVECF
KKYACNNEYWKTFKENPIRLTEENVINYITKLKGPKAAALYAKTHNLNMLQDIPMDRFVMDLKRDVKVTPGTKHTEER
PKVQVIQAADPLATAYLCGIHRELVRRLNAVLLPNIHTLFDMSAEDFDAIIAEHFQPGDCVLETDIASFDKSEDDAMA
LTAMMILEDLGVDAELLTLIEAAFGEISSIHLPTKTKFKFGAMMKSGMFLTLFVNTVINIVIASRVLRERLTGSPCAA
FIGDDNIVKGVKSDKLMADRCATWLNMEVKIIDAVVGEKAPYFCGGFILCDSVTGTACRVADPLKRLFKLGKPLAADD
EHDDDRRRALHEESTRWNRVGILPELCKAVESRYETVGTSVIVMAMATLASSVKSFSYLRGAPITLYG

The following sequences (SEQ ID NOS: 38-43) are formatted to signify vector backbone and antigen open reading frames as follows: lower case letters signify the vector backbone sequence; and UPPER CASE letters signify the VZV antigen open reading frame.

SEQ ID NO: 38
VEEV-kozak-VZV-FL-gE
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacguaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgcCGCCACCAU
GUUCUAUGAAGCUUUGAAGGCCGAACUUGUAUACACCCGAGCCGUGCAUGGUUUCCGCCCGAGAGCGAACUGCGUCGU
ACUGUCAGAUUACAUCCCCAGAGUCGCGUGCAACAUGGGUACGGUUAAUAAGCCCGUGGUGGGGGUGCUGAUGGGUUU
UGGGAUAAUCACCGGAACUCUGCGCAUCACAAAUCCUGUAAGAGCGUCAGUGCUGAGGUAUGACGAUUUCCACAUAGA
CGAGGACAAGCUGGAUACCAAUUCCGUUUACGAACCAUAUUAUCACUCCGAUCACGCCGAAUCCAGCUGGGUAAAUCG
AGGAGAGUCCUCACGGAAGGCAUACGACCAUAACUCCCCAUAUAUAUGGCCUCGCAAUGAUUAUGACGGAUUCCUGGA
AAACGCCCAUGAGCACCACGGCGUUUAUAACCAGGGUCGCGGUAUCGAUUCUGGGGAAAGGUUGAUGCAACCAACUCA
GAUGUCCGCUCAGGAAGAUUUGGGGGAUGACACCGGAAUCCAUGUGAUACCUACACUCAAUGGUGAUGAUCGACACAA
AAUUGUAAACGUGGACCAGAGGCAAUACGGGGACGUUUUUAAGGGAGACCUGAAUCCAAAACCACAAGGACAACGACU
CAUCGAGGUUUCUGUAGAAGAAAACCAUCCGUUUACGCUCCGCGCCCCCAUACAGCGCAUAUAUGGCGUGCGCUAUAC
GGAGACCUGGUCUUUUCUCCCCAGUCUGACAUGCACUGGUGAUGCAGCUCCAGCAAUUCAGCACAUAUGUCUCAAGCA
UACCACCUGUUUCCAAGAUGUAGUAGUGGAUGUGGAUUGCGCCGAAAACACGAAAGAAGACCAGCUUGCCGAGAUUUC
AUACCGGUUUCAAGGAAAAAAAGAAGCCGACCAGCCGUGGAUAGUAGUGAAUACGUCCACGCUCUUCGAUGAACUGGA
AUUGGACCCUCCUGAAAUUGAACCCGGAGUCCUGAAAGUUUUGCGGACUGAGAAACAGUAUCUCGGUGUGUACAUCUG
GAAUAUGAGGGGCAGUGAUGGAACAAGCACAUACGCGACCUUUCUGGUGACAUGGAAAGGUGACGAAAAGACUAGAAA
CCCAACCCCAGCAGUGACCCCCCAACCUAGAGGUGCUGAGUUCCACAUGUGGAAUUACCACUCCCAUGUAUUCUCUGU
CGGAGACACCUUCAGCUUGGCGAUGCACCUCCAAUACAAGAUCCAUGAAGCCCCGUUCGACCUCUUGUUGGAGUGGCU
CUACGUACCAAUAGACCCUACCUGUCAACCAAUGAGGCUUUACUCUACAUGCCUUUACCACCCAAACGCACCACAAUG
UCUUUCUCAUAUGAAUAGCGGUUGUACAUUUACAUCUCCCCAUCUCGCGCAAAGAGUCGCGAGUACAGUUUACCAAAA
UUGUGAGCAUGCGGACAAUUACACAGCAUAUUGUCUUGGGAUCUCCCACAUGGAGCCCUCUUUUGGGCUUAUACUUCA
CGACGGGGGUACAACGCUUAAGUUCGUCGAUACGCCAGAAUCCCUCUCUGGCUUGUAUGUAUUCGUGGUUUAUUUUAA
CGGUCACGUUGAGGCUGUGGCCUAUACUGUUGUUAGCACUGUAGACCACUUUGUUAACGCCAUAGAAGAGAGGGGGUU
CCCGCCCACCGCUGGCCAGCCACCGGCCACAACGAAGCCUAAAGAGAUUACGCCGGUGAAUCCCGGCACAAGUCCCCU
UAUCCGGUACGCCGCAUGGACAGGAGGACUGGCGGCCGUCGUCCUCCUUUGUCUGGUUAUAUUCCUUAUAUGCACGGC
AAAGCGAAUGCGGGUUAAGGCUUACCGAGUGGAUAAAUCUCCAUAUAAUCAGAGCAUGUAUUACGCAGGGCUCCCUGU
GGACGACUUUGAGGAUAGCGAGUCCACGGAUACAGAAGAGGAAUUUGGAAACGCCAUCGGUGGGAGCCAUGGAGGGUC
AUCUUACACCGUUUACAUUGACAAAACUAGAUGAuaaccgcggugucaaaaaccgcguggacgugguuaacaucccug
cugggaggaucagccguaauuauuauaauuggcuuggugcuggcuacuauuguggccauguacgugcugaccaaccag
aaacauaauugaauacagcagcaauuggcaagcugcuuacauagaacucgcggcgauuggcaugccgccuuaaaauuu
uuauuuuauuuuuucuuuucuuuuccgaaucggauuuuguuuuuaauauuucaaaaaaaaaaaaaaaaaaaaaaaaaa
aaaaaaaaaaaaaaaaaaa
SEQ ID NO: 39
VEEV-kozak-FL-gI
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacguaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
acccuaccgacgucuuccagaauaaggcaaacguguguugggccaaggcuuuagugccggugcugaagaccgcuggca
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagcaguuuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgcCGCCACCAU
GUUCCUUAUUCAAUGCCUCAUAUCCGCAGUCAUCUUCUAUAUUCAAGUUACUAAUGCACUUAUCUUCAAGGGCGAUCA
UGUCAGCCUGCAAGUGAAUUCAAGUCUUACGUCCAUUUUGAUUCCAAUGCAGAAUGAUAACUAUACCGAAAUUAAGGG
ACAGCUGGUUUUUAUUGGAGAACAACUGCCGACCGGUACAAACUACAGCGGGACCCUCGAGCUGCUUUACGCAGACAC
UGUGGCAUUCUGCUUUCGGUCAGUACAAGUUAUCAGAUACGACGGAUGCCCACGCAUUAGGACAUCUGCCUUCAUUUC
UUGCCGAUACAAGCAUAGUUGGCAUUACGGAAACUCUACCGAUAGAAUUUCAACUGAACCAGAUGCCGGUGUGAUGCU
CAAGAUAACCAAACCUGGGAUCAACGACGCAGGGGUGUAUGUCCUCUUGGUGAGAUUGGACCAUUCAAGGAGUACGGA
CGGGUUUAUACUGGGCGUGAACGUCUAUACCGCAGGAAGUCAUCAUAACAUUCACGGUGUCAUUUAUACCAGCCCCAG
UCUCCAGAAUGGGUACAGCACUCGAGCCCUGUUCCAGCAAGCAAGAUUGUGUGACCUUCCAGCCACUCCUAAGGGAUC
AGGCACAAGUCUUUUUCAACAUAUGUUGGAUCUCAGAGCAGGGAAAAGUCUUGAGGACAACCCGUGGCUCCAUGAAGA
CGUGGUUACUACUGAAACAAAGUCAGUGGUCAAGGAGGGAAUCGAGAACCAUGUGUACCCAACUGACAUGAGCACGCU
GCCUGAAAAAUCACUGAACGACCCACCAGAGAAUCUGCUGAUAAUAAUACCUAUUGUAGCGAGUGUUAUGAUUUUGAC
CGCAAUGGUCAUAGUUAUUGUAAUAAGCGUGAAAAGGAGACGAAUCAAAAAACAUCCGAUAUACAGGCCGAAUACGAA
GACAAGAAGAGGGAUUCAGAACGCGACUCCGGAGAGUGAUGUAAUGCUCGAAGCAGCCAUCGCUCAACUUGCCACCAU
UCGCGAAGAAAGCCCUCCGCAUUCCGUCGUAAAUCCUUUUGUCAAGUGAuaaccgcggugucaaaaaccgcguggacg
ugguuaacaucccugcugggaggaucagccguaauuauuauaauuggcuuggugcuggcuacuauuguggccauguac
gugcugaccaaccagaaacauaauugaauacagcagcaauuggcaagcugcuuacauagaacucgcggcgauuggcau
gccgccuuaaaauuuuuauuuuauuuuuucuuuucuuuuccgaaucggauuuuguuuuuaauauuucaaaaaaaaaaa
aaaaaaaaaaaaaaaaaaaaaaaaaaaaaaaaaa
SEQ ID NO: 40
VZV-N term trunc gE
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacguaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgcCGCCACCAU
GGGUACGGUUAAUAAGCCCGUGGUGGGGGUGCUGAUGGGUUUUGGGAUAAUCACCGGAACUCUGCGCAUCACAAAUCC
UGUAAGAGCGUCAGUGCUGAGGUAUGACGAUUUCCACAUAGACGAGGACAAGCUGGAUACCAAUUCCGUUUACGAACC
AUAUUAUCACUCCGAUCACGCCGAAUCCAGCUGGGUAAAUCGAGGAGAGUCCUCACGGAAGGCAUACGACCAUAACUC
CCCAUAUAUAUGGCCUCGCAAUGAUUAUGACGGAUUCCUGGAAAACGCCCAUGAGCACCACGGCGUUUAUAACCAGGG
UCGCGGUAUCGAUUCUGGGGAAAGGUUGAUGCAACCAACUCAGAUGUCCGCUCAGGAAGAUUUGGGGGAUGACACCGG
AAUCCAUGUGAUACCUACACUCAAUGGUGAUGAUCGACACAAAAUUGUAAACGUGGACCAGAGGCAAUACGGGGACGU
UUUUAAGGGAGACCUGAAUCCAAAACCACAAGGACAACGACUCAUCGAGGUUUCUGUAGAAGAAAACCAUCCGUUUAC
GCUCCGCGCCCCCAUACAGCGCAUAUAUGGCGUGCGCUAUACGGAGACCUGGUCUUUUCUCCCCAGUCUGACAUGCAC
UGGUGAUGCAGCUCCAGCAAUUCAGCACAUAUGUCUCAAGCAUACCACCUGUUUCCAAGAUGUAGUAGUGGAUGUGGA
UUGCGCCGAAAACACGAAAGAAGACCAGCUUGCCGAGAUUUCAUACCGGUUUCAAGGAAAAAAAGAAGCCGACCAGCC
GUGGAUAGUAGUGAAUACGUCCACGCUCUUCGAUGAACUGGAAUUGGACCCUCCUGAAAUUGAACCCGGAGUCCUGAA
AGUUUUGCGGACUGAGAAACAGUAUCUCGGUGUGUACAUCUGGAAUAUGAGGGGCAGUGAUGGAACAAGCACAUACGC
GACCUUUCUGGUGACAUGGAAAGGUGACGAAAAGACUAGAAACCCAACCCCAGCAGUGACCCCCCAACCUAGAGGUGC
UGAGUUCCACAUGUGGAAUUACCACUCCCAUGUAUUCUCUGUCGGAGACACCUUCAGCUUGGCGAUGCACCUCCAAUA
CAAGAUCCAUGAAGCCCCGUUCGACCUCUUGUUGGAGUGGCUCUACGUACCAAUAGACCCUACCUGUCAACCAAUGAG
GCUUUACUCUACAUGCCUUUACCACCCAAACGCACCACAAUGUCUUUCUCAUAUGAAUAGCGGUUGUACAUUUACAUC
UCCCCAUCUCGCGCAAAGAGUCGCGAGUACAGUUUACCAAAAUUGUGAGCAUGCGGACAAUUACACAGCAUAUUGUCU
UGGGAUCUCCCACAUGGAGCCCUCUUUUGGGCUUAUACUUCACGACGGGGGUACAACGCUUAAGUUCGUCGAUACGCC
AGAAUCCCUCUCUGGCUUGUAUGUAUUCGUGGUUUAUUUUAACGGUCACGUUGAGGCUGUGGCCUAUACUGUUGUUAG
CACUGUAGACCACUUUGUUAACGCCAUAGAAGAGAGGGGGUUCCCGCCCACCGCUGGCCAGCCACCGGCCACAACGAA
GCCUAAAGAGAUUACGCCGGUGAAUCCCGGCACAAGUCCCCUUAUCCGGUACGCCGCAUGGACAGGAGGACUGGCGGC
CGUCGUCCUCCUUUGUCUGGUUAUAUUCCUUAUAUGCACGGCAAAGCGAAUGCGGGUUAAGGCUUACCGAGUGGAUAA
AUCUCCAUAUAAUCAGAGCAUGUAUUACGCAGGGCUCCCUGUGGACGACUUUGAGGAUAGCGAGUCCACGGAUACAGA
AGAGGAAUUUGGAAACGCCAUCGGUGGGAGCCAUGGAGGGUCAUCUUACACCGUUUACAUUGACAAAACUAGAUGAua
accgcggugucaaaaaccgcguggacgugguuaacaucccugcugggaggaucagccguaauuuuauaauuuugcuug
gugcuggcuacuauuguggccauguacgugcugaccaaccagaaacauaauugaauacagcagcaauuggcaagcugc
uuacauagaacucgcggcgauuggcaugccgccuuaaaauuuuuauuuuauuuuuucuuuucuuuuccgaaucggauu
uuguuuuuaauauuucaaaaaaaaaaaaaaaaaaaaaaaaaaaaaaaaaaaaaaaaaaaaa
SEQ ID NO: 41
FL-gI-T2A-gE
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacguaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgcCGCCACCAU
GUUCCUUAUUCAAUGCCUCAUAUCCGCAGUCAUCUUCUAUAUUCAAGUUACUAAUGCACUUAUCUUCAAGGGCGAUCA
UGUCAGCCUGCAAGUGAAUUCAAGUCUUACGUCCAUUUUGAUUCCAAUGCAGAAUGAUAACUAUACCGAAAUUAAGGG
ACAGCUGGUUUUUAUUGGAGAACAACUGCCGACCGGUACAAACUACAGCGGGACCCUCGAGCUGCUUUACGCAGACAC
UGUGGCAUUCUGCUUUCGGUCAGUACAAGUUAUCAGAUACGACGGAUGCCCACGCAUUAGGACAUCUGCCUUCAUUUC
UUGCCGAUACAAGCAUAGUUGGCAUUACGGAAACUCUACCGAUAGAAUUUCAACUGAACCAGAUGCCGGUGUGAUGCU
CAAGAUAACCAAACCUGGGAUCAACGACGCAGGGGUGUAUGUCCUCUUGGUGAGAUUGGACCAUUCAAGGAGUACGGA
CGGGUUUAUACUGGGCGUGAACGUCUAUACCGCAGGAAGUCAUCAUAACAUUCACGGUGUCAUUUAUACCAGCCCCAG
UCUCCAGAAUGGGUACAGCACUCGAGCCCUGUUCCAGCAAGCAAGAUUGUGUGACCUUCCAGCCACUCCUAAGGGAUC
AGGCACAAGUCUUUUUCAACAUAUGUUGGAUCUCAGAGCAGGGAAAAGUCUUGAGGACAACCCGUGGCUCCAUGAAGA
CGUGGUUACUACUGAAACAAAGUCAGUGGUCAAGGAGGGAAUCGAGAACCAUGUGUACCCAACUGACAUGAGCACGCU
GCCUGAAAAAUCACUGAACGACCCACCAGAGAAUCUGCUGAUAAUAAUACCUAUUGUAGCGAGUGUUAUGAUUUUGAC
CGCAAUGGUCAUAGUUAUUGUAAUAAGCGUGAAAAGGAGACGAAUCAAAAAACAUCCGAUAUACAGGCCGAAUACGAA
GACAAGAAGAGGGAUUCAGAACGCGACUCCGGAGAGUGAUGUAAUGCUCGAAGCAGCCAUCGCUCAACUUGCCACCAU
UCGCGAAGAAAGCCCUCCGCAUUCCGUCGUAAAUCCUUUUGUCAAGCGGAGGAAAAGGGGAUCCGGGGAAGGCCGGGG
UAGUUUGCUGACGUGCGGAGAUGUUGAAGAGAACCCAGGGCCGAUGUUCUAUGAAGCUUUGAAGGCCGAACUUGUAUA
CACCCGAGCCGUGCAUGGUUUCCGCCCGAGAGCGAACUGCGUCGUACUGUCAGAUUACAUCCCCAGAGUCGCGUGCAA
CAUGGGUACGGUUAAUAAGCCCGUGGUGGGGGUGCUGAUGGGUUUUGGGAUAAUCACCGGAACUCUGCGCAUCACAAA
UCCUGUAAGAGCGUCAGUGCUGAGGUAUGACGAUUUCCACAUAGACGAGGACAAGCUGGAUACCAAUUCCGUUUACGA
ACCAUAUUAUCACUCCGAUCACGCCGAAUCCAGCUGGGUAAAUCGAGGAGAGUCCUCACGGAAGGCAUACGACCAUAA
CUCCCCAUAUAUAUGGCCUCGCAAUGAUUAUGACGGAUUCCUGGAAAACGCCCAUGAGCACCACGGCGUUUAUAACCA
GGGUCGCGGUAUCGAUUCUGGGGAAAGGUUGAUGCAACCAACUCAGAUGUCCGCUCAGGAAGAUUUGGGGGAUGACAC
CGGAAUCCAUGUGAUACCUACACUCAAUGGUGAUGAUCGACACAAAAUUGUAAACGUGGACCAGAGGCAAUACGGGGA
CGUUUUUAAGGGAGACCUGAAUCCAAAACCACAAGGACAACGACUCAUCGAGGUUUCUGUAGAAGAAAACCAUCCGUU
UACGCUCCGCGCCCCCAUACAGCGCAUAUAUGGCGUGCGCUAUACGGAGACCUGGUCUUUUCUCCCCAGUCUGACAUG
CACUGGUGAUGCAGCUCCAGCAAUUCAGCACAUAUGUCUCAAGCAUACCACCUGUUUCCAAGAUGUAGUAGUGGAUGU
GGAUUGCGCCGAAAACACGAAAGAAGACCAGCUUGCCGAGAUUUCAUACCGGUUUCAAGGAAAAAAAGAAGCCGACCA
GCCGUGGAUAGUAGUGAAUACGUCCACGCUCUUCGAUGAACUGGAAUUGGACCCUCCUGAAAUUGAACCCGGAGUCCU
GAAAGUUUUGCGGACUGAGAAACAGUAUCUCGGUGUGUACAUCUGGAAUAUGAGGGGCAGUGAUGGAACAAGCACAUA
CGCGACCUUUCUGGUGACAUGGAAAGGUGACGAAAAGACUAGAAACCCAACCCCAGCAGUGACCCCCCAACCUAGAGG
UGCUGAGUUCCACAUGUGGAAUUACCACUCCCAUGUAUUCUCUGUCGGAGACACCUUCAGCUUGGCGAUGCACCUCCA
AUACAAGAUCCAUGAAGCCCCGUUCGACCUCUUGUUGGAGUGGCUCUACGUACCAAUAGACCCUACCUGUCAACCAAU
GAGGCUUUACUCUACAUGCCUUUACCACCCAAACGCACCACAAUGUCUUUCUCAUAUGAAUAGCGGUUGUACAUUUAC
AUCUCCCCAUCUCGCGCAAAGAGUCGCGAGUACAGUUUACCAAAAUUGUGAGCAUGCGGACAAUUACACAGCAUAUUG
UCUUGGGAUCUCCCACAUGGAGCCCUCUUUUGGGCUUAUACUUCACGACGGGGGUACAACGCUUAAGUUCGUCGAUAC
GCCAGAAUCCCUCUCUGGCUUGUAUGUAUUCGUGGUUUAUUUUAACGGUCACGUUGAGGCUGUGGCCUAUACUGUUGU
UAGCACUGUAGACCACUUUGUUAACGCCAUAGAAGAGAGGGGGUUCCCGCCCACCGCUGGCCAGCCACCGGCCACAAC
GAAGCCUAAAGAGAUUACGCCGGUGAAUCCCGGCACAAGUCCCCUUAUCCGGUACGCCGCAUGGACAGGAGGACUGGC
GGCCGUCGUCCUCCUUUGUCUGGUUAUAUUCCUUAUAUGCACGGCAAAGCGAAUGCGGGUUAAGGCUUACCGAGUGGA
UAAAUCUCCAUAUAAUCAGAGCAUGUAUUACGCAGGGCUCCCUGUGGACGACUUUGAGGAUAGCGAGUCCACGGAUAC
AGAAGAGGAAUUUGGAAACGCCAUCGGUGGGAGCCAUGGAGGGUCAUCUUACACCGUUUACAUUGACAAAACUAGAUG
Auaaccgcggugucaaaaaccgcguggacgugguuaacaucccugcugggaggaucagccguaauuauuauaauuggc
uuggugcuggcuacuauuguggccauguacgugcugaccaaccagaaacauaauugaauacagcagcaauuggcaagc
ugcuuacauagaacucgcggcgauuggcaugccgccuuaaaauuuuuauuuuauuuuuucuuuucuuuuccgaaucgg
auuuuguuuuuaauauuucaaaaaaaaaaaaaaaaaaaaaaaaaaaaaaaaaaaaaaaaaaaaa
SEQ ID NO: 42
FL-gI-IRES-gE
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacguaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgcCGCCACCAU
GUUCCUUAUUCAAUGCCUCAUAUCCGCAGUCAUCUUCUAUAUUCAAGUUACUAAUGCACUUAUCUUCAAGGGCGAUCA
UGUCAGCCUGCAAGUGAAUUCAAGUCUUACGUCCAUUUUGAUUCCAAUGCAGAAUGAUAACUAUACCGAAAUUAAGGG
ACAGCUGGUUUUUAUUGGAGAACAACUGCCGACCGGUACAAACUACAGCGGGACCCUCGAGCUGCUUUACGCAGACAC
UGUGGCAUUCUGCUUUCGGUCAGUACAAGUUAUCAGAUACGACGGAUGCCCACGCAUUAGGACAUCUGCCUUCAUUUC
UUGCCGAUACAAGCAUAGUUGGCAUUACGGAAACUCUACCGAUAGAAUUUCAACUGAACCAGAUGCCGGUGUGAUGCU
CAAGAUAACCAAACCUGGGAUCAACGACGCAGGGGUGUAUGUCCUCUUGGUGAGAUUGGACCAUUCAAGGAGUACGGA
CGGGUUUAUACUGGGCGUGAACGUCUAUACCGCAGGAAGUCAUCAUAACAUUCACGGUGUCAUUUAUACCAGCCCCAG
UCUCCAGAAUGGGUACAGCACUCGAGCCCUGUUCCAGCAAGCAAGAUUGUGUGACCUUCCAGCCACUCCUAAGGGAUC
AGGCACAAGUCUUUUUCAACAUAUGUUGGAUCUCAGAGCAGGGAAAAGUCUUGAGGACAACCCGUGGCUCCAUGAAGA
CGUGGUUACUACUGAAACAAAGUCAGUGGUCAAGGAGGGAAUCGAGAACCAUGUGUACCCAACUGACAUGAGCACGCU
GCCUGAAAAAUCACUGAACGACCCACCAGAGAAUCUGCUGAUAAUAAUACCUAUUGUAGCGAGUGUUAUGAUUUUGAC
CGCAAUGGUCAUAGUUAUUGUAAUAAGCGUGAAAAGGAGACGAAUCAAAAAACAUCCGAUAUACAGGCCGAAUACGAA
GACAAGAAGAGGGAUUCAGAACGCGACUCCGGAGAGUGAUGUAAUGCUCGAAGCAGCCAUCGCUCAACUUGCCACCAU
UCGCGAAGAAAGCCCUCCGCAUUCCGUCGUAAAUCCUUUUGUCAAGUGACCCCUCUCCCUCCCCCCCCCCUAACGUUA
CUGGCCGAAGCCGCUUGGAAUAAGGCCGGUGUGCGUUUGUCUAUAUGUUAUUUUCCACCAUAUUGCCGUCUUUUGGCA
AUGUGAGGGCCCGGAAACCUGGCCCUGUCUUCUUGACGAGCAUUCCUAGGGGUCUUUCCCCUCUCGCCAAAGGAAUGC
AAGGUCUGUUGAAUGUCGUGAAGGAAGCAGUUCCUCUGGAAGCUUCUUGAAGACAAACAACGUCUGUAGCGACCCUUU
GCAGGCAGCGGAACCCCCCACCUGGCGACAGGUGCCUCUGCGGCCAAAAGCCACGUGUAUAAGAUACACCUGCAAAGG
CGGCACAACCCCAGUGCCACGUUGUGAGUUGGAUAGUUGUGGAAAGAGUCAAAUGGCUCUCCUCAAGCGUAUUCAACA
AGGGGCUGAAGGAUGCCCAGAAGGUACCCCAUUGUAUGGGAUCUGAUCUGGGGCCUCGGUGCACAUGCUUUACAUGUG
UUUAGUCGAGGUUAAAAAACGUCUAGGCCCCCCGAACCACGGGGACGUGGUUUUCCUUUGAAAAACACGAUGAUAAUA
UGGCCACAACCAUGUUCUAUGAAGCUUUGAAGGCCGAACUUGUAUACACCCGAGCCGUGCAUGGUUUCCGCCCGAGAG
CGAACUGCGUCGUACUGUCAGAUUACAUCCCCAGAGUCGCGUGCAACAUGGGUACGGUUAAUAAGCCCGUGGUGGGGG
UGCUGAUGGGUUUUGGGAUAAUCACCGGAACUCUGCGCAUCACAAAUCCUGUAAGAGCGUCAGUGCUGAGGUAUGACG
AUUUCCACAUAGACGAGGACAAGCUGGAUACCAAUUCCGUUUACGAACCAUAUUAUCACUCCGAUCACGCCGAAUCCA
GCUGGGUAAAUCGAGGAGAGUCCUCACGGAAGGCAUACGACCAUAACUCCCCAUAUAUAUGGCCUCGCAAUGAUUAUG
ACGGAUUCCUGGAAAACGCCCAUGAGCACCACGGCGUUUAUAACCAGGGUCGCGGUAUCGAUUCUGGGGAAAGGUUGA
UGCAACCAACUCAGAUGUCCGCUCAGGAAGAUUUGGGGGAUGACACCGGAAUCCAUGUGAUACCUACACUCAAUGGUG
AUGAUCGACACAAAAUUGUAAACGUGGACCAGAGGCAAUACGGGGACGUUUUUAAGGGAGACCUGAAUCCAAAACCAC
AAGGACAACGACUCAUCGAGGUUUCUGUAGAAGAAAACCAUCCGUUUACGCUCCGCGCCCCCAUACAGCGCAUAUAUG
GCGUGCGCUAUACGGAGACCUGGUCUUUUCUCCCCAGUCUGACAUGCACUGGUGAUGCAGCUCCAGCAAUUCAGCACA
UAUGUCUCAAGCAUACCACCUGUUUCCAAGAUGUAGUAGUGGAUGUGGAUUGCGCCGAAAACACGAAAGAAGACCAGC
UUGCCGAGAUUUCAUACCGGUUUCAAGGAAAAAAAGAAGCCGACCAGCCGUGGAUAGUAGUGAAUACGUCCACGCUCU
UCGAUGAACUGGAAUUGGACCCUCCUGAAAUUGAACCCGGAGUCCUGAAAGUUUUGCGGACUGAGAAACAGUAUCUCG
GUGUGUACAUCUGGAAUAUGAGGGGCAGUGAUGGAACAAGCACAUACGCGACCUUUCUGGUGACAUGGAAAGGUGACG
AAAAGACUAGAAACCCAACCCCAGCAGUGACCCCCCAACCUAGAGGUGCUGAGUUCCACAUGUGGAAUUACCACUCCC
AUGUAUUCUCUGUCGGAGACACCUUCAGCUUGGCGAUGCACCUCCAAUACAAGAUCCAUGAAGCCCCGUUCGACCUCU
UGUUGGAGUGGCUCUACGUACCAAUAGACCCUACCUGUCAACCAAUGAGGCUUUACUCUACAUGCCUUUACCACCCAA
ACGCACCACAAUGUCUUUCUCAUAUGAAUAGCGGUUGUACAUUUACAUCUCCCCAUCUCGCGCAAAGAGUCGCGAGUA
CAGUUUACCAAAAUUGUGAGCAUGCGGACAAUUACACAGCAUAUUGUCUUGGGAUCUCCCACAUGGAGCCCUCUUUUG
GGCUUAUACUUCACGACGGGGGUACAACGCUUAAGUUCGUCGAUACGCCAGAAUCCCUCUCUGGCUUGUAUGUAUUCG
UGGUUUAUUUUAACGGUCACGUUGAGGCUGUGGCCUAUACUGUUGUUAGCACUGUAGACCACUUUGUUAACGCCAUAG
AAGAGAGGGGGUUCCCGCCCACCGCUGGCCAGCCACCGGCCACAACGAAGCCUAAAGAGAUUACGCCGGUGAAUCCCG
GCACAAGUCCCCUUAUCCGGUACGCCGCAUGGACAGGAGGACUGGCGGCCGUCGUCCUCCUUUGUCUGGUUAUAUUCC
UUAUAUGCACGGCAAAGCGAAUGCGGGUUAAGGCUUACCGAGUGGAUAAAUCUCCAUAUAAUCAGAGCAUGUAUUACG
CAGGGCUCCCUGUGGACGACUUUGAGGAUAGCGAGUCCACGGAUACAGAAGAGGAAUUUGGAAACGCCAUCGGUGGGA
GCCAUGGAGGGUCAUCUUACACCGUUUACAUUGACAAAACUAGAUGAuaaccgcggugucaaaaaccgcguggacgug
guuaacaucccugcugggaggaucagccguaauuauuauaauuggcuuggugcuggcuacuauuguggccauguacgu
gcugaccaaccagaaacauaauugaauacagcagcaauuggcaagcugcuuacauagaacucgcggcgauuggcaugc
cgccuuaaaauuuuuauuuuauuuuuucuuuucuuuuccgaaucggauuuuguuuuuaauauuucaaaaaaaaaaaaa
aaaaaaaaaaaaaaaaaaaaaaaaaaaaaaaa
SEQ ID NO: 43
Secreted-gE
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacguaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgccaagAUGGG
UACCGUAAAUAAGCCCGUUGUCGGUGUCUUGAUGGGCUUUGGAAUCAUUACUGGAACUUUGAGGAUUACGAACCCUGU
CAGAGCCUCUGUUCUUCGGUACGACGACUUCCACAUUGACGAAGACAAGUUGGAUACCAACUCCGUAUACGAGCCGUA
UUACCACUCAGAUCAUGCCGAAUCAUCAUGGGUCAAUCGAGGCGAAUCUUCCCGAAAGGCGUACGAUCAUAACAGUCC
CUACAUAUGGCCCAGAAACGAUUACGACGGCUUUCUCGAGAACGCGCAUGAGCAUCAUGGGGUGUAUAAUCAAGGGAG
AGGCAUAGAUUCUGGUGAACGCUUGAUGCAACCAACUCAGAUGUCCGCACAAGAAGAUCUUGGGGACGACACAGGAAU
ACACGUCAUCCCUACACUGAACGGCGAUGAUCGGCAUAAGAUCGUAAAUGUGGAUCAACGGCAAUAUGGCGACGUGUU
UAAGGGGGACCUUAAUCCCAAGCCCCAGGGACAACGGUUGAUUGAGGUAUCUGUUGAGGAAAACCACCCCUUUACGCU
GCGGGCGCCGAUUCAGCGCAUCUACGGUGUUCGCUAUACGGAGACGUGGAGUUUCCUGCCAUCUCUCACAUGUACCGG
GGACGCUGCACCGGCAAUUCAACAUAUUUGCCUGAAACACACGACAUGCUUUCAAGACGUUGUAGUGGAUGUGGACUG
UGCGGAGAAUACCAAAGAGGACCAACUUGCGGAAAUAAGCUACCGAUUCCAAGGGAAGAAGGAGGCCGACCAGCCAUG
GAUUGUUGUUAAUACAUCUACACUUUUUGAUGAACUCGAACUUGACCCACCCGAGAUUGAACCUGGCGUUUUGAAAGU
CCUUAGAACAGAAAAACAAUACCUUGGUGUAUAUAUAUGGAACAUGAGGGGGUCCGACGGCACGUCUACCUAUGCCAC
CUUUCUGGUCACAUGGAAAGGAGAUGAAAAGACUCGCAAUCCUACCCCGGCGGUCACUCCUCAACCCAGAGGCGCUGA
GUUCCAUAUGUGGAAUUAUCAUUCACAUGUCUUCUCCGUUGGCGACACUUUUAGCUUGGCGAUGCAUCUGCAGUACAA
AAUACAUGAGGCGCCGUUCGACCUUCUCCUUGAAUGGCUCUAUGUGCCCAUUGAUCCUACUUGUCAACCGAUGAGGCU
UUACUCUACGUGCCUCUACCACCCGAAUGCGCCUCAAUGCCUGUCCCAUAUGAAUUCCGGGUGUACCUUUACAUCCCC
UCAUCUUGCUCAGCGAGUUGCUAGCACGGUUUACCAGAACUGCGAGCAUGCGGACAAUUAUACAGCCUACUGUCUUGG
CAUAAGUCACAUGGAACCUAGCUUCGGCCUGAUUCUCCAUGAUGGAGGAACAACGCUUAAAUUCGUAGACACCCCCGA
AUCAUUGAGCGGUUUGUAUGUUUUUGUAGUUUAUUUCAAUGGGCAUGUGGAGGCGGUAGCUUACACCGUCGUGUCAAC
AGUUGACCAUUUUGUCAACGCCAUCGAAGAAAGAGGAUUUCCGCCGACCGCGGGUCAGCCGCCCGCUACUACCAAACC
UAAGGAAAUUACUCCCGUGAACCCCGGCACCAGUCCGCUGAUAAGAUAUGCGGCUUGGACGGGAGGCUUGGCGugagg
cgcgccauacagcagcaauuggcaagcugcuuacauagaacucgcggcgauuggcaugccgccuuaaaauuuuuuuu
uauuuuuucuuuucuuuuccgaaucggauuuuguuuuuaauauuucaaaaaaaaaaaaaaaaaaaaaaaaaaaaaaaa
aaaaaaaaaaaaa

The following sequences (SEQ ID NOS: 44-47) are formatted to signify vector backbone and antigen open reading frames as follows: lower case letters signify the VEEV replicon backbone sequence; and UPPER CASE letters signify antigen open reading frame.

SEQ ID NO: 44
VEEV + Zika Envelope Protein Antigen Encoding RNA Sequence
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcuaugcugcuacgaugcaccgcgagggauucuugugcugcaaagugacagacacauugaacggggagagggucucuuu
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
acuaggacuacgagauagacaguuagucaugggguguuguugggcuuuuagaaggcacaagauaacaucuauuuauaag
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
ggugggcgagaaagcgccuuauuucuguggaggguuuauuuugugugacuccgugaccggcacagcgugccguguggca
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagAUGCGGAGAGGAGCAGACACAUC
CGUGGGAAUCGUGGGCCUGCUGCUGACCACAGCAAUGGCAGCCGAGGUGACCAGGAGAGGCAGCGCCUACUAUAUGUAC
CUGGACAGAAAUGAUGCCGGCGAGGCCAUCUCCUUUCCCACCACACUGGGCAUGAACAAGUGCUACAUCCAGAUCAUGG
ACCUGGGCCACAUGUGCGAUGCCACCAUGAGCUAUGAGUGUCCAAUGCUGGACGAGGGCGUGGAGCCCGACGAUGUGGA
UUGCUGGUGUAAUACCACAUCUACAUGGGUGGUGUACGGCACCUGUCACCACAAGAAGGGAGAGGCACGGCGCAGCAGG
AGAGCAGUGACACUGCCUUCCCACUCUACCAGGAAGCUGCAGACAAGAAGCCAGACCUGGCUGGAGUCCAGGGAGUAUA
CAAAGCACCUGAUCAGGGUGGAGAACUGGAUCUUUAGAAAUCCAGGAUUCGCACUGGCUGCCGCCGCCAUCGCAUGGCU
GCUGGGCAGCUCCACCAGCCAGAAAGUGAUCUACCUGGUCAUGAUCCUGCUGAUCGCCCCUGCCUAUUCUAUCCGGUGC
AUCGGCGUGAGCAAUAGGGACUUCGUGGAGGGAAUGUCCGGAGGCACCUGGGUGGAUGUGGUGCUGGAGCACGGCGGCU
GCGUGACAGUGAUGGCCCAGGACAAGCCAACCGUGGAUAUCGAGCUGGUGACCACAACCGUGUCCAACAUGGCCGAGGU
GAGGUCUUACUGCUAUGAGGCCAGCAUCUCCGACAUGGCCUCUGAUAGCAGAUGUCCCACCCAGGGCGAGGCCUACCUG
GACAAGCAGUCCGAUACACAGUACGUGUGCAAGCGGACCCUGGUGGACAGGGGAUGGGGAAAUGGAUGUGGCCUGUUUG
GCAAGGGCUCUCUGGUGACAUGCGCCAAGUUCGCCUGUAGCAAGAAGAUGACCGGCAAGUCCAUCCAGCCAGAGAACCU
GGAGUACCGGAUCAUGCUGUCUGUGCACGGCUCCCAGCACUCUGGCAUGAUCGUGAACGACACAGGCCACGAGACAGAU
GAGAAUCGGGCCAAGGUGGAGAUCACACCUAACUCCCCACGCGCCGAGGCCACCCUGGGAGGAUUUGGCUCUCUGGGCC
UGGACUGCGAGCCUAGGACAGGCCUGGACUUCUCCGAUCUGUACUAUCUGACCAUGAACAAUAAGCACUGGCUGGUGCA
CAAGGAGUGGUUUCACGACAUCCCACUGCCAUGGCACGCAGGAGCAGAUACAGGCACCCCACACUGGAACAAUAAGGAG
GCCCUGGUGGAGUUCAAGGACGCCCACGCCAAGCGGCAGACAGUGGUGGUGCUGGGCAGCCAGGAGGGAGCAGUGCACA
CCGCCCUGGCAGGCGCCCUGGAGGCCGAGAUGGACGGAGCAAAGGGCCGCCUGUCUAGCGGCCACCUGAAGUGCAGGCU
GAAGAUGGAUAAGCUGAGACUGAAGGGCGUGUCCUACUCUCUGUGCACAGCCGCCUUCACCUUCACCAAGAUCCCUGCC
GAGACACUGCACGGCACAGUGACCGUGGAGGUGCAGUAUGCCGGCACAGACGGCCCCUGUAAGGUGCCUGCCCAGAUGG
CCGUGGAUAUGCAGACACUGACCCCUGUGGGCAGGCUGAUCACCGCCAAUCCAGUGAUCACAGAGUCUACCGAGAACAG
CAAGAUGAUGCUGGAGCUGGACCCCCCUUUCGGCGAUAGCUAUAUCGUGAUCGGCGUGGGCGAGAAGAAGAUCACACAC
CACUGGCACAGAAGCGGCUCCACAAUCGGCAAGGCCUUUGAGGCAACCGUGCGGGGAGCAAAGAGGAUGGCCGUGCUGG
GCGACACCGCAUGGGAUUUCGGAUCUGUGGGAGGCGCCCUGAACAGCCUGGGCAAGGGCAUCCACCAGAUCUUCGGCGC
CGCCUUUAAGUCCCUGUUCGGCGGCAUGAGCUGGUUUUCCCAGAUCCUGAUCGGCACACUGCUGAUGUGGCUGGGCCUG
AACACCAAGAAUGGCUCUAUCAGCCUGAUGUGCCUGGCCCUGGGAGGCGUGCUGAUCUUCCUGUCCACCGCCGUGUCUG
CCugaccgcggugucaaaaaccgcguggacgugguuaacaucccugcugggaggaucagccguaauuauuauaauuggc
uuggugcuggcuacuauuguggccauguacgugcugaccaaccagaaacauaauugaauacagcagcaauuggcaagcu
gcuuacauagaacucgcggcgauuggcaugccgccuuaaaauuuuuauuuuauuuuuucuuuucuuuuccgaaucggau
uuuguuuuuaauauuucaaaaaaaaaaaaaaaaaaaaaaaaaaaaaaaaaaaaaaaaaaaaa
SEQ ID NO: 45
Full-length VEEV+ RSV-F Antigen Encoding RNA Sequence
auaggcggcgcaugagagaagcccagaccaauuaccuacccaaaauggagaaaguucacguugacaucgaggaagac
agcccauuccucagagcuuugcagcggagcuucccgcaguuugagguagaagccaagcaggucacugauaaugacca
ugcuaaugccagagcguuuucgcaucuggcuucaaaacugaucgaaacggagguggacccauccgacacgauccuug
acauuggaagugcgcccgcccgcagaauguauucuaagcacaaguaucauuguaucuguccgaugagaugugcggaa
gauccggacagauuguauaaguaugcaacuaagcugaagaaaaacuguaaggaaauaacugauaaggaauuggacaa
gaaaaugaaggagcuggccgccgucaugagcgacccugaccuggaaacugagacuaugugccuccacgacgacgagu
cgugucgcuacgaagggcaagucgcuguuuaccaggauguauacgcgguugacggaccgacaagucucuaucaccaa
gccaauaagggaguuagagucgccuacuggauaggcuuugacaccaccccuuuuauguuuaagaacuuggcuggagc
auauccaucauacucuaccaacugggccgacgaaaccguguuaacggcucguaacauaggccuaugcagcucugacg
uuauggagcggucacguagagggauguccauucuuagaaagaaguauuugaaaccauccaacaauguucuauucucu
guuggcucgaccaucuaccacgagaagagggacuuacugaggagcuggcaccugccgucuguauuucacuuacgugg
caagcaaaauuacacaugucggugugagacuauaguuaguugcgacggguacgucguuaaaagaauagcuaucaguc
caggccuguaugggaagccuucaggcuaugcugcuacgaugcaccgcgagggauucuugugcugcaaagugacagac
acauugaacggggagagggucucuuuucccgugugcacguaugugccagcuacauugugugaccaaaugacuggcau
acuggcaacagaugucagugcggacgacgcgcaaaaacugcugguugggcucaaccagcguauagucgucaacgguc
gcacccagagaaacaccaauaccaugaaaaauuaccuuuugcccguaguggcccaggcauuugcuaggugggcaaag
gaauauaaggaagaucaagaagaugaaaggccacuaggacuacgagauagacaguuagucaugggguguuguugggc
uuuuagaaggcacaagauaacaucuauuuauaagcgcccggauacccaaaccaucaucaaagugaacagcgauuucc
acucauucgugcugcccaggauaggcaguaacacauuggagaucgggcugagaacaagaaucaggaaaauguuagag
gagcacaaggagccgucaccucucauuaccgccgaggacguacaagaagcuaagugcgcagccgaugaggcuaagga
ggugcgugaagccgaggaguugcgcgcagcucuaccaccuuuggcagcugauguugaggagcccacucuggaggcag
acgucgacuugauguuacaagaggcuggggccggcucaguggagacaccucguggcuugauaaagguuaccagcuac
gauggcgaggacaagaucggcucuuacgcugugcuuucuccgcaggcuguacucaagagugaaaaauuaucuugcau
ccacccucucgcugaacaagucauagugauaacacacucuggccgaaaagggcguuaugccguggaaccauaccaug
guaaaguaguggugccagagggacaugcaauacccguccaggacuuucaagcucugagugaaagugccaccauugug
uacaacgaacgugaguucguaaacagguaccugcaccauauugccacacauggaggagcgcugaacacugaugaaga
auauuacaaaacugucaagcccagcgagcacgacggcgaauaccuguacgacaucgacaggaaacagugcgucaaga
aagaacuagucacugggcuagggcucacaggcgagcugguggauccucccuuccaugaauucgccuacgagagucug
agaacacgaccagccgcuccuuaccaaguaccaaccauagggguguauggcgugccaggaucaggcaagucuggcau
cauuaaaagcgcagucaccaaaaaagaucuaguggugagcgccaagaaagaaaacugugcagaaauuauaagggacg
ucaagaaaaugaaagggcuggacgucaaugccagaacuguggacucagugcucuugaauggaugcaaacaccccgua
gagacccuguauauugacgaagcuuuugcuugucaugcagguacucucagagcgcucauagccauuauaagaccuaa
aaaggcagugcucugcggggaucccaaacagugcgguuuuuuuaacaugaugugccugaaagugcauuuuaaccacg
agauuugcacacaagucuuccacaaaagcaucucucgccguugcacuaaaucugugacuucggucgucucaaccuug
uuuuacgacaaaaaaaugagaacgacgaauccgaaagagacuaagauugugauugacacuaccggcaguaccaaacc
uaagcaggacgaucucauucucacuuguuucagagggugggugaagcaguugcaaauagauuacaaaggcaacgaaa
uaaugacggcagcugccucucaagggcugacccguaaagguguguaugccguucgguacaaggugaaugaaaauccu
cuguacgcacccaccucagaacaugugaacguccuacugacccgcacggaggaccgcaucguguggaaaacacuagc
cggcgacccauggauaaaaacacugacugccaaguacccugggaauuucacugccacgauagaggaguggcaagcag
agcaugaugccaucaugaggcacaucuuggagagaccggacccuaccgacgucuuccagaauaaggcaaacgugugu
ugggccaaggcuuuagugccggugcugaagaccgcuggcauagacaugaccacugaacaauggaacacuguggauua
uuuugaaacggacaaagcucacucagcagagauaguauugaaccaacuaugcgugagguucuuuggacucgaucugg
acuccggucuauuuucugcacccacuguuccguuauccauuaggaauaaucacugggauaacuccccgucgccuaac
auguacgggcugaauaaagaagugguccgucagcucucucgcagguacccacaacugccucgggcaguugccacugg
aagagucuaugacaugaacacugguacacugcgcaauuaugauccgcgcauaaaccuaguaccuguaaacagaagac
ugccucaugcuuuaguccuccaccauaaugaacacccacagagugacuuuucuucauucgucagcaaauugaagggc
agaacuguccugguggucggggaaaaguuguccgucccaggcaaaaugguugacugguugucagaccggccugaggc
uaccuucagagcucggcuggauuuaggcaucccaggugaugugcccaaauaugacauaauauuuguuaaugugagga
ccccauauaaauaccaucacuaucagcagugugaagaccaugccauuaagcuuagcauguugaccaagaaagcuugu
cugcaucugaaucccggcggaaccugugucagcauagguuaugguuacgcugacagggccagcgaaagcaucauugg
ugcuauagcgcggcaguucaaguuuucccggguaugcaaaccgaaauccucacuugaagagacggaaguucuguuug
uauucauuggguacgaucgcaaggcccguacgcacaauccuuacaagcuuucaucaaccuugaccaacauuuauaca
gguuccagacuccacgaagccggaugugcacccucauaucauguggugcgaggggauauugccacggccaccgaagg
agugauuauaaaugcugcuaacagcaaaggacaaccuggcggaggggugugcggagcgcuguauaagaaauucccgg
aaagcuucgauuuacagccgaucgaaguaggaaaagcgcgacuggucaaaggugcagcuaaacauaucauucaugcc
guaggaccaaacuucaacaaaguuucggagguugaaggugacaaacaguuggcagaggcuuaugaguccaucgcuaa
gauugucaacgauaacaauuacaagucaguagcgauuccacuguuguccaccggcaucuuuuccgggaacaaagauc
gacuaacccaaucauugaaccauuugcugacagcuuuagacaccacugaugcagauguagccauauacugcagggac
aagaaaugggaaaugacucucaaggaagcaguggcuaggagagaagcaguggaggagauaugcauauccgacgacuc
uucagugacagaaccugaugcagagcuggugagggugcauccgaagaguucuuuggcuggaaggaagggcuacagca
caagcgauggcaaaacuuucucauauuuggaagggaccaaguuucaccaggcggccaaggauauagcagaaauuaau
gccauguggcccguugcaacggaggccaaugagcagguaugcauguauauccucggagaaagcaugagcaguauuag
gucgaaaugccccgucgaagagucggaagccuccacaccaccuagcacgcugccuugcuugugcauccaugccauga
cuccagaaagaguacagcgccuaaaagccucacguccagaacaaauuacugugugcucauccuuuccauugccgaag
uauagaaucacuggugugcagaagauccaaugcucccagccuauauuguucucaccgaaagugccugcguauauuca
uccaaggaaguaucucguggaaacaccaccgguagacgagacuccggagccaucggcagagaaccaauccacagagg
ggacaccugaacaaccaccacuuauaaccgaggaugagaccaggacuagaacgccugagccgaucaucaucgaagag
gaagaagaggauagcauaaguuugcugucagauggcccgacccaccaggugcugcaagucgaggcagacauucacgg
gccgcccucuguaucuagcucauccugguccauuccucaugcauccgacuuugauguggacaguuuauccauacuug
acacccuggagggagcuagcgugaccagcggggcaacgucagccgagacuaacucuuacuucgcaaagaguauggag
uuucuggcgcgaccggugccugcgccucgaacaguauucaggaacccuccacaucccgcuccgcgcacaagaacacc
gucacuugcacccagcagggccugcucgagaaccagccuaguuuccaccccgccaggcgugaauagggugaucacua
gagaggagcucgaggcgcuuaccccgucacgcacuccuagcaggucggucucgagaaccagccuggucuccaacccg
ccaggcguaaauagggugauuacaagagaggaguuugaggcguucguagcacaacaacaaugacgguuugaugcggg
ugcauacaucuuuuccuccgacaccggucaagggcauuuacaacaaaaaucaguaaggcaaacggugcuauccgaag
ugguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaac
agcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaaga
aauugcaaugucacgcaaaugagagaauugcccguauuggauucggcggccuuuaauguggaaugcuucaagaaaua
ugcguguaauaaugaauauugggaaacguuuaaagaaaaccccaucaggcuuacugaagaaaacgugguaaauuaca
uuaccaaauuaaaaggaccaaaagcugcugcucuuuuugcgaagacacauaauuugaauauguugcaggacauacca
auggacagguuuguaauggacuuaaagagagacgugaaagugacuccaggaacaaaacauacugaagaacggcccaa
gguacaggugauccaggcugccgauccgcuagcaacagcguaucugugcggaauccaccgagagcugguuaggagau
uaaaugcgguccugcuuccgaacauucauacacuguuugauaugucggcugaagacuuugacgcuauuauagccgag
cacuuccagccuggggauuguguucuggaaacugacaucgcgucguuugauaaaagugaggacgacgccauggcucu
gaccgcguuaaugauucuggaagacuuagguguggacgcagagcuguugacgcugauugaggcggcuuucggcgaaa
uuucaucaauacauuugcccacuaaaacuaaauuuaaauucggagccaugaugaaaucuggaauguuccucacacug
uuugugaacacagucauuaacauuguaaucgcaagcagaguguugagagaacggcuaaccggaucaccaugugcagc
auucauuggagaugacaauaucgugaaaggagucaaaucggacaaauuaauggcagacaggugcgccaccugguuga
auauggaagucaagauuauagaugcuguggugggcgagaaagcgccuuauuucuguggaggguuuauuuugugugac
uccgugaccggcacagcgugccguguggcagacccccuaaaaaggcuguuuaagcuuggcaaaccucuggcagcaga
cgaugaacaugaugaugacaggagaagggcauugcaugaagagucaacacgcuggaaccgaguggguauucuuucag
agcugugcaaggcaguagaaucaagguaugaaaccguaggaacuuccaucauaguuauggccaugacuacucuagcu
agcaguguuaaaucauucagcuaccugagaggggccccuauaacucucuacggcuaaccugaauggacuacgacaua
gucuaguccgccaagAUGGAGCUGCUCAUCCUUAAAGCGAAUGCUAUAACUACUAUACUUACAGCUGUAACCUUUUG
CUUCGCCUCUGGCCAAAACAUUACUGAGGAGUUUUAUCAAUCCACUUGCAGCGCAGUCUCAAAAGGAUAUCUGUCAG
CAUUGCGCACCGGGUGGUACACCAGCGUUAUCACUAUCGAGCUUUCCAACAUUAAAGAGAAUAAGUGUAACGGCACA
GACGCGAAAGUCAAACUCAUUAAGCAAGAACUUGAUAAAUACAAAAAUGCAGUUACUGAACUUCAGUUGCUCAUGCA
AUCAACCCCAGCAACGAACAAUAGGGCCAGGAGAGAAUUGCCGAGGUUUAUGAACUACACACUUAAUAACGCUAAAA
AGACUAACGUUACGCUCUCUAAGAAGCGGAAGCGCAGGUUUCUCGGGUUCCUUCUGGGGGUUGGCAGUGCAAUAGCA
UCCGGCGUCGCGGUAUCAAAGGUGCUUCAUCUUGAAGGAGAAGUUAACAAAAUCAAAAGCGCCUUGCUUUCAACUAA
UAAGGCAGUAGUAUCAUUGUCUAACGGUGUCAGCGUCUUGACUUCCAAGGUUUUGGAUUUGAAAAACUAUAUCGACA
AACAACUUCUCCCGAUCGUUAACAAGCAGUCAUGCAGUAUCUCCAACAUCGAAACCGUCAUCGAAUUCCAACAGAAA
AACAAUAGACUGCUUGAAAUUACUAGAGAGUUUUCAGUGAACGCUGGCGUUACCACUCCGGUAUCCACUUAUAUGCU
CACUAAUAGCGAACUGCUGUCUCUGAUAAAUGACAUGCCAAUAACUAACGACCAGAAGAAACUUAUGAGUAACAACG
UCCAGAUCGUGAGACAGCAAUCAUAUAGCAUUAUGAGCAUAAUUAAGGAGGAGGUCCUUGCAUACGUAGUCCAGCUC
CCACUGUACGGGGUUAUCGACACGCCAUGUUGGAAGCUUCAUACUUCCCCCUUGUGCACCACGAACACGAAGGAAGG
GUCUAACAUUUGUCUCACGCGCACUGAUCGGGGGUGGUACUGUGACAACGCCGGGUCAGUGUCAUUUUUCCCUCAGG
CCGAGACCUGCAAGGUCCAAUCAAACCGGGUAUUCUGUGAUACUAUGAACUCCCUGACUCUGCCUUCUGAAGUUAAC
CUGUGUAAUGUAGAUAUAUUCAAUCCUAAAUACGACUGCAAGAUAAUGACCAGCAAAACCGACGUGUCCUCAUCUGU
CAUCACUUCCCUUGGUGCUAUAGUAAGCUGCUAUGGCAAAACGAAAUGCACCGCGAGUAAUAAAAAUCGCGGUAUCA
UCAAGACAUUUAGUAACGGCUGCGAUUAUGUUUCCAACAAAGGUGUUGACACCGUAUCUGUGGGGAAUACCCUGUAU
UACGUAAAUAAGCAGGAAGGGAAAUCUCUCUACGUGAAGGGGGAACCGAUAAUCAACUUUUAUGACCCGCUGGUUUU
UCCGUCCGACGAAUUUGACGCGAGUAUCUCCCAAGUCAACGAGAAAAUUAACCAAAGCCUCGCGUUCAUAAGAAAAU
CCGAUGAGCUGCUGCAUAAUGUAAACGCGGGCAAAUCAACUACCAACAUCAUGAUUACAACAAUAAUCAUCGUGAUA
AUCGUGAUCCUGCUUUCACUUAUCGCUGUCGGGCUUCUUCUCUAUUGUAAGGCCCGGAGUACUCCCGUGACCUUGUC
UAAGGAUCAGCUCUCAGGUAUCAACAAUAUUGCAUUCAGCAAUUGAggcgcgccauacagcagcaauuggcaagcug
cuuacauagaacucgcggcgauuggcaugccgccuuaaaauuuuuauuuuauuuuuucuuuucuuuuccgaaucgga
uuuuguuuuuaauauuucaaaaaaaaaaaaaaaaaaaaaaaaaaaaaaaaaaaaaaaaaaaaa
SEQ ID NO: 46
Full-length VEEV + RSV-G Antigen Encoding RNA Sequence
auaggcggcgcaugagagaagcccagaccaauuaccuacccaaaauggagaaaguucacguugacaucgaggaagac
agcccauuccucagagcuuugcagcggagcuucccgcaguuugagguagaagccaagcaggucacugauaaugacca
ugcuaaugccagagcguuuucgcaucuggcuucaaaacugaucgaaacggagguggacccauccgacacgauccuug
acauuggaagugcgcccgcccgcagaauguauucuaagcacaaguaucauuguaucuguccgaugagaugugcggaa
gauccggacagauuguauaaguaugcaacuaagcugaagaaaaacuguaaggaaauaacugauaaggaauuggacaa
gaaaaugaaggagcuggccgccgucaugagcgacccugaccuggaaacugagacuaugugccuccacgacgacgagu
cgugucgcuacgaagggcaagucgcuguuuaccaggauguauacgcgguugacggaccgacaagucucuaucaccaa
gccaauaagggaguuagagucgccuacuggauaggcuuugacaccaccccuuuuauguuuaagaacuuggcuggagc
auauccaucauacucuaccaacugggccgacgaaaccguguuaacggcucguaacauaggccuaugcagcucugacg
uuauggagcggucacguagagggauguccauucuuagaaagaaguauuugaaaccauccaacaauguucuauucucu
guuggcucgaccaucuaccacgagaagagggacuuacugaggagcuggcaccugccgucuguauuucacuuacgugg
caagcaaaauuacacaugucggugugagacuauaguuaguugcgacggguacgucguuaaaagaauagcuaucaguc
caggccuguaugggaagccuucaggcuaugcugcuacgaugcaccgcgagggauucuugugcugcaaagugacagac
acauugaacggggagagggucucuuuucccgugugcacguaugugccagcuacauugugugaccaaaugacuggcau
acuggcaacagaugucagugcggacgacgcgcaaaaacugcugguugggcucaaccagcguauagucgucaacgguc
gcacccagagaaacaccaauaccaugaaaaauuaccuuuugcccguaguggcccaggcauuugcuaggugggcaaag
gaauauaaggaagaucaagaagaugaaaggccacuaggacuacgagauagacaguuagucaugggguguuguugggc
uuuuagaaggcacaagauaacaucuauuuauaagcgcccggauacccaaaccaucaucaaagugaacagcgauuucc
acucauucgugcugcccaggauaggcaguaacacauuggagaucgggcugagaacaagaaucaggaaaauguuagag
gagcacaaggagccgucaccucucauuaccgccgaggacguacaagaagcuaagugcgcagccgaugaggcuaagga
ggugcgugaagccgaggaguugcgcgcagcucuaccaccuuuggcagcugauguugaggagcccacucuggaggcag
acgucgacuugauguuacaagaggcuggggccggcucaguggagacaccucguggcuugauaaagguuaccagcuac
gauggcgaggacaagaucggcucuuacgcugugcuuucuccgcaggcuguacucaagagugaaaaauuaucuugcau
ccacccucucgcugaacaagucauagugauaacacacucuggccgaaaagggcguuaugccguggaaccauaccaug
guaaaguaguggugccagagggacaugcaauacccguccaggacuuucaagcucugagugaaagugccaccauugug
uacaacgaacgugaguucguaaacagguaccugcaccauauugccacacauggaggagcgcugaacacugaugaaga
auauuacaaaacugucaagcccagcgagcacgacggcgaauaccuguacgacaucgacaggaaacagugcgucaaga
aagaacuagucacugggcuagggcucacaggcgagcugguggauccucccuuccaugaauucgccuacgagagucug
agaacacgaccagccgcuccuuaccaaguaccaaccauagggguguauggcgugccaggaucaggcaagucuggcau
cauuaaaagcgcagucaccaaaaaagaucuaguggugagcgccaagaaagaaaacugugcagaaauuauaagggacg
ucaagaaaaugaaagggcuggacgucaaugccagaacuguggacucagugcucuugaauggaugcaaacaccccgua
gagacccuguauauugacgaagcuuuugcuugucaugcagguacucucagagcgcucauagccauuauaagaccuaa
aaaggcagugcucugcggggaucccaaacagugcgguuuuuuuaacaugaugugccugaaagugcauuuuaaccacg
agauuugcacacaagucuuccacaaaagcaucucucgccguugcacuaaaucugugacuucggucgucucaaccuug
uuuuacgacaaaaaaaugagaacgacgaauccgaaagagacuaagauugugauugacacuaccggcaguaccaaacc
uaagcaggacgaucucauucucacuuguuucagagggugggugaagcaguugcaaauagauuacaaaggcaacgaaa
uaaugacggcagcugccucucaagggcugacccguaaagguguguaugccguucgguacaaggugaaugaaaauccu
cuguacgcacccaccucagaacaugugaacguccuacugacccgcacggaggaccgcaucguguggaaaacacuagc
cggcgacccauggauaaaaacacugacugccaaguacccugggaauuucacugccacgauagaggaguggcaagcag
agcaugaugccaucaugaggcacaucuuggagagaccggacccuaccgacgucuuccagaauaaggcaaacgugugu
ugggccaaggcuuuagugccggugcugaagaccgcuggcauagacaugaccacugaacaauggaacacuguggauua
uuuugaaacggacaaagcucacucagcagagauaguauugaaccaacuaugcgugagguucuuuggacucgaucugg
acuccggucuauuuucugcacccacuguuccguuauccauuaggaauaaucacugggauaacuccccgucgccuaac
auguacgggcugaauaaagaagugguccgucagcucucucgcagguacccacaacugccucgggcaguugccacugg
aagagucuaugacaugaacacugguacacugcgcaauuaugauccgcgcauaaaccuaguaccuguaaacagaagac
ugccucaugcuuuaguccuccaccauaaugaacacccacagagugacuuuucuucauucgucagcaaauugaagggc
agaacuguccugguggucggggaaaaguuguccgucccaggcaaaaugguugacugguugucagaccggccugaggc
uaccuucagagcucggcuggauuuaggcaucccaggugaugugcccaaauaugacauaauauuuguuaaugugagga
ccccauauaaauaccaucacuaucagcagugugaagaccaugccauuaagcuuagcauguugaccaagaaagcuugu
cugcaucugaaucccggcggaaccugugucagcauagguuaugguuacgcugacagggccagcgaaagcaucauugg
ugcuauagcgcggcaguucaaguuuucccggguaugcaaaccgaaauccucacuugaagagacggaaguucuguuug
uauucauuggguacgaucgcaaggcccguacgcacaauccuuacaagcuuucaucaaccuugaccaacauuuauaca
gguuccagacuccacgaagccggaugugcacccucauaucauguggugcgaggggauauugccacggccaccgaagg
agugauuauaaaugcugcuaacagcaaaggacaaccuggcggaggggugugcggagcgcuguauaagaaauucccgg
aaagcuucgauuuacagccgaucgaaguaggaaaagcgcgacuggucaaaggugcagcuaaacauaucauucaugcc
guaggaccaaacuucaacaaaguuucggagguugaaggugacaaacaguuggcagaggcuuaugaguccaucgcuaa
gauugucaacgauaacaauuacaagucaguagcgauuccacuguuguccaccggcaucuuuuccgggaacaaagauc
gacuaacccaaucauugaaccauuugcugacagcuuuagacaccacugaugcagauguagccauauacugcagggac
aagaaaugggaaaugacucucaaggaagcaguggcuaggagagaagcaguggaggagauaugcauauccgacgacuc
uucagugacagaaccugaugcagagcuggugagggugcauccgaagaguucuuuggcuggaaggaagggcuacagca
caagcgauggcaaaacuuucucauauuuggaagggaccaaguuucaccaggcggccaaggauauagcagaaauuaau
gccauguggcccguugcaacggaggccaaugagcagguaugcauguauauccucggagaaagcaugagcaguauuag
gucgaaaugccccgucgaagagucggaagccuccacaccaccuagcacgcugccuugcuugugcauccaugccauga
cuccagaaagaguacagcgccuaaaagccucacguccagaacaaauuacugugugcucauccuuuccauugccgaag
uauagaaucacuggugugcagaagauccaaugcucccagccuauauuguucucaccgaaagugccugcguauauuca
uccaaggaaguaucucguggaaacaccaccgguagacgagacuccggagccaucggcagagaaccaauccacagagg
ggacaccugaacaaccaccacuuauaaccgaggaugagaccaggacuagaacgccugagccgaucaucaucgaagag
gaagaagaggauagcauaaguuugcugucagauggcccgacccaccaggugcugcaagucgaggcagacauucacgg
gccgcccucuguaucuagcucauccugguccauuccucaugcauccgacuuugauguggacaguuuauccauacuug
acacccuggagggagcuagcgugaccagcggggcaacgucagccgagacuaacucuuacuucgcaaagaguauggag
uuucuggcgcgaccggugccugcgccucgaacaguauucaggaacccuccacaucccgcuccgcgcacaagaacacc
gucacuugcacccagcagggccugcucgagaaccagccuaguuuccaccccgccaggcgugaauagggugaucacua
gagaggagcucgaggcgcuuaccccgucacgcacuccuagcaggucggucucgagaaccagccuggucuccaacccg
ccaggcguaaauagggugauuacaagagaggaguuugaggcguucguagcacaacaacaaugacgguuugaugcggg
ugcauacaucuuuuccuccgacaccggucaagggcauuuacaacaaaaaucaguaaggcaaacggugcuauccgaag
ugguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaac
agcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaaga
aauugcaaugucacgcaaaugagagaauugcccguauuggauucggcggccuuuaauguggaaugcuucaagaaaua
ugcguguaauaaugaauauugggaaacguuuaaagaaaaccccaucaggcuuacugaagaaaacgugguaaauuaca
uuaccaaauuaaaaggaccaaaagcugcugcucuuuuugcgaagacacauaauuugaauauguugcaggacauacca
auggacagguuuguaauggacuuaaagagagacgugaaagugacuccaggaacaaaacauacugaagaacggcccaa
gguacaggugauccaggcugccgauccgcuagcaacagcguaucugugcggaauccaccgagagcugguuaggagau
uaaaugcgguccugcuuccgaacauucauacacuguuugauaugucggcugaagacuuugacgcuauuauagccgag
cacuuccagccuggggauuguguucuggaaacugacaucgcgucguuugauaaaagugaggacgacgccauggcucu
gaccgcguuaaugauucuggaagacuuagguguggacgcagagcuguugacgcugauugaggcggcuuucggcgaaa
uuucaucaauacauuugcccacuaaaacuaaauuuaaauucggagccaugaugaaaucuggaauguuccucacacug
uuugugaacacagucauuaacauuguaaucgcaagcagaguguugagagaacggcuaaccggaucaccaugugcagc
auucauuggagaugacaauaucgugaaaggagucaaaucggacaaauuaauggcagacaggugcgccaccugguuga
auauggaagucaagauuauagaugcuguggugggcgagaaagcgccuuauuucuguggaggguuuauuuugugugac
uccgugaccggcacagcgugccguguggcagacccccuaaaaaggcuguuuaagcuuggcaaaccucuggcagcaga
cgaugaacaugaugaugacaggagaagggcauugcaugaagagucaacacgcuggaaccgaguggguauucuuucag
agcugugcaaggcaguagaaucaagguaugaaaccguaggaacuuccaucauaguuauggccaugacuacucuagcu
agcaguguuaaaucauucagcuaccugagaggggccccuauaacucucuacggcuaaccugaauggacuacgacaua
gucuaguccgccaagAUGUCCAAAAAUAAAGACCAGCGAACGGCCAAGACUCUGGAGAGGACUUGGGAUACCCUGAA
UCAUCUGUUGUUUAUUUCAAGUUGCCUUUAUAAAUUGAAUCUCAAAUCCGUCGCGCAAAUCACGCUGAGCAUACUCG
CAAUGAUUAUCUCAACUUCCUUGAUAAUUGCCGCCAUAAUCUUCAUUGCAAGUGCAAAUCAUAAAGUGACUCCUACA
ACCGCAAUAAUACAGGACGCGACCAGCCAAAUUAAGAACACGACUCCCACGUAUCUCACCCAAAAUCCCCAACUCGG
AAUUAGCCCAAGUAAUCCGUCAGAGAUUACUUCACAAAUCACCACCAUACUGGCGAGCACAACUCCAGGCGUAAAAU
CCACCCUCCAAUCUACUACCGUCAAGACUAAAAAUACCACUACUACUCAGACACAACCUAGUAAACCUACGACUAAG
CAGCGCCAGAAUAAACCCCCAAGCAAACCAAACAACGACUUCCAUUUUGAAGUUUUCAACUUCGUGCCUUGUUCUAU
CUGCUCUAAUAAUCCAACAUGCUGGGCCAUCUGCAAGCGCAUUCCGAAUAAGAAACCCGGUAAGAAAACUACAACGA
AACCUACCAAGAAGCCUACCCUGAAAACGACAAAGAAAGACCCGAAACCGCAAACAACCAAAAGUAAAGAAGUGCCC
ACAACUAAACCCACAGAAGAACCAACGAUAAACACGACCAAAACCAACAUAAUAACUACGCUGCUUACCAGCAACAC
GACAGGCAACCCGGAAUUGACCAGUCAGAUGGAAACUUUUCAUUCAACCAGCAGCGAAGGUAAUCCAAGUCCUAGUC
AGGUGUCUACGACAUCUGAAUAUCCAUCUCAGCCUAGUUCCCCUCCGAACACGCCGCGCCAGUGAUAAccgcggugu
caaaaaccgcguggacgugguuaacaucccugcugggaggaucagccguaauuauuauaauuggcuuggugcuggcu
acuauuguggccauguacgugcugaccaaccagaaacauaauugaauacagcagcaauuggcaagcugcuuacauag
aacucgcggcgauuggcaugccgccuuaaaauuuuuauuuuauuuuuucuuuucuuuuccgaaucggauuuuguuuu
uaauauuucaaaaaaaaaaaaaaaaaaaaaaaaaaaaaaaaaaaaaaaaaaaaa
SEQ ID NO: 47
Full Length VEEV + Influenza H3 Antigen Encoding RNA Sequence
auaggcggcgcaugagagaagcccagaccaauuaccuacccaaaauggagaaaguucacguugacaucgaggaagac
agcccauuccucagagcuuugcagcggagcuucccgcaguuugagguagaagccaagcaggucacugauaaugacca
ugcuaaugccagagcguuuucgcaucuggcuucaaaacugaucgaaacggagguggacccauccgacacgauccuug
acauuggaagugcgcccgcccgcagaauguauucuaagcacaaguaucauuguaucuguccgaugagaugugcggaa
gauccggacagauuguauaaguaugcaacuaagcugaagaaaaacuguaaggaaauaacugauaaggaauuggacaa
gaaaaugaaggagcuggccgccgucaugagcgacccugaccuggaaacugagacuaugugccuccacgacgacgagu
cgugucgcuacgaagggcaagucgcuguuuaccaggauguauacgcgguugacggaccgacaagucucuaucaccaa
gccaauaagggaguuagagucgccuacuggauaggcuuugacaccaccccuuuuauguuuaagaacuuggcuggagc
auauccaucauacucuaccaacugggccgacgaaaccguguuaacggcucguaacauaggccuaugcagcucugacg
uuauggagcggucacguagagggauguccauucuuagaaagaaguauuugaaaccauccaacaauguucuauucucu
guuggcucgaccaucuaccacgagaagagggacuuacugaggagcuggcaccugccgucuguauuucacuuacgugg
caagcaaaauuacacaugucggugugagacuauaguuaguugcgacggguacgucguuaaaagaauagcuaucaguc
caggccuguaugggaagccuucaggcuaugcugcuacgaugcaccgcgagggauucuugugcugcaaagugacagac
acauugaacggggagagggucucuuuucccgugugcacguaugugccagcuacauugugugaccaaaugacuggcau
acuggcaacagaugucagugcggacgacgcgcaaaaacugcugguugggcucaaccagcguauagucgucaacgguc
gcacccagagaaacaccaauaccaugaaaaauuaccuuuugcccguaguggcccaggcauuugcuaggugggcaaag
gaauauaaggaagaucaagaagaugaaaggccacuaggacuacgagauagacaguuagucaugggguguuguugggc
uuuuagaaggcacaagauaacaucuauuuauaagcgcccggauacccaaaccaucaucaaagugaacagcgauuucc
acucauucgugcugcccaggauaggcaguaacacauuggagaucgggcugagaacaagaaucaggaaaauguuagag
gagcacaaggagccgucaccucucauuaccgccgaggacguacaagaagcuaagugcgcagccgaugaggcuaagga
ggugcgugaagccgaggaguugcgcgcagcucuaccaccuuuggcagcugauguugaggagcccacucuggaggcag
acgucgacuugauguuacaagaggcuggggccggcucaguggagacaccucguggcuugauaaagguuaccagcuac
gauggcgaggacaagaucggcucuuacgcugugcuuucuccgcaggcuguacucaagagugaaaaauuaucuugcau
ccacccucucgcugaacaagucauagugauaacacacucuggccgaaaagggcguuaugccguggaaccauaccaug
guaaaguaguggugccagagggacaugcaauacccguccaggacuuucaagcucugagugaaagugccaccauugug
uacaacgaacgugaguucguaaacagguaccugcaccauauugccacacauggaggagcgcugaacacugaugaaga
auauuacaaaacugucaagcccagcgagcacgacggcgaauaccuguacgacaucgacaggaaacagugcgucaaga
aagaacuagucacugggcuagggcucacaggcgagcugguggauccucccuuccaugaauucgccuacgagagucug
agaacacgaccagccgcuccuuaccaaguaccaaccauagggguguauggcgugccaggaucaggcaagucuggcau
cauuaaaagcgcagucaccaaaaaagaucuaguggugagcgccaagaaagaaaacugugcagaaauuauaagggacg
ucaagaaaaugaaagggcuggacgucaaugccagaacuguggacucagugcucuugaauggaugcaaacaccccgua
gagacccuguauauugacgaagcuuuugcuugucaugcagguacucucagagcgcucauagccauuauaagaccuaa
aaaggcagugcucugcggggaucccaaacagugcgguuuuuuuaacaugaugugccugaaagugcauuuuaaccacg
agauuugcacacaagucuuccacaaaagcaucucucgccguugcacuaaaucugugacuucggucgucucaaccuug
uuuuacgacaaaaaaaugagaacgacgaauccgaaagagacuaagauugugauugacacuaccggcaguaccaaacc
uaagcaggacgaucucauucucacuuguuucagagggugggugaagcaguugcaaauagauuacaaaggcaacgaaa
uaaugacggcagcugccucucaagggcugacccguaaagguguguaugccguucgguacaaggugaaugaaaauccu
cuguacgcacccaccucagaacaugugaacguccuacugacccgcacggaggaccgcaucguguggaaaacacuagc
cggcgacccauggauaaaaacacugacugccaaguacccugggaauuucacugccacgauagaggaguggcaagcag
agcaugaugccaucaugaggcacaucuuggagagaccggacccuaccgacgucuuccagaauaaggcaaacgugugu
ugggccaaggcuuuagugccggugcugaagaccgcuggcauagacaugaccacugaacaauggaacacuguggauua
uuuugaaacggacaaagcucacucagcagagauaguauugaaccaacuaugcgugagguucuuuggacucgaucugg
acuccggucuauuuucugcacccacuguuccguuauccauuaggaauaaucacugggauaacuccccgucgccuaac
auguacgggcugaauaaagaagugguccgucagcucucucgcagguacccacaacugccucgggcaguugccacugg
aagagucuaugacaugaacacugguacacugcgcaauuaugauccgcgcauaaaccuaguaccuguaaacagaagac
ugccucaugcuuuaguccuccaccauaaugaacacccacagagugacuuuucuucauucgucagcaaauugaagggc
agaacuguccugguggucggggaaaaguuguccgucccaggcaaaaugguugacugguugucagaccggccugaggc
uaccuucagagcucggcuggauuuaggcaucccaggugaugugcccaaauaugacauaauauuuguuaaugugagga
ccccauauaaauaccaucacuaucagcagugugaagaccaugccauuaagcuuagcauguugaccaagaaagcuugu
cugcaucugaaucccggcggaaccugugucagcauagguuaugguuacgcugacagggccagcgaaagcaucauugg
ugcuauagcgcggcaguucaaguuuucccggguaugcaaaccgaaauccucacuugaagagacggaaguucuguuug
uauucauuggguacgaucgcaaggcccguacgcacaauccuuacaagcuuucaucaaccuugaccaacauuuauaca
gguuccagacuccacgaagccggaugugcacccucauaucauguggugcgaggggauauugccacggccaccgaagg
agugauuauaaaugcugcuaacagcaaaggacaaccuggcggaggggugugcggagcgcuguauaagaaauucccgg
aaagcuucgauuuacagccgaucgaaguaggaaaagcgcgacuggucaaaggugcagcuaaacauaucauucaugcc
guaggaccaaacuucaacaaaguuucggagguugaaggugacaaacaguuggcagaggcuuaugaguccaucgcuaa
gauugucaacgauaacaauuacaagucaguagcgauuccacuguuguccaccggcaucuuuuccgggaacaaagauc
gacuaacccaaucauugaaccauuugcugacagcuuuagacaccacugaugcagauguagccauauacugcagggac
aagaaaugggaaaugacucucaaggaagcaguggcuaggagagaagcaguggaggagauaugcauauccgacgacuc
uucagugacagaaccugaugcagagcuggugagggugcauccgaagaguucuuuggcuggaaggaagggcuacagca
caagcgauggcaaaacuuucucauauuuggaagggaccaaguuucaccaggcggccaaggauauagcagaaauuaau
gccauguggcccguugcaacggaggccaaugagcagguaugcauguauauccucggagaaagcaugagcaguauuag
gucgaaaugccccgucgaagagucggaagccuccacaccaccuagcacgcugccuugcuugugcauccaugccauga
cuccagaaagaguacagcgccuaaaagccucacguccagaacaaauuacugugugcucauccuuuccauugccgaag
uauagaaucacuggugugcagaagauccaaugcucccagccuauauuguucucaccgaaagugccugcguauauuca
uccaaggaaguaucucguggaaacaccaccgguagacgagacuccggagccaucggcagagaaccaauccacagagg
ggacaccugaacaaccaccacuuauaaccgaggaugagaccaggacuagaacgccugagccgaucaucaucgaagag
gaagaagaggauagcauaaguuugcugucagauggcccgacccaccaggugcugcaagucgaggcagacauucacgg
gccgcccucuguaucuagcucauccugguccauuccucaugcauccgacuuugauguggacaguuuauccauacuug
acacccuggagggagcuagcgugaccagcggggcaacgucagccgagacuaacucuuacuucgcaaagaguauggag
uuucuggcgcgaccggugccugcgccucgaacaguauucaggaacccuccacaucccgcuccgcgcacaagaacacc
gucacuugcacccagcagggccugcucgagaaccagccuaguuuccaccccgccaggcgugaauagggugaucacua
gagaggagcucgaggcgcuuaccccgucacgcacuccuagcaggucggucucgagaaccagccuggucuccaacccg
ccaggcguaaauagggugauuacaagagaggaguuugaggcguucguagcacaacaacaaugacgguuugaugcggg
ugcauacaucuuuuccuccgacaccggucaagggcauuuacaacaaaaaucaguaaggcaaacggugcuauccgaag
ugguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaac
agcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaaga
aauugcaaugucacgcaaaugagagaauugcccguauuggauucggcggccuuuaauguggaaugcuucaagaaaua
ugcguguaauaaugaauauugggaaacguuuaaagaaaaccccaucaggcuuacugaagaaaacgugguaaauuaca
uuaccaaauuaaaaggaccaaaagcugcugcucuuuuugcgaagacacauaauuugaauauguugcaggacauacca
auggacagguuuguaauggacuuaaagagagacgugaaagugacuccaggaacaaaacauacugaagaacggcccaa
gguacaggugauccaggcugccgauccgcuagcaacagcguaucugugcggaauccaccgagagcugguuaggagau
uaaaugcgguccugcuuccgaacauucauacacuguuugauaugucggcugaagacuuugacgcuauuauagccgag
cacuuccagccuggggauuguguucuggaaacugacaucgcgucguuugauaaaagugaggacgacgccauggcucu
gaccgcguuaaugauucuggaagacuuagguguggacgcagagcuguugacgcugauugaggcggcuuucggcgaaa
uuucaucaauacauuugcccacuaaaacuaaauuuaaauucggagccaugaugaaaucuggaauguuccucacacug
uuugugaacacagucauuaacauuguaaucgcaagcagaguguugagagaacggcuaaccggaucaccaugugcagc
auucauuggagaugacaauaucgugaaaggagucaaaucggacaaauuaauggcagacaggugcgccaccugguuga
auauggaagucaagauuauagaugcuguggugggcgagaaagcgccuuauuucuguggaggguuuauuuugugugac
uccgugaccggcacagcgugccguguggcagacccccuaaaaaggcuguuuaagcuuggcaaaccucuggcagcaga
cgaugaacaugaugaugacaggagaagggcauugcaugaagagucaacacgcuggaaccgaguggguauucuuucag
agcugugcaaggcaguagaaucaagguaugaaaccguaggaacuuccaucauaguuauggccaugacuacucuagcu
agcaguguuaaaucauucagcuaccugagaggggccccuauaacucucuacggcuaaccugaauggacuacgacaua
gucuaguccgccaagAUGAAAACAAUUAUCGCUCUCAGUUAUAUUUUCUGCCUUCCUUUGGGGCAAGACUUGCCGGG
CAAUGAUAAUAGUACAGCGACUCUCUGUCUCGGACACCACGCAGUACCGAACGGCACACUUGUGAAGACAAUCACAG
ACGAUCAAAUCGAAGUGACGAAUGCAACAGAGCUUGUUCAAUCAUCAUCUACCGGCAAAAUCUGCAAUAAUCCGCAU
AGAAUCCUUGAUGGAAUCGACUGCACCCUGAUUGACGCACUGUUGGGAGAUCCUCAUUGCGAUGUGUUUCAGAAUGA
GACAUGGGACCUUUUUGUCGAGAGGAGCAAAGCGUUUUCCAACUGCUACCCGUAUGACGUGCCUGAUUACGCGUCAC
UCCGCUCACUUGUAGCAUCAAGCGGUACUCUGGAGUUCAUCACCGAAGGAUUCACCUGGACGGGCGUAACUCAGAAC
GGCGGUUCUAACGCAUGUAAGAGGGGGCCAGGGUCCGGCUUCUUCUCACGGCUCAACUGGUUGACUAAGUCUGGUUC
AACAUACCCGGUCCUUAACGUUACAAUGCCGAAUAAUGACAACUUUGAUAAACUCUACAUCUGGGGCAUUCACCAUC
CAUCCACAAAUCAAGAACAGACAAGUUUGUACGUUCAGGCGUCAGGGCGCGUUACCGUGAGUACAAGAAGAUCACAG
CAAACAAUUAUACCCAAUAUUGGGUCCCGACCCUGGGUAAGAGGACUGUCCUCUCGCAUCUCCAUAUACUGGACCAU
UGUCAAACCGGGCGACGUCCUGGUUAUCAAUAGUAAUGGAAACCUUAUUGCUCCGCGCGGCUAUUUCAAAAUGCGAA
CUGGAAAGUCAAGUAUAAUGCGCUCAGACGCACCGAUCGAUACUUGUAUCAGUGAAUGCAUCACCCCUAACGGGUCC
AUACCGAACGAUAAGCCCUUCCAGAAUGUGAAUAAAAUCACGUAUGGAGCAUGCCCCAAAUACGUGAAGCAAAACAC
CCUCAAGUUGGCUACGGGUAUGCGCAACGUCCCAGAAAAACAAACGCGAGGCUUGUUUGGGGCGAUAGCAGGUUUUA
UCGAGAACGGCUGGGAAGGAAUGAUCGAUGGGUGGUACGGCUUUCGCCAUCAAAACUCAGAAGGAACUGGGCAGGCC
GCAGAUCUUAAGUCUACGCAAGCGGCGAUAGAUCAAAUUAAUGGCAAGUUGAAUAGGGUGAUAGAGAAGACGAACGA
GAAGUUCCAUCAAAUAGAGAAAGAAUUCAGUGAAGUAGAGGGGCGAAUUCAGGAUUUGGAAAAAUAUGUCGAGGAUA
CUAAGAUCGACCUGUGGAGCUAUAACGCAGAGCUUCUGGUAGCACUUGAGAACCAGCAUACUAUUGAUCUCACCGAU
UCCGAGAUGAACAAGCUUUUUGAGAAGACCAGGAGACAGUUGCGCGAGAAUGCCGAAGAAAUGGGUAACGGUUGUUU
UAAGAUUUAUCACAAAUGUGACAACGCGUGUAUUGAGUCCAUUAGGAAUGGUACAUAUGAUCACGAUGUGUAUCGCG
AUGAAGCACUUAACAAUCGGUUCCAAAUAAAGGGCGUGGAGCUCAAGAGUGGGUAUAAAGAUUGGAUCCUCUGGAUC
UCAUUCGCGAUUUCCUGCUUCCUCCUGUGCGUUGUCUUGCUGGGCUUUAUUAUGUGGGCAUGUCAGAGAGGUAACAU
CCGGUGCAACAUAUGUAUCUGAUAAccgcggugucaaaaaccgcguggacgugguuaacaucccugcugggaggauc
agccguaauuauuauaauuggcuuggugcuggcuacuauuguggccauguacgugcugaccaaccagaaacauaauu
uuuuucuuuucuuuuccgaaucggauuuuguuuuuaauauuucaaaaaaaaaaaaaaaaaaaaaaaaaaaaaaaaaa
aaaaaaaaaaa
SEQ ID NO: 48
Vector Backbone RNA Sequence 1
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagnugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaaa
SEQ ID NO: 49
Vector Backbone RNA Sequence 2
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggccggcaguucaaguuuucccggguaugca
aaccgaaauccucacuugaagagacggaaguucuguuuguauucauuggguacgaucgcaaggcccguacgcacaaccuuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagnugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaaagcugcuuacaugaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaaa
SEQ ID NO: 50
Vector Backbone RNA Sequence 3
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagnuaguaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaaa
SEQ ID NO: 51
Vector Backbone RNA Sequence 4
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucugcggcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagnugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaaa
SEQ ID NO: 52
Vector Backbone RNA Sequence 5
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuacoggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagnugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaaa
SEQ ID NO: 53
Vector Backbone RNA Sequence 6
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuacoggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGAGAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGACAAUCCAGUGCUUCCAUUUAAUGAUGGGGU
UUACUUUGCCAGUAUCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGACGUGUA
UUACCAUAAGAAUAAUAAAUCCUGGAUGGAGUCUGGGGUUUAUAGUAGUGCUAAUAAUUGCACUUUCGAAUACGUGUCCCA
ACCAUUCCUCAUGGACCUUGAGGGCAAACAGGGGAAUUUUAAAAACUUGCGCGAAUUUGUCUUUAAGAAUAUCGACGGAUA
CUUUAAGAUCUAUAGUAAACACACUCCUAUCAACCUCGUUCGGGAUCUUCCCCAAGGCUUUUCUGCUCUCGAACCCCUCGU
AGACUUGCCAAUUGGGAUAAAUAUCACUCGCUUUCAAACUUUGCUUGCCCUCCACAGGAGCUACCUGACACCCGGCGACUC
UUCUUCUGGUUUGACCGCCGGCGCCGCUGCCUAUUAUGUUGGUUACCUUCAGCCACGAACAUUCUUGCUCAAGUAUAACGA
GAAUGGCACCAUUACCGACGCCGUCGAUUGUGCAUUGGAUCCCUUGUCUGAAACAAAAUGUACCUUGAAGUCCUUUACCGU
AGAGAAAGGCAUAUACCAGACUUCCAACUUCCGAGUUCAGCCUACAGAAUCCAUCGUACGAUUUCCCAACAUCACAAACCU
CUGCCCUUUCGGUGAAGUAUUUAAUGCUACACGCUUCGCUUCAGUCUAUGCCUGGAAUAGGAAGCGCAUAUCAAAUUGCGU
GGCCGAUUAUUCAGUCCUCUAUAAUAGCGCAUCCUUCAGUACUUUCAAGUGCUACGGCGUUUCCCCCACCAAACUCAAUGA
UCUUUGCUUCACCAACGUCUAUGCUGACAGUUUUGUCAUACGAGGCGACGAAGUACGCCAGAUUGCCCCCGGGCAGACAGG
UAAUAUUGCUGAUUAUAAUUAUAAACUCCCAGAUGACUUUACUGGAUGCGUCAUAGCCUGGAAUUCCAACAAUCUAGAUUC
CAAGGUUGGUGGGAAUUAUAAUUACCGUUAUCGACUGUUCAGAAAGAGUAACUUGAAACCAUUUGAGAGAGACAUAUCCAC
CGAGAUUUACCAGGCAGGCAGUAAGCCUUGUAACGGCGUUGAGGGAUUUAACUGCUAUUUUCCUUUGCAAUCCUAUGGCUU
UCAACCAACAAACGGGGUUGGCUAUCAACCCUAUCGAGUGGUUGUCCUCAGCUUUGAACUUUUGCACGCUCCCGCCACAGU
CUGCGGACCAAAAAAGAGUACAAAUCUUGUCAAGAAUAAGUGCGUAAAUUUCAAUUUCAAUGGCCUUACAGGAACAGGCGU
GCUGACUGAGUCAAACAAGAAUUUCCUGCCAUUUCAGCAGUUUGGGCGGGAUAUAGCAGACACAACUGACGCUGUACGCGA
UCCUCAGACUUUGGAGAUCUUGGACAUCACUCCCUGUUCUUUCGGAGGGGUAUCUGUCAUCACCCCCGGAACUAAUACAUC
AAAUCAGGUCGCUGUGUUGUACCAAGGUGUCAACUGCACAGAAGUCCCCGUUGCUAUACACGCAGACCAGCUCACCCCCAC
AUGGCGGGUGUACUCAACUGGCUCAAACGUAUUCCAGACCAGAGCUGGGUGCUUGAUCGGUGCUGAACACGUGAACAAUAG
CUAUGAAUGCGAUAUUCCCAUCGGUGCCGGGAUCUGCGCUAGCUAUCAGACACAGACCAAUUCCCGCAGGCGGGCUCGCUC
UGUAGCAUCCCAGUCUAUUAUUGCCUACACUAUGUCAUUGGGCGCCGAGAAUAGCGUCGCAUAUUCAAAUAAUUCUAUUGC
AAUACCCACCAACUUCACAAUCUCCGUAACUACAGAAAUACUUCCAGUUUCCAUGACAAAGACAUCAGUGGAUUGUACAAU
GUAUAUAUGCGGAGAUUCCACAGAAUGUUCAAAUUUGCUCUUGCAGUACGGCUCCUUCUGCACCCAGCUCAACAGGGCACU
UACAGGUAUUGCUGUCGAACAGGACAAGAACACACAAGAAGUCUUCGCCCAAGUCAAACAGAUAUACAAAACUCCUCCCAU
AAAGGAUUUUGGCGGCUUCAACUUUAGUCAGAUCCUCCCAGACCCUUCAAAACCAUCUAAACGAUCAUUUAUUGAAGAUCU
GCUGUUCAACAAGGUCACUCUUGCCGAUGCUGGAUUCAUUAAGCAAUACGGUGACUGCCUUGGUGAUAUUGCUGCCCGAGA
UCUGAUCUGUGCCCAGAAAUUCAACGGGCUCACUGUACUCCCUCCACUGCUCACAGACGAAAUGAUUGCACAGUACACAAG
UGCCCUGUUGGCAGGCACAAUCACUAGCGGCUGGACCUUUGGCGCAGGUGCAGCACUCCAAAUACCUUUUGCCAUGCAGAU
GGCCUAUCGGUUUAAUGGGAUAGGCGUGACUCAAAAUGUCCUCUACGAAAACCAAAAGUUGAUAGCUAACCAAUUCAAUUC
AGCAAUCGGGAAGAUACAGGAUUCACUGUCUAGUACUGCUAGUGCCCUUGGUAAGCUGCAGAACGUUGUCAACCAGAAUGC
UCAAGCUCUGAAUACAUUGGUUAAGCAGCUCUCUAGUAAUUUUGGGGCCAUCUCUUCAGUACUUAAUGAUAUUUUGAGCCG
AUUGGACCCACCUGAAGCUGAAGUACAGAUCGACAGGCUGAUAACAGGCCGGCUCCAAUCCCUCCAAACAUACGUGACACA
ACAACUCAUACGCGCAGCCGAAAUCCGAGCCAGCGCUAACCUGGCAGCUACCAAGAUGUCAGAAUGCGUUCUGGGCCAGAG
UAAACGCGUAGAUUUCUGCGGGAAAGGGUACCACCUGAUGUCCUUUCCACAAUCUGCACCUCACGGGGUCGUCUUUUUGCA
UGUAACAUACGUACCCGCACAAGAGAAGAAUUUUACUACCGCUCCUGCCAUCUGUCAUGACGGGAAAGCUCAUUUUCCUCG
CGAAGGUGUGUUUGUAUCUAAUGGUACACAUUGGUUUGUCACACAGCGGAAUUUCUAUGAACCCCAGAUCAUUACAACUGA
CAACACUUUUGUUUCCGGGAAUUGUGACGUGGUCAUAGGAAUCGUAAAUAACACUGUAUAUGAUCCCCUCCAACCAGAGCU
GGACUCUUUUAAAGAAGAACUGGAUAAAUAUUUCAAGAACCACACAAGUCCCGACGUGGACCUUGGGGACAUAAGUGGUAU
UAACGCAUCUGUGGUUAACAUUCAAAAGGAAAUCGACAGACUCAACGAGGUGGCCAAAAACCUGAACGAAAGCUUGAUAGA
UCUCCAGGAGUUGGGCAAGUAUGAACAGUACAUUAAAUGGCCAUGGUACAUAUGGCUUGGCUUUAUCGCUGGCCUUAUCGC
CAUCGUAAUGGUUACAAUCAUGCUGUGCUGCAUGACCUCCUGCUGUUCUUGUUUGAAAGGGUGUUGUUCUUGUGGUAGUUG
UUGCAAGUUUGACGAAGAUGAUUCCGAACCUGUUCUUAAGGGGGUAAAGCUUCACUAUACAUGAuaaccgcggugucaaaa
accgcguggacgugguuaacaucccugcugggaggaucagccguaauuauuauaauuggcuuggugcuggcuacuauugug
gccauguacgugcugaccaaccagaaacauaauugaauacagcagcaauuggcaagcugcuuacauagaacucgcggcgau
uggcaugccgccuuaaaauuuuuauuuuauuuuuucuuuucuuuuccgaaucggauuuuguuuuuaauauuucaaaaaa
aaaaaaaaaaaaaaaaaaaaaaaaaaaaaaaaaaaaaaa
SEQ ID NO: 54
Vector Backbone RNA Sequence 7
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggauguccauucuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguuaguugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugccagcua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugucaugcaggua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguucuuuggcuggaaggaagggcuaca
gcacaagcgauggcaaaacuuucucauauuuggaagggaccaaguuucaccaggcggccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccagcgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccgccaccnuaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaaa

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A composition comprising:
a lipid carrier, wherein the lipid carrier comprises:
liquid oil; and
surfactants, wherein the surfactants comprise:
a cationic lipid;
a hydrophilic surfactant; and
a hydrophobic surfactant; and
at least one nucleic acid encoding an antigen sequence, wherein the antigen sequence comprises a sequence encoding for a viral antigen sequence from an influenza virus.

2. The composition of claim 1, wherein the influenza virus is influenza A or influenza B.

3. The composition of claim 1, wherein the at least one nucleic acid comprises an antigen sequence encoding for an influenza hemagglutinin protein stem region or a functional variant thereof.

4. The composition of claim 1, wherein the at least one nucleic acid comprises a sequence at least 85% identical to SEQ ID NO: 3.

5. The composition of claim 1, wherein the at least one nucleic acid comprises a sequence encoding for an amino acid sequence of any one of SEQ ID NOS: 10-12.

6. The composition of claim 1, wherein the nucleic acid is in complex with the lipid carrier.

7. The composition of claim 1, wherein, the nucleic acid further encodes for an RNA polymerase.

8. The composition of claim 7, wherein the RNA polymerase is a Venezuelan equine encephalitis virus (VEEV) RNA polymerase.

9. The composition of claim 8, wherein the nucleic acid encoding for the RNA polymerase comprises the nucleic acid sequence of SEQ ID NO: 34.

10. The composition of claim 1, wherein the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, or vitamin E.

11. The composition of claim 10, wherein the triglyceride is capric triglyceride, caprylic triglyceride, a caprylic and capric triglyceride, a triglyceride ester, or myristic acid triglycerin.

12. The composition of claim 1, wherein the lipid carrier comprises a hydrophobic core.

13. The composition of claim 1, wherein the lipid carrier comprises an inorganic particle.

14. The composition of claim 13, wherein the inorganic particle is within a hydrophobic core of the lipid carrier.

15. The composition of claim 13, wherein the inorganic particle comprises a metal.

16. The composition of claim 15, wherein the metal comprises a metal salt, a metal oxide, a metal hydroxide, or a metal phosphate.

17. The composition of claim 16, wherein the metal oxide comprises aluminum oxide, aluminum oxyhydroxide, iron oxide, titanium dioxide, or silicon dioxide.

18. The composition of claim 1, wherein the hydrophobic surfactant is sorbitan monolaurate, sorbitan monopalmitate, sorbitan monostearate, sorbitan monooleate, or sorbitan trioleate.

19. The composition of claim 1, wherein the hydrophilic surfactant is a polysorbate.

20. The composition of claim 1, wherein the at least one nucleic acid is present in an amount of up to about 5, about 10, about 25, about 50, or about 100 micrograms (μg).

21. The composition of claim 1, wherein cationic lipid comprises 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP), dipalmitoyl(C16:0)trimethyl ammonium propane (DPTAP), distearoyltrimethylammonium propane (DSTAP), N-[l-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; or TT3, N1,N3,N5-tris(3-(didodecylamino)propyl)benzene-1,3,5-tricarboxamide.


SEQUENCE LISTING (54 sequences)

SEQ ID NO: 1 - RNA, 900 nt, Human orthopneumovirus
  source 1..900: mol_type = other RNA
atgtccaaaaataaagaccagcgaacggccaagactctggagaggacttgggataccctgaatcatctgttgtttatttcaagttgcctttataaattgaatctcaaatccgtcgcgcaaatcacgctgagcatactcgcaatgattatctcaacttccttgataattgccgccataatcttcattgcaagtgcaaatcataaagtgactcctacaaccgcaataatacaggacgcgaccagccaaattaagaacacgactcccacgtatctcacccaaaatccccaactcggaattagcccaagtaatccgtcagagattacttcacaaatcaccaccatactggcgagcacaactccaggcgtaaaatccaccctccaatctactaccgtcaagactaaaaataccactactactcagacacaacctagtaaacctacgactaagcagcgccagaataaacccccaagcaaaccaaacaacgacttccattttgaagttttcaacttcgtgccttgttctatctgctctaataatccaacatgctgggccatctgcaagcgcattccgaataagaaacccggtaagaaaactacaacgaaacctaccaagaagcctaccctgaaaacgacaaagaaagacccgaaaccgcaaacaaccaaaagtaaagaagtgcccacaactaaacccacagaagaaccaacgataaacacgaccaaaaccaacataataactacgctgcttaccagcaacacgacaggcaacccggaattgaccagtcagatggaaacttttcattcaaccagcagcgaaggtaatccaagtcctagtcaggtgtctacgacatctgaatatccatctcagcctagttcccctccgaacacgccgcgccagtgataa

SEQ ID NO: 2 - RNA, 1725 nt, Human orthopneumovirus
  source 1..1725: mol_type = other RNA
atggagctgctcatccttaaagcgaatgctataactactatacttacagctgtaaccttttgcttcgcctctggccaaaacattactgaggagttttatcaatccacttgcagcgcagtctcaaaaggatatctgtcagcattgcgcaccgggtggtacaccagcgttatcactatcgagctttccaacattaaagagaataagtgtaacggcacagacgcgaaagtcaaactcattaagcaagaacttgataaatacaaaaatgcagttactgaacttcagttgctcatgcaatcaaccccagcaacgaacaatagggccaggagagaattgccgaggtttatgaactacacacttaataacgctaaaaagactaacgttacgctctctaagaagcggaagcgcaggtttctcgggttccttctgggggttggcagtgcaatagcatccggcgtcgcggtatcaaaggtgcttcatcttgaaggagaagttaacaaaatcaaaagcgccttgctttcaactaataaggcagtagtatcattgtctaacggtgtcagcgtcttgacttccaaggttttggatttgaaaaactatatcgacaaacaacttctcccgatcgttaacaagcagtcatgcagtatctccaacatcgaaaccgtcatcgaattccaacagaaaaacaatagactgcttgaaattactagagagttttcagtgaacgctggcgttaccactccggtatccacttatatgctcactaatagcgaactgctgtctctgataaatgacatgccaataactaacgaccagaagaaacttatgagtaacaacgtccagatcgtgagacagcaatcatatagcattatgagcataattaaggaggaggtccttgcatacgtagtccagctcccactgtacggggttatcgacacgccatgttggaagcttcatacttcccccttgtgcaccacgaacacgaaggaagggtctaacatttgtctcacgcgcactgatcgggggtggtactgtgacaacgccgggtcagtgtcatttttccctcaggccgagacctgcaaggtccaatcaaaccgggtattctgtgatactatgaactccctgactctgccttctgaagttaacctgtgtaatgtagatatattcaatcctaaatacgactgcaagataatgaccagcaaaaccgacgtgtcctcatctgtcatcacttcccttggtgctatagtaagctgctatggcaaaacgaaatgcaccgcgagtaataaaaatcgcggtatcatcaagacatttagtaacggctgcgattatgtttccaacaaaggtgttgacaccgtatctgtggggaataccctgtattacgtaaataagcaggaagggaaatctctctacgtgaagggggaaccgataatcaacttttatgacccgctggtttttccgtccgacgaatttgacgcgagtatctcccaagtcaacgagaaaattaaccaaagcctcgcgttcataagaaaatccgatgagctgctgcataatgtaaacgcgggcaaatcaactaccaacatcatgattacaacaataatcatcgtgataatcgtgatcctgctttcacttatcgctgtcgggcttcttctctattgtaaggcccggagtactcccgtgaccttgtctaaggatcagctctcaggtatcaacaatattgcattcagcaattga

SEQ ID NO: 3 - RNA, 1704 nt, Influenza A virus
  source 1..1704: mol_type = other RNA
atgaaaacaattatcgctctcagttatattttctgccttcctttggggcaagacttgccgggcaatgataatagtacagcgactctctgtctcggacaccacgcagtaccgaacggcacacttgtgaagacaatcacagacgatcaaatcgaagtgacgaatgcaacagagcttgttcaatcatcatctaccggcaaaatctgcaataatccgcatagaatccttgatggaatcgactgcaccctgattgacgcactgttgggagatcctcattgcgatgtgtttcagaatgagacatgggacctttttgtcgagaggagcaaagcgttttccaactgctacccgtatgacgtgcctgattacgcgtcactccgctcacttgtagcatcaagcggtactctggagttcatcaccgaaggattcacctggacgggcgtaactcagaacggcggttctaacgcatgtaagagggggccagggtccggcttcttctcacggctcaactggttgactaagtctggttcaacatacccggtccttaacgttacaatgccgaataatgacaactttgataaactctacatctggggcattcaccatccatccacaaatcaagaacagacaagtttgtacgttcaggcgtcagggcgcgttaccgtgagtacaagaagatcacagcaaacaattatacccaatattgggtcccgaccctgggtaagaggactgtcctctcgcatctccatatactggaccattgtcaaaccgggcgacgtcctggttatcaatagtaatggaaaccttattgctccgcgcggctatttcaaaatgcgaactggaaagtcaagtataatgcgctcagacgcaccgatcgatacttgtatcagtgaatgcatcacccctaacgggtccataccgaacgataagcccttccagaatgtgaataaaatcacgtatggagcatgccccaaatacgtgaagcaaaacaccctcaagttggctacgggtatgcgcaacgtcccagaaaaacaaacgcgaggcttgtttggggcgatagcaggttttatcgagaacggctgggaaggaatgatcgatgggtggtacggctttcgccatcaaaactcagaaggaactgggcaggccgcagatcttaagtctacgcaagcggcgatagatcaaattaatggcaagttgaatagggtgatagagaagacgaacgagaagttccatcaaatagagaaagaattcagtgaagtagaggggcgaattcaggatttggaaaaatatgtcgaggatactaagatcgacctgtggagctataacgcagagcttctggtagcacttgagaaccagcatactattgatctcaccgattccgagatgaacaagctttttgagaagaccaggagacagttgcgcgagaatgccgaagaaatgggtaacggttgttttaagatttatcacaaatgtgacaacgcgtgtattgagtccattaggaatggtacatatgatcacgatgtgtatcgcgatgaagcacttaacaatcggttccaaataaagggcgtggagctcaagagtgggtataaagattggatcctctggatctcattcgcgatttcctgcttcctcctgtgcgttgtcttgctgggctttattatgtgggcatgtcagagaggtaacatccggtgcaacatatgtatctgataa

SEQ ID NO: 4 - RNA, 1993 nt, Human alphaherpesvirus 3
  source 1..1993: mol_type = other RNA
cgccaccatgttctatgaagctttgaaggccgaacttgtatacacccgagccgtgcatggtttccgcccgagagcgaactgcgtcgtactgtcagattacatccccagagtcgcgtgcaacatgggtacggttaataagcccgtggtgggggtgctgatgggttttgggataatcaccggaactctgcgcatcacaaatcctgtaagagcgtcagtgctgaggtatgacgatttccacatagacgaggacaagctggataccaattccgtttacgaaccatattatcactccgatcacgccgaatccagctgggtaaatcgaggagagtcctcacggaaggcatacgaccataactccccatatatatggcctcgcaatgattatgacggattcctggaaaacgcccatgagcaccacggcgtttataaccagggtcgcggtatcgattctggggaaaggttgatgcaaccaactcagatgtccgctcaggaagatttgggggatgacaccggaatccatgtgatacctacactcaatggtgatgatcgacacaaaattgtaaacgtggaccagaggcaatacggggacgtttttaagggagacctgaatccaaaaccacaaggacaacgactcatcgaggtttctgtagaagaaaaccatccgtttacgctccgcgcccccatacagcgcatatatggcgtgcgctatacggagacctggtcttttctccccagtctgacatgcactggtgatgcagctccagcaattcagcacatatgtctcaagcataccacctgtttccaagatgtagtagtggatgtggattgcgccgaaaacacgaaagaagaccagcttgccgagatttcataccggtttcaaggaaaaaaagaagccgaccagccgtggatagtagtgaatacgtccacgctcttcgatgaactggaattggaccctcctgaaattgaacccggagtcctgaaagttttgcggactgagaaacagtatctcggtgtgtacatctggaatatgaggggcagtgatggaacaagcacatacgcgacctttctggtgacatggaaaggtgacgaaaagactagaaacccaaccccagcagtgaccccccaacctagaggtgctgagttccacatgtggaattaccactcccatgtattctctgtcggagacaccttcagcttggcgatgcacctccaatacaagatccatgaagccccgttcgacctcttgttggagtggctctacgtaccaatagaccctacctgtcaaccaatgaggctttactctacatgcctttaccacccaaacgcaccacaatgtctttctcatatgaatagcggttgtacatttacatctccccatctcgcgcaaagagtcgcgagtacagtttaccaaaattgtgagcatgcggacaattacacagcatattgtcttgggatctcccacatggagccctcttttgggcttatacttcacgacgggggtacaacgcttaagttcgtcgatacgccagaatccctctctggcttgtatgtattcgtggtttattttaacggtcacgttgaggctgtggcctatactgttgttagcactgtagaccactttgttaacgccatagaagagagggggttcccgcccaccgctggccagccaccggccacaacgaagcctaaagagattacgccggtgaatcccggcacaagtccccttatccggtacgccgcatggacaggaggactggcggccgtcgtcctcctttgtctggttatattccttatatgcacggcaaagcgaatgcgggttaaggcttaccgagtggataaatctccatataatcagagcatgtattacgcagggctccctgtggacgactttgaggatagcgagtccacggatacagaagaggaatttggaaacgccatcggtgggagccatggagggtcatcttacaccgtttacattgacaaaactagatga

SEQ ID NO: 5 - RNA, 1072 nt, Human alphaherpesvirus 3
  source 1..1072: mol_type = other RNA
cgccaccatgttccttattcaatgcctcatatccgcagtcatcttctatattcaagttactaatgcacttatcttcaagggcgatcatgtcagcctgcaagtgaattcaagtcttacgtccattttgattccaatgcagaatgataactataccgaaattaagggacagctggtttttattggagaacaactgccgaccggtacaaactacagcgggaccctcgagctgctttacgcagacactgtggcattctgctttcggtcagtacaagttatcagatacgacggatgcccacgcattaggacatctgccttcatttcttgccgatacaagcatagttggcattacggaaactctaccgatagaatttcaactgaaccagatgccggtgtgatgctcaagataaccaaacctgggatcaacgacgcaggggtgtatgtcctcttggtgagattggaccattcaaggagtacggacgggtttatactgggcgtgaacgtctataccgcaggaagtcatcataacattcacggtgtcatttataccagccccagtctccagaatgggtacagcactcgagccctgttccagcaagcaagattgtgtgaccttccagccactcctaagggatcaggcacaagtctttttcaacatatgttggatctcagagcagggaaaagtcttgaggacaacccgtggctccatgaagacgtggttactactgaaacaaagtcagtggtcaaggagggaatcgagaaccatgtgtacccaactgacatgagcacgctgcctgaaaaatcactgaacgacccaccagagaatctgctgataataatacctattgtagcgagtgttatgattttgaccgcaatggtcatagttattgtaataagcgtgaaaaggagacgaatcaaaaaacatccgatatacaggccgaatacgaagacaagaagagggattcagaacgcgactccggagagtgatgtaatgctcgaagcagccatcgctcaacttgccaccattcgcgaagaaagccctccgcattccgtcgtaaatccttttgtcaagtga

SEQ ID NO: 6 - RNA, 2079 nt, Zika virus
  source 1..2079: mol_type = other RNA
atgcggagaggagcagacacatccgtgggaatcgtgggcctgctgctgaccacagcaatggcagccgaggtgaccaggagaggcagcgcctactatatgtacctggacagaaatgatgccggcgaggccatctcctttcccaccacactgggcatgaacaagtgctacatccagatcatggacctgggccacatgtgcgatgccaccatgagctatgagtgtccaatgctggacgagggcgtggagcccgacgatgtggattgctggtgtaataccacatctacatgggtggtgtacggcacctgtcaccacaagaagggagaggcacggcgcagcaggagagcagtgacactgccttcccactctaccaggaagctgcagacaagaagccagacctggctggagtccagggagtatacaaagcacctgatcagggtggagaactggatctttagaaatccaggattcgcactggctgccgccgccatcgcatggctgctgggcagctccaccagccagaaagtgatctacctggtcatgatcctgctgatcgcccctgcctattctatccggtgcatcggcgtgagcaatagggacttcgtggagggaatgtccggaggcacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcagatgtcccacccagggcgaggcctacctggacaagcagtccgatacacagtacgtgtgcaagcggaccctggtggacaggggatggggaaatggatgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactccccacgcgccgaggccaccctgggaggatttggctctctgggcctggactgcgagcctaggacaggcctggacttctccgatctgtactatctgaccatgaacaataagcactggctggtgcacaaggagtggtttcacgacatcccactgccatggcacgcaggagcagatacaggcaccccacactggaacaataaggaggccctggtggagttcaaggacgcccacgccaagcggcagacagtggtggtgctgggcagccaggagggagcagtgcacaccgccctggcaggcgccctggaggccgagatggacggagcaaagggccgcctgtctagcggccacctgaagtgcaggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggacccccctttcggcgatagctatatcgtgatcggcgtgggcgagaagaagatcacacaccactggcacagaagcggctccacaatcggcaaggcctttgaggcaaccgtgcggggagcaaagaggatggccgtgctgggcgacaccgcatgggatttcggatctgtgggaggcgccctgaacagcctgggcaagggcatccaccagatcttcggcgccgcctttaagtccctgttcggcggcatgagctggttttcccagatcctgatcggcacactgctgatgtggctgggcctgaacaccaagaatggctctatcagcctgatgtgcctggccctgggaggcgtgctgatcttcctgtccaccgccgtgtctgcc

SEQ ID NO: 7 - protein, 298 aa, Human orthopneumovirus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 8 - protein, 166 aa, Human orthopneumovirus
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNK

SEQ ID NO: 9 - protein, 574 aa, Human orthopneumovirus
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQSTNKAVVSLSNGVSVLTSKVLDLKNYINNQLLPIVNKQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 10 - protein, 566 aa, Influenza A virus
MKTIIALSYIFCLPLGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 11 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 12 - protein, 584 aa, Influenza B virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 13 - protein, 661 aa, Human alphaherpesvirus 3
MFYEALKAELVYTRAVHGFRPRANCVVLSDYIPRVACNMGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 14 - protein, 931 aa, Human alphaherpesvirus 3
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPTRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 15 - protein, 836 aa, Human alphaherpesvirus 3
MFALVLAVVILPLWTTANKSYVTPTPATRSIGHMSALLREYSDRNMSLKLEAFYPTGFDEELIKSLHWGNDRKHVFLVIVKVNPTTHEGDVGLVIFPTYLLSPYHFKAEHRAPFPAGRFGFLSHPVTPDVSFFDSSFAPYLTTQHLVAFTTFPPNPLVWHLERAETAATAERPFGVSLLPARPTVPKNTILEHKAHFATWDALARHTFFSAEAIITNSTLRIHVPLFGSVWPIRYWATGSVLLTSDSGRVEVNIGVGFMSSLISLSSGLPIELIVVPHTVKLNAVTSDTTWFQLNPPGPDPGPSYRVYLLGRGLDMNFSKHATVDICAYPEESLDYRYHLSMAHTEALRMTTKADQHDINEESYYHIAARIATSIMGRTTEYFLLDEIVDVQYQLKFLNYILMRIGAGAHPNTISGTSDLIFADPSQLHDELSLLFGQVKPANVDYFISYDEARDQLKTAYALSRGQDHVNALSLARRVIMSIYKGLLVKQNLNATERQALFFASMILLNFREGLENSSRVLDGRTTLLLMTSMCTAAHATQAALNIQEGLAYLNPSKHMFTIPNVYSPCMGSLRTDLTEEIHVMNLLSAIPTRPGLNEVLHTQLDESEIFDAAFKTMMIFTTWTAKDLHILHTHVPEVFTCQDAAARNGEYVLILPAVQGHSYVITRNKPQRGLVYSLADVDVYNPISVVYLSKDTCVSEHGVIETVALPHPDNLKECLYCGSVFLRYLTTGAIMDIIIIDSKDTERQLAAMGNSTIPPFNPDMHGDDSKAVLLFPNGTVVTLLGFERRQAIRMSGQYLGASLGGAFLAVVGFGIIGWMLCGNSRLREYNKIPLT

SEQ ID NO: 16 - protein, 160 aa, Human alphaherpesvirus 3
MASHKWLLQMIVFLKTITIAYCLHLQDDTPLFFGAKPLSDVSLIITEPCVSSVYEAWDYAAPPVSNLSEALSGIVVKTKCPVPEVILWFKDKQMAYWTNPYVTLKGLAQSVGEEHKSGDIRDALLDALSGVWVDSTPSSTNIPENGCVWGADRLFQRVCQ

SEQ ID NO: 17 - protein, 87 aa, Human alphaherpesvirus 3
MGSITASFILITMQILFFCEDSSGEPNFAERNFWHASCSARGVYIDGSMITTLFFYASLLGVCVALISLAYHACFRLFTRSVLRSTW

SEQ ID NO: 18 - protein, 354 aa, Human alphaherpesvirus 3
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 19 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIVIVMVTILLCCMTSCCSCLKGACSCGSCCKFDEDDSEPVLKGVKLHYT

SEQ ID NO: 20 - protein, 857 aa, Human gammaherpesvirus 4
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 21 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 22 - protein, 51 aa, Human papillomavirus
SQPLKQHYQIVTCCCGCDSNVRLVVQCTETDIREVQQLLLGTLNIVCPICA

SEQ ID NO: 23 - protein, 450 aa, Rabies lyssavirus
MDADKIVFKVNNQVVSLKPEIIVDQYEYKYPAIKDLKKPCITLGKAPDLNKAYKSVLSCMSAAKLDPDDVCSYLAAAMQFFEGTCPEDWTSYGILIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIIMNGGRLKRSHIRRYVSVSSNHQARPNSFAEFLNKTYSSDS

SEQ ID NO: 24 - protein, 11 aa, Rabies lyssavirus
MIDPGEVYDDP

SEQ ID NO: 25 - protein, 524 aa, Rabies lyssavirus
MVPQALLFVPLLVFPLCFGKFPIYTIPDKLGPWSPIDIHHLSCPNNLVVEDEGCTNLSGFSYMELKVGYISAIKTNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPMADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGL

SEQ ID NO: 26 - protein, 297 aa, Rabies lyssavirus
MSKIFVNPSAIRAGLADLEMAEETVDLINRNIEDNQAHLQGEPIEVDNLPEDMGRLHLDDGKSPNPGEMAKVGEGKYREDFQMDEGEDPSLLFQSYLDNVGVQIVRQIRSGERFFKIWSQTVEEIISYVAVNFPNPPGKSSEDKSTQTTGRELKKETTPTPSQRESQSSKARMAAQTASGPPALEWSATNEEDDLSVEAEIAHQIAESFSKKYKFPSRSSGILLYNFEQLKMNLDDIVKEAKNVPGVTRLARDGSKLPLRCVLGWVALANSKKFQLLVESNKLSKIMQDDLNRYTSC

SEQ ID NO: 27 - protein, 202 aa, Rabies lyssavirus
MNFLRKIVKNCRDEDTQKPSPVSAPLDDDDLWLPPPEYVPLKELTSKKNRRNFCINGGVKVCSPNGYSFGILRHILRSFDEIYSGNHRMIGLVKVVIGLALSGAPVPEGMNWVYKLRRTLIFQWADSRGPLEGEELEYSQEITWDDDTEFVGLQIRVSAKQCHIRGRIWCINMNSRAGQLWSDMSLQTQRSEEDKDSSLLLE

SEQ ID NO: 28 - protein, 904 aa, Human alphaherpesvirus 1
MRQGAPARGRRWFVVWALLGLTLGVLVASAAPSSPGTPGVAAATQAANGGPATPAPPAPGAPPTGDPKPKKNKKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 29 - protein, 698 aa, Human alphaherpesvirus 2
MHAIAPRLLLLFVLSGLPGTRGGSGVPGPINPPNNDVVFPGGSPVAQYCYAYPRLDDPGPLGSADAGRQDLPRRVVRHEPLGRSFLTGGLVLLAPPVRGFGAPNATYAARVTYYRLTRACRQPILLRQYGGCRGGEPPSPKTCGSYTYTYQGGGPPTRYALVNASLLVPIWDRAAETFEYQIELGGELHVGLLWVEVGGEGPGPTAPPQAARAEGGPCVPPVPAGRPWRSVPPVWYSAPNPGFRGLRFRERCLPPQTPAAPSDLPRVAFAPQSLLVGITGRTFIRMARPTEDVGVLPPHWAPGALDDGPYAPFPPRPRFRRALRTDPEGVDPDVRAPLTGRRLMALTEDASSDSPTSAPEKTPLPVSATAMAPSVDPSAEPTAPATTTPPDEMATQAATVAVTPEETAVASPPATASVESSPLPAAAATPGAGHTNTSSAPAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPDDDDSATGLAFRTPNPNKPPPARPGPIRPTLPPGILGPLAPNTPRPPAQAPAKDMPSGPTPQHIPLFWFLTASPALDILFIISTTIHTAAFVCLVALAAQLWRGRAGRRRYAHPSVRYVCLPPERD

SEQ ID NO: 30 - protein, 741 aa, Mycobacterium tuberculosis
MTDRVSVGNLRIARVLYDFVNNEALPGTDIDPDSFWAGVDKVVADLTPQNQALLNARDELQAQIDKWHRRRVIEPIDMDAYRQFLTEIGYLLPEPDDFTITTSGVDAEITTTAGPQLVVPVLNARFALNAANARWGSLYDALYGTDVIPETDGAEKGPTYNKVRGDKVIAYARKFLDDSVPLSSGSFGDATGFTVQDGQLVVALPDKSTGLANPGQFAGYTGAAESPTSVLLINHGLHIEILIDPESQVGTTDRAGVKDVILESAITTIMDFEDSVAAVDAADKVLGYRNWLGLNKGDLAAAVDKDGTAFLRVLNRDRNYTAPGGGQFTLPGRSLMFVRNVGHLMTNDAIVDTDGSEVFEGIMDALFTGLIAIHGLKASDVNGPLINSRTGSIYIVKPKMHGPAEVAFTCELFSRVEDVLGLPQNTMKIGIMDEERRTTVNLKACIKAAADRVVFINTGFLDRTGDEIHTSMEAGPMVRKGTMKSQPWILAYEDHNVDAGLAAGFSGRAQVGKGMWTMTELMADMVETKIAQPRAGASTAWVPSPTAATLHALHYHQVDVAAVQQGLAGKRRATIEQLLTIPLAKELAWAPDEIREEVDNNCQSILGYVVRWVDQGVGCSKVPDIHDVALMEDRATLRISSQLLANWLRHGVITSADVRASLERMAPLVDRQNAGDVAYRPMAPNFDDSIAFLAAQELILSGAQQPNGYTEPILHRRRREFKARAAEKPAPSDRAGDDAAR

SEQ ID NO: 31 - protein, 299 aa, Mycobacterium tuberculosis
MKGRSALLRALWIAALSFGLGGVAVAAEPTAKAAPYENLMVPSPSMGRDIPVAFLAGGPHAVYLLDAFNAGPDVSNWVTAGNAMNTLAGKGISVVAPAGGAYSMYTNWEQDGSKQWDTFLSAELPDWLAANRGLAPGGHAAVGAAQGGYGAMALAAFHPDRFGFAGSMSGFLYPSNTTTNGAIAAGMQQFGGVDTNGMWGAPQLGRWKWHDPWVHASLLAQNNTRVWVWSPTNPGASDPAAMIGQTAEAMGNSRMFYNQYRSVGGHNGHFDFPASGDNGWGSWAPQLGAMSGDIVGAIR

SEQ ID NO: 32 - protein, 3181 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRAPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPTSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTWKRSGALWDVPASKEVKKGETTDGVYRVMTRRLLGSTQVGVGIMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTILEDSVPAEVWTRHGEKRVLKPRWMDARVCSDHAALKSFKEFAAGKRGAAFGVMEALGTLPGHMTERFQEAIDNLAVLMRAEIGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSCIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMA

SEQ ID NO: 33 - protein, 397 aa, Chlamydia trachomatis
MKKLLKSVLVFAALSSASSLQALPVGNPAEPSLMIDGILWEGFGGDPCDPCTTWCDAISMRVGYYGDFVFDRVLKTDVNKEFQMGAAPTTSDVAGLQNDPTINVARPNPAYGKHMQDAEMFTNAAYMALNIWDRFDVFCTLGATTGYLKGNSASFNLVGLFGTKTQSSSFNTAKLIPNTALNEAVVELYINTTFAWSVGARAALWECGCATLGASFQYAQSKPKVEELNVLCNASEFTINKPKGYVGAEFPLNITAGTEAATGTKDASIDYHEWQASLALSYRLNMFTPYIGVKWSRVSFDADTIRIAQPKLAEAILDVTTLNRTTAGKGSVVSAGTDNELADTMQIVSLQLNKMKSRKSCGIAVGTTIVDADKYAVTVEARLIDERAAHVNAQFRF

SEQ ID NO: 34 - RNA, 7561 nt, Venezuelan equine encephalitis virus
  source 1..7561: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 35 - protein, 51 aa, Venezuelan equine encephalitis virus
RELPVLDSAAFNVECFKKYACNNEYWETFKENPIRLTEENVVNYITKLKGP

SEQ ID NO: 36 - protein, 41 aa, Venezuelan equine encephalitis virus
TQMRELPVLDSAAFNVECFKKYACNNEYWETFKENPIRLTE

SEQ ID NO: 37 - protein, 536 aa, Venezuelan equine encephalitis virus
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 38 - RNA, 9847 nt, synthetic construct
  misc_feature 1..9847: note = Description of Artificial Sequence: Synthetic polynucleotide
  source 1..9847: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgttctatgaagctttgaaggccgaacttgtatacacccgagccgtgcatggtttccgcccgagagcgaactgcgtcgtactgtcagattacatccccagagtcgcgtgcaacatgggtacggttaataagcccgtggtgggggtgctgatgggttttgggataatcaccggaactctgcgcatcacaaatcctgtaagagcgtcagtgctgaggtatgacgatttccacatagacgaggacaagctggataccaattccgtttacgaaccatattatcactccgatcacgccgaatccagctgggtaaatcgaggagagtcctcacggaaggcatacgaccataactccccatatatatggcctcgcaatgattatgacggattcctggaaaacgcccatgagcaccacggcgtttataaccagggtcgcggtatcgattctggggaaaggttgatgcaaccaactcagatgtccgctcaggaagatttgggggatgacaccggaatccatgtgatacctacactcaatggtgatgatcgacacaaaattgtaaacgtggaccagaggcaatacggggacgtttttaagggagacctgaatccaaaaccacaaggacaacgactcatcgaggtttctgtagaagaaaaccatccgtttacgctccgcgcccccatacagcgcatatatggcgtgcgctatacggagacctggtcttttctccccagtctgacatgcactggtgatgcagctccagcaattcagcacatatgtctcaagcataccacctgtttccaagatgtagtagtggatgtggattgcgccgaaaacacgaaagaagaccagcttgccgagatttcataccggtttcaaggaaaaaaagaagccgaccagccgtggatagtagtgaatacgtccacgctcttcgatgaactggaattggaccctcctgaaattgaacccggagtcctgaaagttttgcggactgagaaacagtatctcggtgtgtacatctggaatatgaggggcagtgatggaacaagcacatacgcgacctttctggtgacatggaaaggtgacgaaaagactagaaacccaaccccagcagtgaccccccaacctagaggtgctgagttccacatgtggaattaccactcccatgtattctctgtcggagacaccttcagcttggcgatgcacctccaatacaagatccatgaagccccgttcgacctcttgttggagtggctctacgtaccaatagaccctacctgtcaaccaatgaggctttactctacatgcctttaccacccaaacgcaccacaatgtctttctcatatgaatagcggttgtacatttacatctccccatctcgcgcaaagagtcgcgagtacagtttaccaaaattgtgagcatgcggacaattacacagcatattgtcttgggatctcccacatggagccctcttttgggcttatacttcacgacgggggtacaacgcttaagttcgtcgatacgccagaatccctctctggcttgtatgtattcgtggtttattttaacggtcacgttgaggctgtggcctatactgttgttagcactgtagaccactttgttaacgccatagaagagagggggttcccgcccaccgctggccagccaccggccacaacgaagcctaaagagattacgccggtgaatcccggcacaagtccccttatccggtacgccgcatggacaggaggactggcggccgtcgtcctcctttgtctggttatattccttatatgcacggcaaagcgaatgcgggttaaggcttaccgagtggataaatctccatataatcagagcatgtattacgcagggctccctgtggacgactttgaggatagcgagtccacggatacagaagaggaatttggaaacgccatcggtgggagccatggagggtcatcttacaccgtttacattgacaaaactagatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 39 - RNA, 8926 nt, synthetic construct
  misc_feature 1..8926: note = Description of Artificial Sequence: Synthetic polynucleotide
  source 1..8926: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgttccttattcaatgcctcatatccgcagtcatcttctatattcaagttactaatgcacttatcttcaagggcgatcatgtcagcctgcaagtgaattcaagtcttacgtccattttgattccaatgcagaatgataactataccgaaattaagggacagctggtttttattggagaacaactgccgaccggtacaaactacagcgggaccctcgagctgctttacgcagacactgtggcattctgctttcggtcagtacaagttatcagatacgacggatgcccacgcattaggacatctgccttcatttcttgccgatacaagcatagttggcattacggaaactctaccgatagaatttcaactgaaccagatgccggtgtgatgctcaagataaccaaacctgggatcaacgacgcaggggtgtatgtcctcttggtgagattggaccattcaaggagtacggacgggtttatactgggcgtgaacgtctataccgcaggaagtcatcataacattcacggtgtcatttataccagccccagtctccagaatgggtacagcactcgagccctgttccagcaagcaagattgtgtgaccttccagccactcctaagggatcaggcacaagtctttttcaacatatgttggatctcagagcagggaaaagtcttgaggacaacccgtggctccatgaagacgtggttactactgaaacaaagtcagtggtcaaggagggaatcgagaaccatgtgtacccaactgacatgagcacgctgcctgaaaaatcactgaacgacccaccagagaatctgctgataataatacctattgtagcgagtgttatgattttgaccgcaatggtcatagttattgtaataagcgtgaaaaggagacgaatcaaaaaacatccgatatacaggccgaatacgaagacaagaagagggattcagaacgcgactccggagagtgatgtaatgctcgaagcagccatcgctcaacttgccaccattcgcgaagaaagccctccgcattccgtcgtaaatccttttgtcaagtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 40 - RNA, 9733 nt, synthetic construct
  misc_feature 1..9733: note = Description of Artificial Sequence: Synthetic polynucleotide
  source 1..9733: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgggtacggttaataagcccgtggtgggggtgctgatgggttttgggataatcaccggaactctgcgcatcacaaatcctgtaagagcgtcagtgctgaggtatgacgatttccacatagacgaggacaagctggataccaattccgtttacgaaccatattatcactccgatcacgccgaatccagctgggtaaatcgaggagagtcctcacggaaggcatacgaccataactccccatatatatggcctcgcaatgattatgacggattcctggaaaacgcccatgagcaccacggcgtttataaccagggtcgcggtatcgattctggggaaaggttgatgcaaccaactcagatgtccgctcaggaagatttgggggatgacaccggaatccatgtgatacctacactcaatggtgatgatcgacacaaaattgtaaacgtggaccagaggcaatacggggacgtttttaagggagacctgaatccaaaaccacaaggacaacgactcatcgaggtttctgtagaagaaaaccatccgtttacgctccgcgcccccatacagcgcatatatggcgtgcgctatacggagacctggtcttttctccccagtctgacatgcactggtgatgcagctccagcaattcagcacatatgtctcaagcataccacctgtttccaagatgtagtagtggatgtggattgcgccgaaaacacgaaagaagaccagcttgccgagatttcataccggtttcaaggaaaaaaagaagccgaccagccgtggatagtagtgaatacgtccacgctcttcgatgaactggaattggaccctcctgaaattgaacccggagtcctgaaagttttgcggactgagaaacagtatctcggtgtgtacatctggaatatgaggggcagtgatggaacaagcacatacgcgacctttctggtgacatggaaaggtgacgaaaagactagaaacccaaccccagcagtgaccccccaacctagaggtgctgagttccacatgtggaattaccactcccatgtattctctgtcggagacaccttcagcttggcgatgcacctccaatacaagatccatgaagccccgttcgacctcttgttggagtggctctacgtaccaatagaccctacctgtcaaccaatgaggctttactctacatgcctttaccacccaaacgcaccacaatgtctttctcatatgaatagcggttgtacatttacatctccccatctcgcgcaaagagtcgcgagtacagtttaccaaaattgtgagcatgcggacaattacacagcatattgtcttgggatctcccacatggagccctcttttgggcttatacttcacgacgggggtacaacgcttaagttcgtcgatacgccagaatccctctctggcttgtatgtattcgtggtttattttaacggtcacgttgaggctgtggcctatactgttgttagcactgtagaccactttgttaacgccatagaagagagggggttcccgcccaccgctggccagccaccggccacaacgaagcctaaagagattacgccggtgaatcccggcacaagtccccttatccggtacgccgcatggacaggaggactggcggccgtcgtcctcctttgtctggttatattccttatatgcacggcaaagcgaatgcgggttaaggcttaccgagtggataaatctccatataatcagagcatgtattacgcagggctccctgtggacgactttgaggatagcgagtccacggatacagaagaggaatttggaaacgccatcggtgggagccatggagggtcatcttacaccgtttacattgacaaaactagatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 41 - RNA, 10984 nt, synthetic construct
  misc_feature 1..10984: note = Description of Artificial Sequence: Synthetic polynucleotide
  source 1..10984: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgttccttattcaatgcctcatatccgcagtcatcttctatattcaagttactaatgcacttatcttcaagggcgatcatgtcagcctgcaagtgaattcaagtcttacgtccattttgattccaatgcagaatgataactataccgaaattaagggacagctggtttttattggagaacaactgccgaccggtacaaactacagcgggaccctcgagctgctttacgcagacactgtggcattctgctttcggtcagtacaagttatcagatacgacggatgcccacgcattaggacatctgccttcatttcttgccgatacaagcatagttggcattacggaaactctaccgatagaatttcaactgaaccagatgccggtgtgatgctcaagataaccaaacctgggatcaacgacgcaggggtgtatgtcctcttggtgagattggaccattcaaggagtacggacgggtttatactgggcgtgaacgtctataccgcaggaagtcatcataacattcacggtgtcatttataccagccccagtctccagaatgggtacagcactcgagccctgttccagcaagcaagattgtgtgaccttccagccactcctaagggatcaggcacaagtctttttcaacatatgttggatctcagagcagggaaaagtcttgaggacaacccgtggctccatgaagacgtggttactactgaaacaaagtcagtggtcaaggagggaatcgagaaccatgtgtacccaactgacatgagcacgctgcctgaaaaatcactgaacgacccaccagagaatctgctgataataatacctattgtagcgagtgttatgattttgaccgcaatggtcatagttattgtaataagcgtgaaaaggagacgaatcaaaaaacatccgatatacaggccgaatacgaagacaagaagagggattcagaacgcgactccggagagtgatgtaatgctcgaagcagccatcgctcaacttgccaccattcgcgaagaaagccctccgcattccgtcgtaaatccttttgtcaagcggaggaaaaggggatccggggaaggccggggtagtttgctgacgtgcggagatgttgaagagaacccagggccgatgttctatgaagctttgaaggccgaacttgtatacacccgagccgtgcatggtttccgcccgagagcgaactgcgtcgtactgtcagattacatccccagagtcgcgtgcaacatgggtacggttaataagcccgtggtgggggtgctgatgggttttgggataatcaccggaactctgcgcatcacaaatcctgtaagagcgtcagtgctgaggtatgacgatttccacatagacgaggacaagctggataccaattccgtttacgaaccatattatcactccgatcacgccgaatccagctgggtaaatcgaggagagtcctcacggaaggcatacgaccataactccccatatatatggcctcgcaatgattatgacggattcctggaaaacgcccatgagcaccacggcgtttataaccagggtcgcggtatcgattctggggaaaggttgatgcaaccaactcagatgtccgctcaggaagatttgggggatgacaccggaatccatgtgatacctacactcaatggtgatgatcgacacaaaattgtaaacgtggaccagaggcaatacggggacgtttttaagggagacctgaatccaaaaccacaaggacaacgactcatcgaggtttctgtagaagaaaaccatccgtttacgctccgcgcccccatacagcgcatatatggcgtgcgctatacggagacctggtcttttctccccagtctgacatgcactggtgatgcagctccagcaattcagcacatatgtctcaagcataccacctgtttccaagatgtagtagtggatgtggattgcgccgaaaacacgaaagaagaccagcttgccgagatttcataccggtttcaaggaaaaaaagaagccgaccagccgtggatagtagtgaatacgtccacgctcttcgatgaactggaattggaccctcctgaaattgaacccggagtcctgaaagttttgcggactgagaaacagtatctcggtgtgtacatctggaatatgaggggcagtgatggaacaagcacatacgcgacctttctggtgacatggaaaggtgacgaaaagactagaaacccaaccccagcagtgaccccccaacctagaggtgctgagttccacatgtggaattaccactcccatgtattctctgtcggagacaccttcagcttggcgatgcacctccaatacaagatccatgaagccccgttcgacctcttgttggagtggctctacgtaccaatagaccctacctgtcaaccaatgaggctttactctacatgcctttaccacccaaacgcaccacaatgtctttctcatatgaatagcggttgtacatttacatctccccatctcgcgcaaagagtcgcgagtacagtttaccaaaattgtgagcatgcggacaattacacagcatattgtcttgggatctcccacatggagccctcttttgggcttatacttcacgacgggggtacaacgcttaagttcgtcgatacgccagaatccctctctggcttgtatgtattcgtggtttattttaacggtcacgttgaggctgtggcctatactgttgttagcactgtagaccactttgttaacgccatagaagagagggggttcccgcccaccgctggccagccaccggccacaacgaagcctaaagagattacgccggtgaatcccggcacaagtccccttatccggtacgccgcatggacaggaggactggcggccgtcgtcctcctttgtctggttatattccttatatgcacggcaaagcgaatgcgggttaaggcttaccgagtggataaatctccatataatcagagcatgtattacgcagggctccctgtggacgactttgaggatagcgagtccacggatacagaagaggaatttggaaacgccatcggtgggagccatggagggtcatcttacaccgtttacattgacaaaactagatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 42 - RNA, 11498 nt, synthetic construct
  misc_feature 1..11498: note = Description of Artificial Sequence: Synthetic polynucleotide
  source 1..11498: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgttccttattcaatgcctcatatccgcagtcatcttctatattcaagttactaatgcacttatcttcaagggcgatcatgtcagcctgcaagtgaattcaagtcttacgtccattttgattccaatgcagaatgataactataccgaaattaagggacagctggtttttattggagaacaactgccgaccggtacaaactacagcgggaccctcgagctgctttacgcagacactgtggcattctgctttcggtcagtacaagttatcagatacgacggatgcccacgcattaggacatctgccttcatttcttgccgatacaagcatagttggcattacggaaactctaccgatagaatttcaactgaaccagatgccggtgtgatgctcaagataaccaaacctgggatcaacgacgcaggggtgtatgtcctcttggtgagattggaccattcaaggagtacggacgggtttatactgggcgtgaacgtctataccgcaggaagtcatcataacattcacggtgtcatttataccagccccagtctccagaatgggtacagcactcgagccctgttccagcaagcaagattgtgtgaccttccagccactcctaagggatcaggcacaagtctttttcaacatatgttggatctcagagcagggaaaagtcttgaggacaacccgtggctccatgaagacgtggttactactgaaacaaagtcagtggtcaaggagggaatcgagaaccatgtgtacccaactgacatgagcacgctgcctgaaaaatcactgaacgacccaccagagaatctgctgataataatacctattgtagcgagtgttatgattttgaccgcaatggtcatagttattgtaataagcgtgaaaaggagacgaatcaaaaaacatccgatatacaggccgaatacgaagacaagaagagggattcagaacgcgactccggagagtgatgtaatgctcgaagcagccatcgctcaacttgccaccattcgcgaagaaagccctccgcattccgtcgtaaatccttttgtcaagtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgttctatgaagctttgaaggccgaacttgtatacacccgagccgtgcatggtttccgcccgagagcgaactgcgtcgtactgtcagattacatccccagagtcgcgtgcaacatgggtacggttaataagcccgtggtgggggtgctgatgggttttgggataatcaccggaactctgcgcatcacaaatcctgtaagagcgtcagtgctgaggtatgacgatttccacatagacgaggacaagctggataccaattccgtttacgaaccatattatcactccgatcacgccgaatccagctgggtaaatcgaggagagtcctcacggaaggcatacgaccataactccccatatatatggcctcgcaatgattatgacggattcctggaaaacgcccatgagcaccacggcgtttataaccagggtcgcggtatcgattctggggaaaggttgatgcaaccaactcagatgtccgctcaggaagatttgggggatgacaccggaatccatgtgatacctacactcaatggtgatgatcgacacaaaattgtaaacgtggaccagaggcaatacggggacgtttttaagggagacctgaatccaaaaccacaaggacaacgactcatcgaggtttctgtagaagaaaaccatccgtttacgctccgcgcccccatacagcgcatatatggcgtgcgctatacggagacctggtcttttctccccagtctgacatgcactggtgatgcagctccagcaattcagcacatatgtctcaagcataccacctgtttccaagatgtagtagtggatgtggattgcgccgaaaacacgaaagaagaccagcttgccgagatttcataccggtttcaaggaaaaaaagaagccgaccagccgtggatagtagtgaatacgtccacgctcttcgatgaactggaattggaccctcctgaaattgaacccggagtcctgaaagttttgcggactgagaaacagtatctcggtgtgtacatctggaatatgaggggcagtgatggaacaagcacatacgcgacctttctggtgacatggaaaggtgacgaaaagactagaaacccaaccccagcagtgaccccccaacctagaggtgctgagttccacatgtggaattaccactcccatgtattctctgtcggagacaccttcagcttggcgatgcacctccaatacaagatccatgaagccccgttcgacctcttgttggagtggctctacgtaccaatagaccctacctgtcaaccaatgaggctttactctacatgcctttaccacccaaacgcaccacaatgtctttctcatatgaatagcggttgtacatttacatctccccatctcgcgcaaagagtcgcgagtacagtttaccaaaattgtgagcatgcggacaattacacagcatattgtcttgggatctcccacatggagccctcttttgggcttatacttcacgacgggggtacaacgcttaagttcgtcgatacgccagaatccctctctggcttgtatgtattcgtggtttattttaacggtcacgttgaggctgtggcctatactgttgttagcactgtagaccactttgttaacgccatagaagagagggggttcccgcccaccgctggccagccaccggccacaacgaagcctaaagagattacgccggtgaatcccggcacaagtccccttatccggtacgccgcatggacaggaggactggcggccgtcgtcctcctttgtctggttatattccttatatgcacggcaaagcgaatgcgggttaaggcttaccgagtggataaatctccatataatcagagcatgtattacgcagggctccctgtggacgactttgaggatagcgagtccacggatacagaagaggaatttggaaacgccatcggtgggagccatggagggtcatcttacaccgtttacattgacaaaactagatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 43 - RNA, 9373 nt, synthetic construct
  misc_feature 1..9373: note = Description of Artificial Sequence: Synthetic polynucleotide
  source 1..9373: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtaccgtaaataagcccgttgtcggtgtcttgatgggctttggaatcattactggaactttgaggattacgaaccctgtcagagcctctgttcttcggtacgacgacttccacattgacgaagacaagttggataccaactccgtatacgagccgtattaccactcagatcatgccgaatcatcatgggtcaatcgaggcgaatcttcccgaaaggcgtacgatcataacagtccctacatatggcccagaaacgattacgacggctttctcgagaacgcgcatgagcatcatggggtgtataatcaagggagaggcatagattctggtgaacgcttgatgcaaccaactcagatgtccgcacaagaagatcttggggacgacacaggaatacacgtcatccctacactgaacggcgatgatcggcataagatcgtaaatgtggatcaacggcaatatggcgacgtgtttaagggggaccttaatcccaagccccagggacaacggttgattgaggtatctgttgaggaaaaccacccctttacgctgcgggcgccgattcagcgcatctacggtgttcgctatacggagacgtggagtttcctgccatctctcacatgtaccggggacgctgcaccggcaattcaacatatttgcctgaaacacacgacatgctttcaagacgttgtagtggatgtggactgtgcggagaataccaaagaggaccaacttgcggaaataagctaccgattccaagggaagaaggaggccgaccagccatggattgttgttaatacatctacactttttgatgaactcgaacttgacccacccgagattgaacctggcgttttgaaagtccttagaacagaaaaacaataccttggtgtatatatatggaacatgagggggtccgacggcacgtctacctatgccacctttctggtcacatggaaaggagatgaaaagactcgcaatcctaccccggcggtcactcctcaacccagaggcgctgagttccatatgtggaattatcattcacatgtcttctccgttggcgacacttttagcttggcgatgcatctgcagtacaaaatacatgaggcgccgttcgaccttctccttgaatggctctatgtgcccattgatcctacttgtcaaccgatgaggctttactctacgtgcctctaccacccgaatgcgcctcaatgcctgtcccatatgaattccgggtgtacctttacatcccctcatcttgctcagcgagttgctagcacggtttaccagaactgcgagcatgcggacaattatacagcctactgtcttggcataagtcacatggaacctagcttcggcctgattctccatgatggaggaacaacgcttaaattcgtagacacccccgaatcattgagcggtttgtatgtttttgtagtttatttcaatgggcatgtggaggcggtagcttacaccgtcgtgtcaacagttgaccattttgtcaacgccatcgaagaaagaggatttccgccgaccgcgggtcagccgcccgctactaccaaacctaaggaaattactcccgtgaaccccggcaccagtccgctgataagatatgcggcttggacgggaggcttggcgtgaggcgcgccatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 44 - RNA, 9937 nt, synthetic construct
  misc_feature 1..9937: note = Description of Artificial Sequence: Synthetic polynucleotide
  source 1..9937: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgcggagaggagcagacacatccgtgggaatcgtgggcctgctgctgaccacagcaatggcagccgaggtgaccaggagaggcagcgcctactatatgtacctggacagaaatgatgccggcgaggccatctcctttcccaccacactgggcatgaacaagtgctacatccagatcatggacctgggccacatgtgcgatgccaccatgagctatgagtgtccaatgctggacgagggcgtggagcccgacgatgtggattgctggtgtaataccacatctacatgggtggtgtacggcacctgtcaccacaagaagggagaggcacggcgcagcaggagagcagtgacactgccttcccactctaccaggaagctgcagacaagaagccagacctggctggagtccagggagtatacaaagcacctgatcagggtggagaactggatctttagaaatccaggattcgcactggctgccgccgccatcgcatggctgctgggcagctccaccagccagaaagtgatctacctggtcatgatcctgctgatcgcccctgcctattctatccggtgcatcggcgtgagcaatagggacttcgtggagggaatgtccggaggcacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcagatgtcccacccagggcgaggcctacctggacaagcagtccgatacacagtacgtgtgcaagcggaccctggtggacaggggatggggaaatggatgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactccccacgcgccgaggccaccctgggaggatttggctctctgggcctggactgcgagcctaggacaggcctggacttctccgatctgtactatctgaccatgaacaataagcactggctggtgcacaaggagtggtttcacgacatcccactgccatggcacgcaggagcagatacaggcaccccacactggaacaataaggaggccctggtggagttcaaggacgcccacgccaagcggcagacagtggtggtgctgggcagccaggagggagcagtgcacaccgccctggcaggcgccctggaggccgagatggacggagcaaagggccgcctgtctagcggccacctgaagtgcaggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggacccccctttcggcgatagctatatcgtgatcggcgtgggcgagaagaagatcacacaccactggcacagaagcggctccacaatcggcaaggcctttgaggcaaccgtgcggggagcaaagaggatggccgtgctgggcgacaccgcatgggatttcggatctgtgggaggcgccctgaacagcctgggcaagggcatccaccagatcttcggcgccgcctttaagtccctgttcggcggcatgagctggttttcccagatcctgatcggcacactgctgatgtggctgggcctgaacaccaagaatggctctatcagcctgatgtgcctggccctgggaggcgtgctgatcttcctgtccaccgccgtgtctgcctgaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 45 - RNA, 9457 nt, synthetic construct
  misc_feature 1..9457: note = Description of Artificial Sequence: Synthetic polynucleotide
  source 1..9457: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagctgctcatccttaaagcgaatgctataactactatacttacagctgtaaccttttgcttcgcctctggccaaaacattactgaggagttttatcaatccacttgcagcgcagtctcaaaaggatatctgtcagcattgcgcaccgggtggtacaccagcgttatcactatcgagctttccaacattaaagagaataagtgtaacggcacagacgcgaaagtcaaactcattaagcaagaacttgataaatacaaaaatgcagttactgaacttcagttgctcatgcaatcaaccccagcaacgaacaatagggccaggagagaattgccgaggtttatgaactacacacttaataacgctaaaaagactaacgttacgctctctaagaagcggaagcgcaggtttctcgggttccttctgggggttggcagtgcaatagcatccggcgtcgcggtatcaaaggtgcttcatcttgaaggagaagttaacaaaatcaaaagcgccttgctttcaactaataaggcagtagtatcattgtctaacggtgtcagcgtcttgacttccaaggttttggatttgaaaaactatatcgacaaacaacttctcccgatcgttaacaagcagtcatgcagtatctccaacatcgaaaccgtcatcgaattccaacagaaaaacaatagactgcttgaaattactagagagttttcagtgaacgctggcgttaccactccggtatccacttatatgctcactaatagcgaactgctgtctctgataaatgacatgccaataactaacgaccagaagaaacttatgagtaacaacgtccagatcgtgagacagcaatcatatagcattatgagcataattaaggaggaggtccttgcatacgtagtccagctcccactgtacggggttatcgacacgccatgttggaagcttcatacttcccccttgtgcaccacgaacacgaaggaagggtctaacatttgtctcacgcgcactgatcgggggtggtactgtgacaacgccgggtcagtgtcatttttccctcaggccgagacctgcaaggtccaatcaaaccgggtattctgtgatactatgaactccctgactctgccttctgaagttaacctgtgtaatgtagatatattcaatcctaaatacgactgcaagataatgaccagcaaaaccgacgtgtcctcatctgtcatcacttcccttggtgctatagtaagctgctatggcaaaacgaaatgcaccgcgagtaataaaaatcgcggtatcatcaagacatttagtaacggctgcgattatgtttccaacaaaggtgttgacaccgtatctgtggggaataccctgtattacgtaaataagcaggaagggaaatctctctacgtgaagggggaaccgataatcaacttttatgacccgctggtttttccgtccgacgaatttgacgcgagtatctcccaagtcaacgagaaaattaaccaaagcctcgcgttcataagaaaatccgatgagctgctgcataatgtaaacgcgggcaaatcaactaccaacatcatgattacaacaataatcatcgtgataatcgtgatcctgctttcacttatcgctgtcgggcttcttctctattgtaaggcccggagtactcccgtgaccttgtctaaggatcagctctcaggtatcaacaatattgcattcagcaattgaggcgcgccatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 46 - RNA, 8755 nt, synthetic construct
  misc_feature 1..8755: note = Description of Artificial Sequence: Synthetic polynucleotide
  source 1..8755: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtccaaaaataaagaccagcgaacggccaagactctggagaggacttgggataccctgaatcatctgttgtttatttcaagttgcctttataaattgaatctcaaatccgtcgcgcaaatcacgctgagcatactcgcaatgattatctcaacttccttgataattgccgccataatcttcattgcaagtgcaaatcataaagtgactcctacaaccgcaataatacaggacgcgaccagccaaattaagaacacgactcccacgtatctcacccaaaatccccaactcggaattagcccaagtaatccgtcagagattacttcacaaatcaccaccatactggcgagcacaactccaggcgtaaaatccaccctccaatctactaccgtcaagactaaaaataccactactactcagacacaacctagtaaacctacgactaagcagcgccagaataaacccccaagcaaaccaaacaacgacttccattttgaagttttcaacttcgtgccttgttctatctgctctaataatccaacatgctgggccatctgcaagcgcattccgaataagaaacccggtaagaaaactacaacgaaacctaccaagaagcctaccctgaaaacgacaaagaaagacccgaaaccgcaaacaaccaaaagtaaagaagtgcccacaactaaacccacagaagaaccaacgataaacacgaccaaaaccaacataataactacgctgcttaccagcaacacgacaggcaacccggaattgaccagtcagatggaaacttttcattcaaccagcagcgaaggtaatccaagtcctagtcaggtgtctacgacatctgaatatccatctcagcctagttcccctccgaacacgccgcgccagtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 47 - RNA, 9559 nt, synthetic construct
  misc_feature 1..9559: note = Description of Artificial Sequence: Synthetic polynucleotide
  source 1..9559: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaaaacaattatcgctctcagttatattttctgccttcctttggggcaagacttgccgggcaatgataatagtacagcgactctctgtctcggacaccacgcagtaccgaacggcacacttgtgaagacaatcacagacgatcaaatcgaagtgacgaatgcaacagagcttgttcaatcatcatctaccggcaaaatctgcaataatccgcatagaatccttgatggaatcgactgcaccctgattgacgcactgttgggagatcctcattgcgatgtgtttcagaatgagacatgggacctttttgtcgagaggagcaaagcgttttccaactgctacccgtatgacgtgcctgattacgcgtcactccgctcacttgtagcatcaagcggtactctggagttcatcaccgaaggattcacctggacgggcgtaactcagaacggcggttctaacgcatgtaagagggggccagggtccggcttcttctcacggctcaactggttgactaagtctggttcaacatacccggtccttaacgttacaatgccgaataatgacaactttgataaactctacatctggggcattcaccatccatccacaaatcaagaacagacaagtttgtacgttcaggcgtcagggcgcgttaccgtgagtacaagaagatcacagcaaacaattatacccaatattgggtcccgaccctgggtaagaggactgtcctctcgcatctccatatactggaccattgtcaaaccgggcgacgtcctggttatcaatagtaatggaaaccttattgctccgcgcggctatttcaaaatgcgaactggaaagtcaagtataatgcgctcagacgcaccgatcgatacttgtatcagtgaatgcatcacccctaacgggtccataccgaacgataagcccttccagaatgtgaataaaatcacgtatggagcatgccccaaatacgtgaagcaaaacaccctcaagttggctacgggtatgcgcaacgtcccagaaaaacaaacgcgaggcttgtttggggcgatagcaggttttatcgagaacggctgggaaggaatgatcgatgggtggtacggctttcgccatcaaaactcagaaggaactgggcaggccgcagatcttaagtctacgcaagcggcgatagatcaaattaatggcaagttgaatagggtgatagagaagacgaacgagaagttccatcaaatagagaaagaattcagtgaagtagaggggcgaattcaggatttggaaaaatatgtcgaggatactaagatcgacctgtggagctataacgcagagcttctggtagcacttgagaaccagcatactattgatctcaccgattccgagatgaacaagctttttgagaagaccaggagacagttgcgcgagaatgccgaagaaatgggtaacggttgttttaagatttatcacaaatgtgacaacgcgtgtattgagtccattaggaatggtacatatgatcacgatgtgtatcgcgatgaagcacttaacaatcggttccaaataaagggcgtggagctcaagagtgggtataaagattggatcctctggatctcattcgcgatttcctgcttcctcctgtgcgttgtcttgctgggctttattatgtgggcatgtcagagaggtaacatccggtgcaacatatgtatctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 48 - RNA, 7862 nt, synthetic construct
  misc_feature 1..7862: note = Description of Artificial Sequence: Synthetic polynucleotide
  misc_difference 7562
  source 1..7862: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 49 - RNA, 7862 nt, synthetic construct; identical to SEQ ID NO: 48
  source 1..7862: mol_type = other RNA

SEQ ID NO: 50 - RNA, 7862 nt, synthetic construct
  source 1..7862: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 51 - RNA, 7862 nt, synthetic construct; identical to SEQ ID NO: 48
  source 1..7862: mol_type = other RNA

SEQ ID NO: 52 - RNA, 7862 nt, synthetic construct; identical to SEQ ID NO: 48
  source 1..7862: mol_type = other RNA

SEQ ID NO: 53 - RNA, 11701 nt, synthetic construct
  misc_feature 1..11701: note = Description of Artificial Sequence: Synthetic polynucleotide
  source 1..11701: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 54 - RNA, 7862 nt, synthetic construct
  source 1..7862: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccntaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa